(19) United States
(12) Patent Application Publication
(10) Pub. No.: US 2026/0328769 A1
(43) Pub. Date: Oct. 22, 2026

(54) GRANZYME B TARGETED IMAGING PROBES, KITS, METHOD OF MAKING, AND METHODS OF USE THEREOF

(71) Applicant: University of Virginia Patent Foundation, Charlottesville, VA (US)

(72) Inventors: Jinghang Xie, Glen Allen, VA (US); Huy Hoang Pham, Charlottesville, VA (US); Jinhui Zhu, Charlottesville, VA (US)

(21) Appl. No.: 19/563,449

(22) Filed: Mar. 11, 2026

Related U.S. Application Data
(60) Provisional application No. 63/770,134, filed on Mar. 11, 2025.

(51) Int. Cl. G01N 33/573; C07K 16/40; G01N 21/64; G01N 33/58

(52) U.S. Cl. CPC G01N 33/573; C07K 16/40; G01N 21/6486; G01N 33/582; C07K 2317/565; C07K 2317/622

(57) ABSTRACT

Described herein are Granzyme-B (GzmB) targeting imaging probes, methods of using, methods of making, and kits comprising probes described herein. In embodiments, the imaging probes comprise an anti-GzmB antibody or antigen binding fragment thereof. In embodiments, the anti-GzmB antibody or antigen binding fragment thereof is an scFV.

### CROSS-REFERENCE TO RELATED APPLICATION[S]

This application claims priority to, and the benefit of, U.S. Provisional Patent Application Ser. No. 63/770,134, entitled “GRANZYME B TARGETED IMAGING PROBES, KITS, METHOD OF MAKING, AND METHODS OF USE THEREOF” and filed on Mar. 11, 2025, the entire contents of which are incorporated herein by reference as if set forth in its entirety.

### SEQUENCE LISTING

The instant application contains a Sequence Listing which has been submitted electronically in .XML format and is hereby incorporated by reference in its entirety. Said .XML copy, created on Mar. 10, 2026, is named “222117_1820.xml” and is 17,592 bytes in size.

### BACKGROUND

Cytotoxic T lymphocytes (CTLs)-based immunotherapies, including checkpoint blockade, chimeric antigen receptor (CAR) T-cell, T-cell receptor (TCR) engineering, and tumor-infiltrating lymphocyte (TIL) therapies, have revolutionized the clinical management of cancer. Despite promising advancements, significant challenges remain, primarily revolving around the low rates of durable response. These challenges stem from inadequate T cell enrichment and expansion, limited ability to effectively reach tumor sites, lack of long-term persistence caused by T cell exhaustion within the immunosuppressive tumor microenvironment, and the immune escape of cancer cells, which facilitates resistance development. To overcome these limitations and enhance immunotherapy, there is a pressing need for new techniques that characterize T cell function in intact tissue or in vivo, and that trace and understand cancer cell escape and resistance mechanisms.

Molecular imaging has emerged as a promising approach for developing nondestructive and quantitative strategies to assess T cells. However, currently available T cell tracing methods, such as reporter transgenes, ex vivo T cell biolabeling, and CD8 immunoPET lack the ability to differentiate T cell functioning status. Furthermore, there is a notable lack of imaging tools capable of tracing T cell-engaged immune escaper cancer cells. Accordingly, there is a need to address the aforementioned deficiencies and inadequacies.

### SUMMARY

Described herein are Granzyme B (GzmB)-targeting probes (for imaging, for example) and compositions comprising such. In embodiments, described herein are composition comprising a Granzyme B (GzmB)-targeting imaging probe according to Formula 1 (N terminus to C terminus) X-G₃-P (Formula 1), wherein X comprises an anti-GzmB (aGzmB) antibody fragment; wherein G is glycine (G); and wherein P is a fluorochrome, caged fluorochrome, or radioisotope.

In embodiments, an imaging probe of the present disclosure comprises an aGzmB antibody fragment is an scFV. In embodiments, a aGzmB antibody or antibody fragment thereof comprises a CDRH1 polypeptide having at least 95% sequence identity to SEQ ID NO: 11; a CDRH2 polypeptide having at least 95% sequence identity to SEQ ID NO: 12; a CDRH3 polypeptide having at least 95% sequence identity to SEQ ID NO: 13; a CDRL1 polypeptide having at least 95% sequence identity to SEQ ID NO:14; a CDRL2 polypeptide having at least 95% sequence identity to SEQ ID NO: 15; and a CDRL3 polypeptide having at least 95% sequence identity to SEQ ID NO:16. In embodiments, imaging probes of the present disclosure comprise a aGzmB antibody fragment is an scFV that comprises: a VH polypeptide having at least 90% sequence identity to SEQ ID NO:17; and a VL polypeptide having at least 90% sequence identity to SEQ ID NO: 18.

In embodiments, the aGzmB antibody fragment comprises anti-human GzmB (ahGzmB) scFv S17 (SEQ ID NO:1). In embodiments, the aGzmB antibody fragment further comprises a 2×G4S tag (GGGGSGGGGS, SEQ ID NO:2), a Sort tag (LPETG, SEQ ID NO:3, or LPET, SEQ ID NO:7), or both, conjugated to the C-terminus of ahGzmB scFv S17 (SEQ ID NO: 1). In embodiments the aGzmB antibody fragment further comprises a pelB signal peptide (MKYLLPTAAAGLLLLAAQPAMA, SEQ ID NO:4), a S3FLAG tag (DYKDHDGDYKDHDIDYKDDDDK, SEQ ID NO: 5), or both, conjugated to the N-terminus of ahGzmB scFv S17 (SEQ ID NO:1). In embodiments, the anti-GzmB (aGzmB) antibody fragment (also referred to herein as “ahGzmB” or “AGB” comprises SEQ ID NO:6). In embodiments, the anti-GzmB (aGzmB) antibody fragment consists essentially of SEQ ID NO: 6. In embodiments, the anti-GzmB (aGzmB) antibody fragment consists of SEQ ID NO:6.

In embodiments, the fluorochrome is a near-infrared (NIR) cyanine dye, a hemicyanine dye, a coumarin-based dye, a fluorescein-based dye, a rhodamine-based dye, a BODIPY-Based Dye, a BODIPY-Based NIR Dye, a Xanthene-Based dye, a Xanthene-Based NIR Dye, a cyanine-based dyes, a porphyrin dye, a phthalocyanine dye, a squaraine dye. In embodiments, the fluorochrome comprises Cy5.5, Cy7, IRDye800CW, a Silicon Rhodamine, a Janelia Fluor 646, a Janelia Fluor 669, a Janelia Fluor 774); Squaraine Dyes, ICG, or IR-800.

In embodiments, the caged fluorochrome is a NIR caged cyanine or hemicyanine dye and is caged by a peptide that is capable of being cleaved or otherwise hydrolyzed by GzmB, for example, N′-IEPD-C′ (SEQ ID NO:8). In embodiment, the caged fluorochrome comprises CyP have a N′-IEPD-C′ moiety (SEQ ID NO:8).

In embodiment, the radioisotope comprises Zirconium-89 (⁸⁹Zr), Copper-64 (⁶⁴Cu) and Fluorine-18 (F-18), Technetium-99m, Iodine-131, Gallium-67, or Radium-223. In embodiments, the radioisotope comprises ⁸⁹Zr chelated with desferrioxamine (DFO) or GGG-DFO.

In embodiments, “G₃-P” has the following structure (N terminus to C terminus):

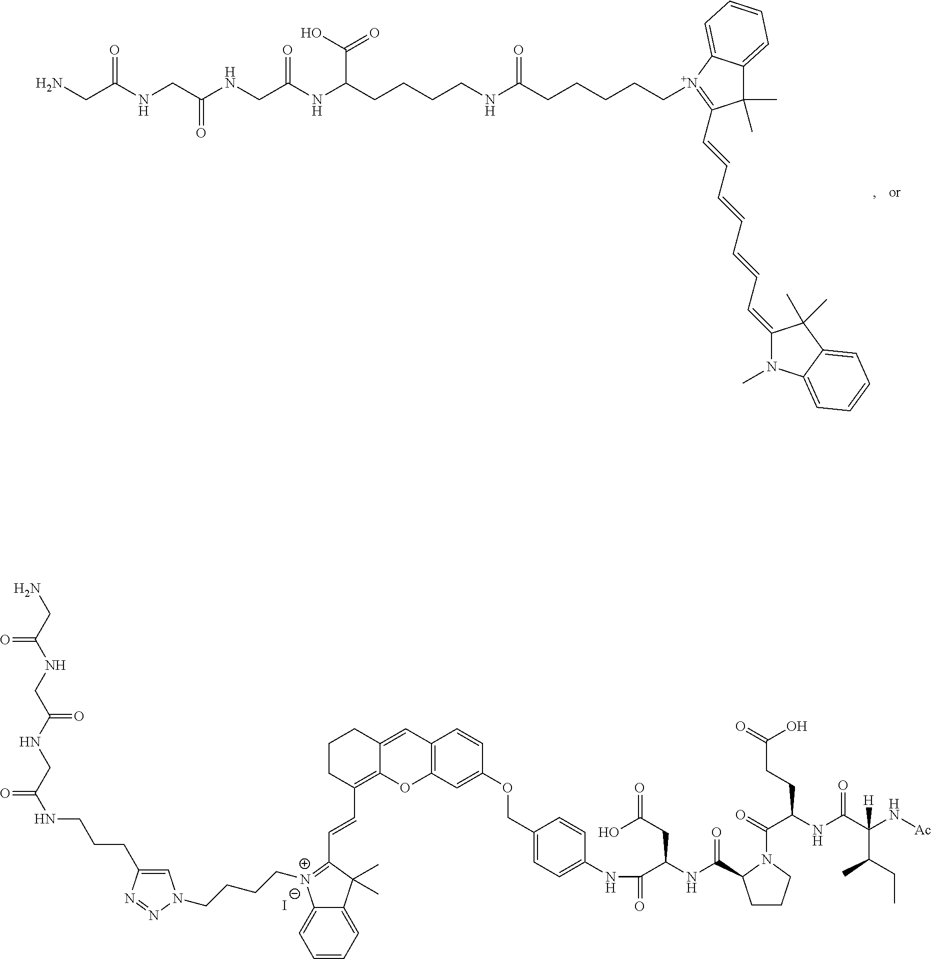 [SMILES: CN1/C(=C/C=C/C=C/C=C/C2=[N+](CCCCCC(=O)NCCCCC(NC(=O)CNC(=O)CNC(=O)CN)C(=O)O)c3ccccc3C2(C)C)C(C)(C)c2ccccc21 ; CC[C@@H](C)[C@@H](NC(C)=O)C(=O)N[C@H](CCC(=O)O)C(=O)N1CCC[C@H]1C(=O)N[C@H](CC(=O)O)C(=O)Nc1ccc(COc2ccc3c(c2)OC2=C(/C=C/C4=[N+](CCCCn5cc(CCCNC(=O)CNC(=O)CNC(=O)CN)nn5)c5ccccc5C4(C)C)CCCC2=C3)cc1 ; [I-]]

Described herein are methods of using Granzyme B (GzmB)-targeting imaging probes and compositions comprising such, in particular, for screening of suitable tissue fragments for TIL therapies.

In embodiments, a method of using a Granzyme B (GzmB)-targeting imaging probe, comprising contacting a sample with a composition as described herein; incubating the contacted sample at a temperature for a period of time; and imaging the incubated sample to determine a probe level.

In embodiments, sample is a tumor resection from a tumor of a subject or a tumor or suspected tumor in a subject.

In embodiments, the tumor is a metastatic melanoma, a sarcoma, a pancreatic tumor, an ovarian tumor, a colorectal tumor, a non-small cell lung cancer (NSCLC), and a triple-negative breast cancer tumor.

In embodiments, the subject is a subject undergoing or suspected to undergo tumor-infiltrating lymphocyte (TIL) therapy or has a metastatic or unresectable melanoma that has progressed on standard immune checkpoint inhibitors and BRAF-targeted therapies, a sarcoma, a pancreatic cancer, an ovarian cancer, a colorectal cancer, a non-small cell lung cancer, or a triple-negative breast cancer. In embodiments, the sample is incubated for about one hour at about 37° C.

In embodiments, methods further comprise identifying samples for use in future TIL therapy for the subject based on a determined probe level above a threshold. In embodiments, the determined probe level is a measured fluorescence intensity. In embodiments, the threshold is determined based on a measured fluorescence intensity in a control sample. In embodiments, the control sample is a non-tumor sample. In embodiments, the non-tumor sample is from the same subject as the tumor sample. In embodiments, the identified samples can comprise the top 25-50 percentage of tumor fragments of an individual subject ranked by fluorescent readout. Imaging probes, compositions, methods, and kits described herein can utilize detection modalities appropriate for a given fluorochrome, caged fluorochrome, or radioisotope known in the art.

Described herein are kits comprising probes as described herein. In embodiments, a kit can comprise any composition comprising any one or more probes (or multiple compositions comprising an individual probe in each composition, wherein each probe is diff from another) as described herein and instructions for use.

Described herein are methods of making Granzyme B-targeting probes as described herein, comprising executing any one or more chemical synthetic routes according to the present disclosure. Methods can further comprise isolating a compound produced by a synthetic route according to the present disclosure.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIGS. 1A-1B: (FIG. 1A) Illustration of CAR T cell degranulation and GzmB mediated cytotoxicity to kill cancer cells. (FIG. 1B) Crystal structure of assembled perforin pore in cancer cell membrane with 22 symmetry perforin monomers that allows influx of GzmB. (PMCID: PMC8836823).

FIGS. 2A-2B: Imaging GzmB mediated cytotoxicity (FIG. 2A) with fluorogenic AGB-CyP and fluorescence AGB-Cy7 probes (FIG. 2B).

FIGS. 3A-3B: design of an anti-human GzmB scFv (AGB) for expression and purification, compared to the native ahGzmB S17 sequence.

FIG. 4: Expression and purification of AGB. S-supernatant; F-flowthrough.

FIG. 5: Chemical structure and mass spectrum of GGG-Cy7 (M.W. 808).

FIG. 6: Synthesis of compound 2 to 7.

FIG. 7: Synthesis of compound 8 to 14.

FIG. 8: Mass spectrum of compound 14 (M.W. 1323).

FIGS. 9A-9C: Bio-labeling of AGB. (FIG. 9A) SrtA mediated bio-ligation of Cy7, CyP or a chelator with radioisotope to purified AGB. (FIG. 9B) SDS-PAGE showed the successful labeling and purification to generate AGB-Cy7. Ex-760/Em-780 (FIG. 9C) SDS-PAGE showed the successful labeling and purification to generate AGB-CyP. Ex650/Em690.

FIG. 10: Specific binding of AGB to GzmB in activated CD8 T cells. Flow cytometry analysis of CTLs showing comparable distribution with AGB-Cy7 and commercial GB11-RB545 fluorescence monoclonal antibody.

FIG. 11: Fluorescence imaging of GFP CD19-28ζ CAR T cells mediated cytotoxic killing of CD19+ U87 after 12 hours incubation in the presence of AGB-CyP (100 nM). GFP-green, Ex488/Em520; AGB-Cy-red, Ex690/Em710.

FIGS. 12A-12B: Characterization of AGB-Cy7 with GFP CD19 CAR T and Raji-RFP cells. (FIG. 12A) Flow cytometry analysis of GFP CD19 CAR T and Raji-RFP cells with or without AGB-Cy7. (FIG. 12B) Fluorescence imaging of the GFP CD19 CAR T and Raji-RFP cells in (FIG. 12A). RFP was greyed.

FIGS. 13A-13F: Establishment of a lymphoma spheroid CAR T-cell model. (FIG. 13A) Workflow to establish the spheroid model. (FIG. 13B) Assembly of Raji cells to spheroid. (FIG. 13C) Tumor infiltration of CD19 GFP-CAR T cells. (FIG. 13D) Killing efficiency of GFP-CAR T and CD8 T cells. (FIG. 13E) Tuning the effector: target cell ratio to induce T cell dysfunction. Scale bar-100 μm. (FIG. 13F) Flow cytometry analysis of CAR T cells within the lymphoma spheroid.

FIGS. 14A-14D: Characterization of AGB-CyP. (FIG. 14A) Normalized spectra of excitation-Em710. (FIG. 14B) Normalized spectra of emission-Ex 650. (FIG. 13C) Fluorescence turn-on of AGB-CyP at Ex690. (FIG. 13D) Kinetics of fluorescence turn on of GGG-CyP and AGB-CyP.

FIG. 15: Bioluminescence assay applying GFP CAR T cells to kill Raji-Fluc cells in the presence of AGB-CyP (200 nM). Effector-T cells; Target-Raji-Fluc.

FIG. 16: Fluorescence imaging of GFP CD19-28ζ CAR T cells infiltrate and kill Raji-RFP tumor spheroids. The cell numbers were quantified immediately after fluorescence imaging.

FIG. 17: Imaging TILs in 0.2-0.3 grams tissue phantom. Red-AGB-CyP, Ex680.

FIG. 18: AGB-89Zr tracer PET imaging tracer.

FIG. 19: Illustration of the three key steps in TIL therapy.

FIG. 20: The fluorogenic screening assay incorporated in step 1 enables rapid and specific identification of functional TILs within fragmented tumor resections.

FIG. 21: Illustration of CAR T cell mediated cytotoxicity to kill target cell.

FIGS. 22A-22B: Design of AGB-CyP. (FIG. 22A) Activation and GzmB binding of AGB-CyP. (FIG. 22B) Fluorescence labeling of CTLs engaged cancer cell.

FIGS. 23A-23E: Synthesis and characterization of AGB-CyP. (FIG. 23A) SrtA mediated bio-ligation. (FIG. 23B) SimpleBlue and fluorescence (Ex650/Em690) gel scan. Rx-labeling reaction. F.T.-flowthrough. (FIG. 23C) Flow cytometry analysis of CTLs showing comparable distribution with AGB-Cy7 and commercial GB11-RB545 fluorescence monoclonal antibody. (FIG. 23D) Fluorescence turn-on at Ex690. (FIG. 22E) Kinetics of fluorescence turn on of AGB-CyP and G3-CyP.

FIGS. 24A-24B: Cell study of AGB-CyP. (FIG. 22A) Fluorescence imaging of GFP CD19-28ζ CAR T cells mediated cytotoxic killing of CD19+ U87 after 12 hours incubation in the presence of AGB-CyP (100 nM). GFP-green, Ex488/Em520; AGB-Cy-red, Ex690/Em710. (FIG. 22B) Bioluminescence assay applying GFP CAR T cells to kill Raji-Fluc cells in the presence of AGB-CyP (200 nM). Effector-T cells; Target-Raji-Fluc.

FIG. 25: Fluorescence imaging of GFP CD19-28ζ CAR T cells infiltrate and kill Raji-RFP tumor spheroids.

FIG. 26: Imaging TILs in 0.2-0.3 grams tissue phantom. Red-AGB-CyP, Ex680.

FIG. 27A-27B: Induction of CAR T cell exhaustion in tumor spheroid models. (FIG. 27A) Killing of lymphoma spheroid (Raji-RFP, red) by GFP CAR T cells (green). Scale bar-500 μm. (FIG. 27B) Upregulation of T cell exhaustion markers after 7-day tumor antigen exposure (1:8).

FIGS. 28A-28D Granzyme B is highly distributed in CTL-engaged tumor cells. (FIG. 28A) Representative immunofluorescence staining of FFPE (formalin-fixed paraffin-embedded) surgical melanoma resections. CD8a-FITZ (green), Melan-A-Cy5 (red), GzmB-Trtz (yellow), DAPI (blue). (FIG. 28B) Percentage distribution of GzmB fluorescence intensity in tumor cells (Melan-A+), cytotoxic T lymphocytes (CD8a+), and ECM (extracellular matrix, Melan-A−, CD8a−). Error bars represent SD. (FIG. 28C) The concentration of GzmB fluorescence in tumor cells, ECM and CD8 T cells. Error bars represent SD. (FIG. 28D) Percentage distribution of GzmB fluorescence intensity in each patient having two or more independent tumor regions identified. The mean values were indicated.

FIGS. 29A-29G Strategy for fluorescence detection of degranulated GzmB. (FIG. 29A) Mechanism of GzmB detection by AGB-CyP upon CTLs engaging and killing cancer cells. (FIG. 29B) Comparison of human (purpal) and mouse (orange) perforin monomer structures. (FIG. 29C) Simulated human perforin pore with C21 symmetry. (FIG. 29D) Predicted structure of human GzmB/AGB-Cy complex. The catalytic pocket is defined by His57, Asp102, Lys192, Gly193, Asp194, Ser195, Ser214, Gly216, Asn218, Arg226 and is colored in pink. (FIG. 29E) Synthesis of triglycine-CyP (G₃-CyP) for sortase A mediated bio-ligation with recombinant AGB-SUMO to generate AGB-CyP. (FIG. 29F) Simpleblue staining (left) and fluorescent (right, Ex650/Em690) scan of SDS-PAGE gel analyzing AGB-SUMO, sortase A (StA), reaction with G₃-CyP (Rx.), and the final reverse affinity purification (F.T.—flowthrough) product AGB-CyP. (FIG. 29G) Cleavage of IEPD (SEQ ID NO:8) by GzmB triggers the leaving of the self-imolative linker to turn on fluorescence and in turn the binding of the uncaged AGB-Cy probe to GzmB.

FIGS. 30A-30G. AGB-CyP detects GzmB in CTLs engaged tumor cells. (FIG. 30A) Flow Cytometry analysis of GzmB in permeabilized resting and activated CD8+ T cells with AGB-Cy7 and GB11-RB545 antibody. (FIG. 30B) Normalized absorption spectra and (FIG. 30C) emission spectra of AGB-CyP (10 μM) in the absence or presence of human GzmB (2.5 ng/μL) in Tris buffer (50 mM, pH 7.5) at 37° C. (FIG. 30D) Fluorescence turn-on of AGB-CyP (10 μM) by GzmB (Ex690, 58-fold at Em717). (FIG. 30E) Enzymatic kinetics of AGB-CyP in the absence or presence of GzmB and AGB pretreated GzmB. The curves represent nonlinear fitting. (FIG. 30F) Representative confocal fluorescence images of GFP expressing CD19-28ζ CAR T cells mediated cytotoxic killing of RFP expressing CD19+ U87 malignant glioma cells after 12 hours incubation in the presence of AGB-CyP (100 nM). Scalebar-10 μm. (FIG. 30G) Flow Cytometry analysis of Raji lymphoma cells treated with or without CD19-282 CAR T cells.

FIGS. 31A-31J. AGB-CyP sensitively detects functional, cancer-reactive TILs in lymphoma spheroids. (FIG. 31A) Workflow to establish the lymphoma spheroid CD19-28ζ CAR T cell model. (FIG. 31B) Assembly of 5×10⁵ Raji cells to a lymphoma spheroid. Scalebar-250 μm. (FIG. 31C) Growth curves of suspended Raji cells vs spheroids. Error bars represent SD. (FIG. 31D) Longitudinal epifluorescence imaging of GFP CD19-28ζ CAR T cells infiltrating a lymphoma spheroid. Scalebar-250 μm. (FIG. 31E) Bioluminescence assay applying GFP CAR T cells to kill Raji-Fluc cells for 24 hours. Effector-T cells; Target-Raji-Fluc. Error bars represent SD. (FIG. 31F) Flow Cytometry analysis of TILs in lymphoma spheroids. (FIG. 31G) Representative epifluorescence images showing AGB-CyP detects 10³-10⁵ functional TILs in lymphoma spheroids. Scalebar-650 μm. (FIG. 31H) Representative epifluorescence images showing AGB-CyP differentiates functional from terminally exhausted TILs. Scalebar-650 μm. (FIG. 31I) Quantification of the total fluorescence of spheroids in (FIG. 31H). (FIG. 31J) Confocal fluorescence images of spheroids treated with resting, CD3/28 beads activated or terminally exhausted CAR T cells in (FIG. 31H). Scalebar-50 μm.

FIGS. 32A-32G. AGB-CyP detects surgical tumor resections containing functional TILs. (FIG. 31A) Bright-field and H&E staining of an OCT media embedded frozen melanoma tumor resection. (FIG. 31B) Multiplex fluorescence staining of a tissue slide generated from the sample. Scalebar-2 mm. (FIG. 31C) Macrodissection of the frozen tissue to generate tumor and adjacent non-malignant tissue fragments for AGB-CyP staining and imaging. Ruler scale-1 mm. (FIG. 31D) Representative bright-field and fluorescence imaging (Ex690/Em715) of tissue fragments after 7-hour AGB-CyP (200 nM) treatment. Scalebar-1 mm. (FIG. 31E) Mean fluorescence (AU-arbitrary units) of tissue fragments in Dulbecco's phosphate-buffered saline (DPBS) before probe treatment. Significance was determined using unpaired t test, **p<0.0022. (FIG. 31F) Longitudinal quantification of percentage mean fluorescence increase of tissue fragments upon AGB-CyP treatment. The curves represent nonlinear fitting. (FIG. 31G) Tumor fragments exhibited significant higher mean fluorescence than adjacent non-malignant tissues after 2 hours probe incubation. Error bars represent SD. Significance was determined using unpaired t test, ****p<0.0001.

FIG. 33 shows generation of a AGB-Cy7 probe according to the present disclosure.

FIG. 34 is a graph showing AGB-CyP stability in culture media.

FIG. 35 is a graph showing AGB-CyP has no impact on CAR T cell viability and cytotoxic function.

FIG. 36 Induction of CAR T cell dysfunction in lymphoma spheroids. FIG. 36 shows adjusting the effector (CAR T cells): Target (Raji) cell ratio to induce cell dysfunction. Scale bar=650 μM.

FIG. 37 displays results of a flow cytometry analysis of perforin in TILs.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Regarding machine hardware, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally remotely any and/or with user/operator/customer/client or machine/system/computer/processor. Moreover, the various components may be in communication via wireless and/or hardwire or other desirable and available communication means, systems and hardware. Moreover, various components and modules may be substituted with other modules or components that provide similar functions.

Embodiments of the present disclosure will employ, unless otherwise indicated, techniques of medicinal chemistry, biochemistry, molecular biology, cellular biology, tissue culture, therapeutic administrations, cancer biology, cancer therapies, cancer diagnostics and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, ±4%, ±3%, ±2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters. In embodiments the control sample is a sample from a subject not having a tumor, or a non-tumor sample from the same subject as the tumor sample.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and also includes cells in cell culture, cell supernatants, cell lysates, serum, plasma, biological fluids, and tissue samples.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or more higher.

The terms “administering,” “delivering,” and “introducing,” can be used interchangeably to indicate the introduction of a therapeutic composition or agent (e.g., compositions comprising one or more bacterial species as described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the therapeutic include, but are not limited to, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a 24-hour period.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, CRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, CRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct. The term polypeptide as used herein can also refer to a sequence that is a portion of a larger polypeptide sequence.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23 (4): 581-87 (2013); Xie et al. “Adding amino acids to the genetic repertoire,” Curr. Opin. Chem. Biol. 9 (6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-10 and Altschul et al. (1977) Nucleic Acids Res. 25:3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

As used herein, the term “antibody” encompasses antibodies and antibody fragments thereof, derived from any antibody-producing mammal (e.g., mouse, rat, rabbit, and primate including human), or from a hybridoma, phage selection, recombinant expression or transgenic animals (or other methods of producing antibodies or antibody fragments”), that specifically bind to a target polypeptide, such as, for example, Granzyme B, polypeptides or portions thereof. It is not intended that the term “antibody” limited as regards to the source of the antibody or the manner in which it is made (e.g., by hybridoma, phage selection, recombinant expression, transgenic animal, peptide synthesis, etc). Exemplary antibodies include polyclonal, monoclonal and recombinant antibodies; pan-specific, multispecific antibodies (e.g., bispecific antibodies, trispecific antibodies); humanized antibodies; murine antibodies; chimeric, mouse-human, mouse-primate, primate-human monoclonal antibodies; and anti-idiotype antibodies and may be any intact antibody or fragment thereof. As used herein, the term “antibody” encompasses not only intact polyclonal or monoclonal antibodies, but also fragments thereof (such as dAb, Fab, Fab′, F(ab′)2, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific for the target antigen. The term “monoclonal antibody” encompasses not only intact monoclonal antibodies and full-length monoclonal antibodies, but also fragments thereof (such as Fab, Fab′, F(ab′) 2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”. As used herein, the term “antibody fragment” refers to a portion derived from or related to a full-length antibody, such as, for example, an anti-GOLIM4 antibody, generally including the antigen binding or variable region thereof. Illustrative examples of antibody fragments include Fab, Fab′, F (ab) 2, F(ab′) 2 and Fv fragments, scFv fragments, diabodies, linear antibodies, single-chain antibody molecules and multispecific antibodies formed from antibody fragments. As used herein, a “single-chain Fv” or “scFv” antibody fragment comprises the VH and VL domains of an antibody, wherein these domains are present in a single polypeptide chain. Generally, the Fv polypeptide further comprises a polypeptide linker between the VH and VL domains, which enables the scFv to form the desired structure for antigen binding. As used herein, a “chimeric antibody” is a recombinant protein that contains the variable domains and complementarity-determining regions derived from a non-human species (e.g., rodent) antibody, while the remainder of the antibody molecule is derived from a human antibody.

### DISCUSSION

Described herein are novel Granzyme B-targeting probes, methods of use, methods of making, and kits comprising such.

In certain aspects, described herein are novel approaches that involve monitoring T cell function and tracing escaped cancer cells through imaging degranulated granzyme B (GzmB), using specially designed GzmB-activated single-chain variable fragment (scFv) imaging probes described herein.

In certain aspects, the design, synthesis, and characterization of GzmB targeting scFv fluorescence probes is described herein, as well as the design of PET tracers for applications including those at least listed below:
1) identification of functional cytotoxic T lymphocytes (CTLs) through optical imaging and positron emission tomography in 2D cell culture in vitro, 3D tumor spheroids in vitro, or intact live tissue ex vivo or in vivo;
2) tracing CTL-engaged cancer cells, either apoptotic or viable escaper, to identify new genes and mechanisms involved in therapeutic resistance and immune evasion in various experimental models and humans; and
3) predicting the therapeutic response to CTLs and CTL modulating immunotherapies.

Such aspects of the present disclosure can provide novel imaging tools and methodologies utilized such that are capable, for example, of tracing T cell-engaged immune escaper cancer cells.

### I. Granzyme B Targeted Imaging Probes

Described herein are novel Granzyme B (GzmB)-targeting probes. Such probes can be used, for example, for identifying T cell function and tracing escaped cancer cells through imaging degranulated granzyme B (GzmB). In certain aspects, probes as described herein can utilize specially designed GzmB-activated single-chain variable fragment (scFv) imaging probes described herein, for example. Compositions comprising imaging probes according to the present disclosure are also contemplated, for example, comprising one or more imaging probes according to the present disclosure and an aqueous or otherwise pharmaceutically acceptable vehicle.

Imaging probes as described herein can utilize an antibody fragment or binding portion thereof to selectively bind to Granzyme B. anti-GzmB antibody fragments as described herein can be conjugated, for example, at their C-terminus to the N-terminus of a G₃ (Gly-Gly-Gly) tagged probe (for example, a fluorochrome, caged fluorochrome, or radioisotope for PET imaging) through a synthetic technique such as Sortase A-mediated bioligation as further described herein.

In embodiments, described herein are composition comprising a Granzyme B (GzmB)-targeting imaging probe according to Formula 1 (N terminus to C terminus) X-G₃-P (Formula 1), wherein X comprises an anti-GzmB (aGzmB) antibody fragment; wherein G is glycine (G); and wherein P is a fluorochrome, caged fluorochrome, or radioisotope. In embodiments, the aGzmB antibody fragment comprises anti-human GzmB (ahGzmB) scFv S17 (Genbank accession no. AY574385, which is incorporated by reference in its entirety as if fully set forth herein). In embodiments, the aGzmB antibody fragment comprises anti-human GzmB (ahGzmB) scFv S17 (SEQ ID NO:1) or antigen binding fragment [s] thereof. In embodiments, the aGzmB antibody fragment further comprises a 2×G4S tag (GGGGSGGGGS, SEQ ID NO: 2), a Sort tag (LPETG, SEQ ID NO:3, or LPET, SEQ ID NO:7), or both, conjugated to the C-terminus of ahGzmB scFv S17 (SEQ ID NO:1). In embodiments the aGzmB antibody fragment further comprises a pelB signal peptide (MKYLLPTAAAGLLLLAAQPAMA, SEQ ID NO: 4), a S3FLAG tag (DYKDHDGDYKDHDIDYKDDDDK, SEQ ID NO: 5), or both, conjugated to the N-terminus of ahGzmB scFv S17 (SEQ ID NO:1). In embodiments, the anti-GzmB (aGzmB) antibody fragment (also referred to herein as “ahGzmB” or “AGB” comprises SEQ ID NO: 6). In embodiments, the anti-GzmB (aGzmB) antibody fragment consists essentially of SEQ ID NO:6. In embodiments, the anti-GzmB (aGzmB) antibody fragment consists of SEQ ID NO: 6.

### A. Granzyme B-Targeting Antibody Fragments

i. Granzyme B-Targeting Antibody Fragments

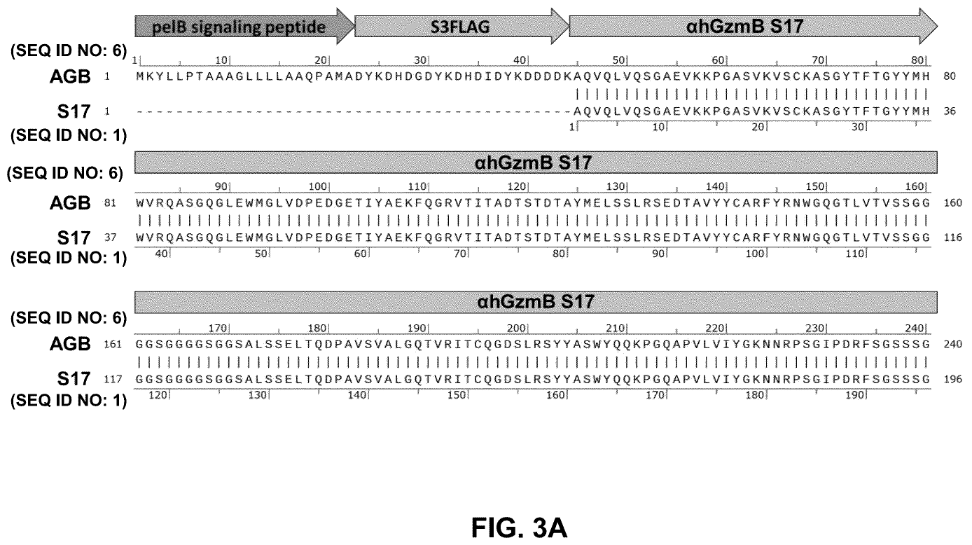

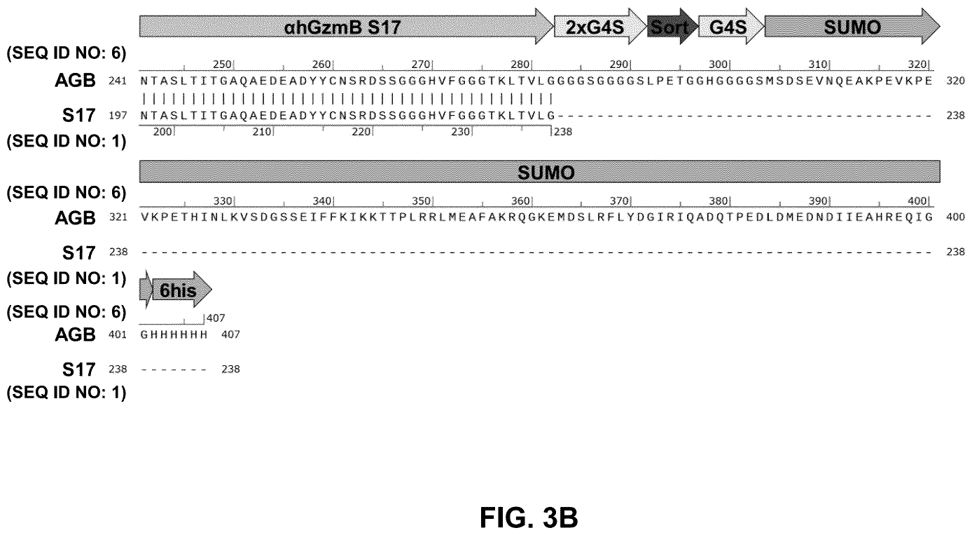

Antibodies or antigen binding portions thereof (also referred to herein as “antigen binding fragments” thereof) that specifically or selectively binds GzmB (or antigens thereof, in particular antigens that may retain the GzmB catalytic activity) are provided herein. In certain aspects, antibodies as described herein are human or rodent antibody fragments. In embodiments of the present disclosure, novel antibodies (or antibody fragments) specifically bind to GzmB as a tumor antigen. Embodiments of such comprise those shown in FIGS. 3A-3B and in the informal sequence listing below. In embodiments, the anti-GzmB antibody fragments described herein can be an scFV.

As used herein, the terms specifically binds to, specific for, selectively binds and selective for GzmB or a GzmB antigen means binding that is measurably different from a nonspecific or non-selective interaction. Specific binding can be measured, for example, by determining binding of a molecule compared to binding of a control molecule. Specific binding can also be determined by competition with a control molecule that is similar to the target, such as an excess of non-labeled target. In that case, specific binding is indicated if the binding of the labeled target to a probe is competitively inhibited by the excess non-labeled target.

An antibody, as used herein, can refer to an intact antibody (e.g., an intact immunoglobulin) or an antibody fragment, for example, an antigen binding fragment. Antigen binding fragments can comprise at least one antigen binding domain. One example of an antigen binding domain is an antigen binding domain formed by a V_{H}-V_{L} dimer. Antibodies and antigen binding fragments can be described by the antigen to which they specifically bind.

The V_{H} and V_{L} regions can be further subdivided into regions of hypervariability (hypervariable regions (HVRs), also called complementarity determining regions (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding and confer antigen specificity and binding affinity to the antibody. (See Kabat et al. (1991) Sequences of Proteins of Immunological Interest 5th ed., Public Health Service, National Institutes of Health, Bethesda, MD.) CDR sequences on the heavy chain (VH) may be designated as CDRH1, 2, 3, while CDR sequences on the light chain (Vv) may be designated as CDRL1, 2, 3.

The term epitope, as used herein, means a component of an antigen (such as a GzmB or catalytically-active fragment thereof) capable of specific binding to an antibody or antigen binding fragment thereof. Such components optionally comprise one or more contiguous amino acid residues and/or one or more non-contiguous amino acid residues. Epitopes frequently consist of surface-accessible amino acid residues and/or sugar side chains and can have specific three-dimensional structural characteristics, as well as specific charge characteristics. Conformational and non-conformational epitopes are distinguished in that the binding to the former but not the latter is lost in the presence of denaturing solvents. An epitope can comprise amino acid residues that are directly involved in the binding, and other amino acid residues, which are not directly involved in the binding. The epitope to which an antigen binding protein binds can be determined using known techniques for epitope determination such as, for example, testing for antigen binding protein binding to antigen variants with different point mutations.

Provided herein are antibodies or antigen binding portions thereof that specifically bind to antigens related to GzmB, in particular GzmB and catalytically active fragments thereof. Such antibodies may be, for example, monoclonal antibodies (mAbs) or recombinant/chimeric antibodies (i.e., synthetic antibodies derived from synthetic nucleic acid constructs, such as viral vectors, that may also contain monoclonal Ab sequences as disclosed herein).

The present disclosure provides an antibody or antigen binding portion thereof that specifically binds to a GzmB having CDRH1, CDRH2, CDRH3, CDRL1, CDRL2, and CDRL3 sequences.

In embodiments, an anti-GzmB antibody or antigen binding portion comprises a CDRH1 comprising an amino sequencing having at least 75% to 85 to 90% or greater (e.g. 90%, 91%, 92%, 93%, 94%, 95%, 96%, 97%, 98%, or 99%) identity to SEQ ID NO: 11. In embodiments, an anti-GzmB antibody or antigen binding portion comprises a CDRH1 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 11. In embodiments, an anti-GzmB antibody or antigen binding portion comprises a CDRH1 consisting of an amino sequencing having at least 75% to 85 to 90% or greater (e.g. 90%, 91%, 92%, 93%, 94%, 95%, 96%, 97%, 98%, or 99%) identity to SEQ ID NO: 11.

In embodiments, an anti-GzmB antibody or antigen binding portion comprises a CDRH2 comprising an amino sequencing having at least 75% to 85 to 90% or greater (e.g. 90%, 91%, 92%, 93%, 94%, 95%, 96%, 97%, 98%, or 99%) identity to SEQ ID NO: 12. In embodiments, an anti-GzmB antibody or antigen binding portion comprises a CDRH2 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 12. In embodiments, an anti-GzmB antibody or antigen binding portion comprises a CDRH2 consisting of an amino sequencing having at least 75% to 85 to 90% or greater (e.g. 90%, 91%, 92%, 93%, 94%, 95%, 96%, 97%, 98%, or 99%) identity to SEQ ID NO: 12.

In embodiments, an anti-GzmB antibody or antigen binding portion comprises a CDRH3 comprising an amino sequencing having at least 75% to 85 to 90% or greater (e.g. 90%, 91%, 92%, 93%, 94%, 95%, 96%, 97%, 98%, or 99%) identity to SEQ ID NO: 13. In embodiments, an anti-GzmB antibody or antigen binding portion comprises a CDRH3 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 13. In embodiments, an anti-GzmB antibody or antigen binding portion comprises a CDRH3 consisting of an amino sequencing having at least 75% to 85 to 90% or greater (e.g. 90%, 91%, 92%, 93%, 94%, 95%, 96%, 97%, 98%, or 99%) identity to SEQ ID NO: 13.

In embodiments, an anti-GzmB antibody or antigen binding portion comprises a CDRL1 comprising an amino sequencing having at least 75% to 85 to 90% or greater (e.g. 90%, 91%, 92%, 93%, 94%, 95%, 96%, 97%, 98%, or 99%) identity to SEQ ID NO: 14. In embodiments, an anti-GzmB antibody or antigen binding portion comprises a CDRL1 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 14. In embodiments, an anti-GzmB antibody or antigen binding portion comprises a CDRL1 consisting of an amino sequencing having at least 75% to 85 to 90% or greater (e.g. 90%, 91%, 92%, 93%, 94%, 95%, 96%, 97%, 98%, or 99%) identity to SEQ ID NO: 14.

In embodiments, an anti-GzmB antibody or antigen binding portion comprises a CDRL2 comprising an amino sequencing having at least 75% to 85 to 90% or greater (e.g. 90%, 91%, 92%, 93%, 94%, 95%, 96%, 97%, 98%, or 99%) identity to SEQ ID NO: 15. In embodiments, an anti-GzmB antibody or antigen binding portion comprises a CDRL2 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 15. In embodiments, an anti-GzmB antibody or antigen binding portion comprises a CDRL2 consisting of an amino sequencing having at least 75% to 85 to 90% or greater (e.g. 90%, 91%, 92%, 93%, 94%, 95%, 96%, 97%, 98%, or 99%) identity to SEQ ID NO: 15.

In embodiments, an anti-GzmB antibody or antigen binding portion comprises a CDRL3 comprising an amino sequencing having at least 75% to 85 to 90% or greater (e.g. 90%, 91%, 92%, 93%, 94%, 95%, 96%, 97%, 98%, or 99%) identity to SEQ ID NO: 16. In embodiments, an anti-GzmB antibody or antigen binding portion comprises a CDRL3 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 16. In embodiments, an anti-GzmB antibody or antigen binding portion comprises a CDRL3 consisting of an amino sequencing having at least 75% to 85 to 90% or greater (e.g. 90%, 91%, 92%, 93%, 94%, 95%, 96%, 97%, 98%, or 99%) identity to SEQ ID NO: 16.

In embodiments, an anti-GzmB antibody or antigen binding portion comprises: a CDRH1 comprising an amino sequencing having at least 75% to 85 to 90% or greater (e.g. 90%, 91%, 92%, 93%, 94%, 95%, 96%, 97%, 98%, or 99%) identity to SEQ ID NO: 11; a CDRH2 comprising an amino sequencing having at least 75% to 85 to 90% or greater (e.g. 90%, 91%, 92%, 93%, 94%, 95%, 96%, 97%, 98%, or 99%) identity to SEQ ID NO: 12; a CDRH3 comprising an amino sequencing having at least 75% to 85 to 90% or greater (e.g. 90%, 91%, 92%, 93%, 94%, 95%, 96%, 97%, 98%, or 99%) identity to SEQ ID NO: 13; a CDRL1 comprising an amino sequencing having at least 75% to 85 to 90% or greater (e.g. 90%, 91%, 92%, 93%, 94%, 95%, 96%, 97%, 98%, or 99%) identity to SEQ ID NO: 14; a CDRL2 comprising an amino sequencing having at least 75% to 85 to 90% or greater (e.g. 90%, 91%, 92%, 93%, 94%, 95%, 96%, 97%, 98%, or 99%) identity to SEQ ID NO: 15; and a CDRL3 comprising an amino sequencing having at least 75% to 85 to 90% or greater (e.g. 90%, 91%, 92%, 93%, 94%, 95%, 96%, 97%, 98%, or 99%) identity to SEQ ID NO: 16.

In embodiments, an anti-GzmB antibody or antigen binding portion comprises: a

CDRH1 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 11; a CDRH2 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 12; a CDRH3 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 13; a CDRL1 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 14; a CDRL2 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 15; and a CDRL3 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 16.

In embodiments, an anti-GzmB antibody or antigen binding portion comprises: a CDRH1 consisting of an amino sequencing having at least 75% to 85 to 90% or greater (e.g. 90%, 91%, 92%, 93%, 94%, 95%, 96%, 97%, 98%, or 99%) identity to SEQ ID NO: 11; a CDRH2 consisting of an amino sequencing having at least 75% to 85 to 90% or greater (e.g. 90%, 91%, 92%, 93%, 94%, 95%, 96%, 97%, 98%, or 99%) identity to SEQ ID NO: 12; a CDRH3 consisting of an amino sequencing having at least 75% to 85 to 90% or greater (e.g. 90%, 91%, 92%, 93%, 94%, 95%, 96%, 97%, 98%, or 99%) identity to SEQ ID NO: 13; a CDRL1 consisting of an amino sequencing having at least 75% to 85 to 90% or greater (e.g. 90%, 91%, 92%, 93%, 94%, 95%, 96%, 97%, 98%, or 99%) identity to SEQ ID NO: 14; a CDRL2 consisting of an amino sequencing having at least 75% to 85 to 90% or greater (e.g. 90%, 91%, 92%, 93%, 94%, 95%, 96%, 97%, 98%, or 99%) identity to SEQ ID NO: 15; and a CDRL3 consisting of an amino sequencing having at least 75% to 85 to 90% or greater (e.g. 90%, 91%, 92%, 93%, 94%, 95%, 96%, 97%, 98%, or 99%) identity to SEQ ID NO: 16.

In embodiments, an anti-GzmB antibody or antigen binding portion comprises: a CDRH1 comprising an amino sequencing having at least 75% to 85 to 90% or greater (e.g. 90%, 91%, 92%, 93%, 94%, 95%, 96%, 97%, 98%, or 99%) identity to SEQ ID NO: 11; a CDRH2 comprising an amino sequencing having at least 75% to 85 to 90% or greater (e.g. 90%, 91%, 92%, 93%, 94%, 95%, 96%, 97%, 98%, or 99%) identity to SEQ ID NO: 12; a CDRH3 comprising an amino sequencing having at least 75% to 85 to 90% or greater (e.g. 90%, 91%, 92%, 93%, 94%, 95%, 96%, 97%, 98%, or 99%) identity to SEQ ID NO: 13. In embodiments, an anti-GzmB antibody or antigen binding portion comprises: a CDRH1 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 11; a CDRH2 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 12; a CDRH3 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 13. In embodiments, an anti-GzmB antibody or antigen binding portion comprises: a CDRH1 consisting of an amino sequencing having at least 75% to 85 to 90% or greater (e.g. 90%, 91%, 92%, 93%, 94%, 95%, 96%, 97%, 98%, or 99%) identity to SEQ ID NO: 11; a CDRH2 consisting of an amino sequencing having at least 75% to 85 to 90% or greater (e.g. 90%, 91%, 92%, 93%, 94%, 95%, 96%, 97%, 98%, or 99%) identity to SEQ ID NO: 12; a CDRH3 consisting of an amino sequencing having at least 75% to 85 to 90% or greater (e.g. 90%, 91%, 92%, 93%, 94%, 95%, 96%, 97%, 98%, or 99%) identity to SEQ ID NO: 13.

In embodiments, an anti-GzmB antibody or antigen binding portion comprises: a CDRL1 comprising an amino sequencing having at least 75% to 85 to 90% or greater (e.g. 90%, 91%, 92%, 93%, 94%, 95%, 96%, 97%, 98%, or 99%) identity to SEQ ID NO: 14; a CDRL2 comprising an amino sequencing having at least 75% to 85 to 90% or greater (e.g. 90%, 91%, 92%, 93%, 94%, 95%, 96%, 97%, 98%, or 99%) identity to SEQ ID NO: 15; and a CDRL3 comprising an amino sequencing having at least 75% to 85 to 90% or greater (e.g. 90%, 91%, 92%, 93%, 94%, 95%, 96%, 97%, 98%, or 99%) identity to SEQ ID NO: 16. In embodiments, an anti-GzmB antibody or antigen binding portion comprises: a CDRL1 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 14; a CDRL2 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 15; and a CDRL3 consisting essentially of an amino sequencing having at least 75% to 85 to 90% or greater (e.g. 90%, 91%, 92%, 93%, 94%, 95%, 96%, 97%, 98%, or 99%) identity to SEQ ID NO: 16. In embodiments, an anti-GzmB antibody or antigen binding portion comprises: a CDRL1 consisting of an amino sequencing having at least 75% to 85 to 90% or greater (e.g. 90%, 91%, 92%, 93%, 94%, 95%, 96%, 97%, 98%, or 99%) identity to SEQ ID NO: 14; a CDRL2 consisting of an amino sequencing having at least 75% to 85 to 90% or greater (e.g. 90%, 91%, 92%, 93%, 94%, 95%, 96%, 97%, 98%, or 99%) identity to SEQ ID NO: 15; and a CDRL3 consisting of an amino sequencing having at least 75% to 85 to 90% or greater (e.g. 90%, 91%, 92%, 93%, 94%, 95%, 96%, 97%, 98%, or 99%) identity to SEQ ID NO: 16.

In embodiments, an anti-GzmB antibody or antigen binding portion comprises: a CDRH1 comprising SEQ ID NO: 11; a CDRH2 comprising SEQ ID NO: 12; a CDRH3 comprising a SEQ ID NO: 13; a CDRL1 comprising SEQ ID NO: 14; a CDRL2 comprising SEQ ID NO: 15; and a CDRL3 comprising SEQ ID NO: 16.

In embodiments, an anti-GzmB antibody or antigen binding portion comprises: a CDRH1 consisting essentially of SEQ ID NO: 11; a CDRH2 consisting essentially of an SEQ ID NO: 12; a CDRH3 consisting essentially of SEQ ID NO: 13; a CDRL1 consisting essentially of a SEQ ID NO: 14; a CDRL2 consisting essentially of SEQ ID NO: 15; and a CDRL3 consisting essentially of a SEQ ID NO: 16.

In embodiments, an anti-GzmB antibody or antigen binding portion comprises: a CDRH1 consisting of SEQ ID NO: 11; a CDRH2 consisting of SEQ ID NO: 12; a CDRH3 consisting of SEQ ID NO: 13; a CDRL1 consisting of SEQ ID NO: 14; a CDRL2 consisting of SEQ ID NO: 15; and a CDRL3 consisting of SEQ ID NO: 16.

In embodiments, an anti-GzmB antibody or antigen binding portion comprises: a CDRH1 comprising SEQ ID NO: 11; a CDRH2 comprising SEQ ID NO: 12; and a CDRH3 comprising a SEQ ID NO: 13. In embodiments, an anti-GzmB antibody or antigen binding portion comprises: a CDRH1 consisting essentially of SEQ ID NO: 11; a CDRH2 consisting essentially of an SEQ ID NO: 12; and a CDRH3 consisting essentially of SEQ ID NO: 13. In embodiments, an anti-GzmB antibody or antigen binding portion comprises: a CDRH1 consisting of SEQ ID NO: 11; a CDRH2 consisting of SEQ ID NO: 12; and a CDRH3 consisting of SEQ ID NO: 13.

In embodiments, an anti-GzmB antibody or antigen binding portion comprises: a CDRL1 comprising SEQ ID NO: 14; a CDRL2 comprising SEQ ID NO: 15; and a CDRL3 comprising SEQ ID NO: 16. In embodiments, an anti-GzmB antibody or antigen binding portion comprises: a CDRL1 consisting essentially of a SEQ ID NO: 14; a CDRL2 consisting essentially of SEQ ID NO: 15; and a CDRL3 consisting essentially of a SEQ ID NO: 16. In embodiments, an anti-GzmB antibody or antigen binding portion comprises: a CDRL1 consisting of SEQ ID NO: 14; a CDRL2 consisting of SEQ ID NO: 15; and a CDRL3 consisting of SEQ ID NO: 16.

In each case, where a specific amino acid sequence is recited, embodiments comprising a VH domain comprising, consisting essentially of, or consisting of at least 75% to 85 to 90% or greater (e.g. 90%, 91%, 92%, 93%, 94%, 95%, 96%, 97%, 98%, 99%, or 100%) identity to SEQ ID NO: 17 are also provided.

In each case, where a specific amino acid sequence is recited, embodiments comprising a VL domain comprising, consisting essentially of, or consisting of at least 75% to 85 to 90% or greater (e.g. 90%, 91%, 92%, 93%, 94%, 95%, 96%, 97%, 98%, 99%, or 100%) identity to SEQ ID NO: 18 are also provided.

The skilled artisan would readily recognize that an aGzmB antibody fragment according to the present disclosure can comprise, consist essentially of, or consist of any VH domain and comprise, consist essentially of, or consist of any VL domain described herein.

In each case, where a specific amino acid sequence is recited, embodiments comprising a sequence having at least 75% to 85 to 90% or greater (e.g. 90%, 91%, 92%, 93%, 94%, 95%, 96%, 97%, 98%, or 99%) identity to the recited sequence (e.g., SEQ ID NOs: 1 or 6) are also provided.

In each case, where a specific amino acid sequence is recited, embodiments consisting essentially of a sequence having at least 75% to 85 to 90% or greater (e.g. 90%, 91%, 92%, 93%, 94%, 95%, 96%, 97%, 98%, or 99%) identity to the recited sequence (e.g., SEQ ID NOs: 1 or 6) are also provided.

In each case, where a specific amino acid sequence is recited, embodiments consisting of a sequence having at least 75% to 85 to 90% or greater (e.g. 90%, 91%, 92%, 93%, 94%, 95%, 96%, 97%, 98%, or 99%) identity to the recited sequence (e.g., SEQ ID NOS: 1 or 6) are also provided.

The disclosure also provides an antibody or antigen binding portion thereof that specifically binds to a GzmB, wherein the antibody or antigen binding portion thereof comprises an scFv comprising an amino acid sequence that is at least 95% identical (for example, at least 95%, 96%, 97%, 98% or 99% identical) to any of SEQ ID NOs: 1 or 6.

The disclosure also provides an antibody or antigen binding portion thereof that specifically binds to a GzmB, wherein the antibody or antigen binding portion thereof consists essentially of an scFv comprising an amino acid sequence that is at least 95% identical (for example, at least 95%, 96%, 97%, 98% or 99% identical) to any of SEQ ID NOs: 1 or 6.

The disclosure also provides an antibody or antigen binding portion thereof that specifically binds to a GzmB, wherein the antibody or antigen binding portion thereof consists of an scFv comprising an amino acid sequence that is at least 95% identical (for example, at least 95%, 96%, 97%, 98% or 99% identical) to any of SEQ ID NOs: 1 or 6.

In embodiments of anti-GzmB scFVs, they may also comprise any one or more N- or C-terminal tags, for example, any one of more of SEQ ID NOs: 2-5 and 7 (or tags at least 95%, 96%, 97%, 98% or 99% identical to any one of more of SEQ ID NOs: 2-5 and 7).

In embodiments of anti-GzmB scFVs, they may also comprise any one or more N-tags, for example, any one of more of SEQ ID NOs: 4 or 5 (or tags at least 95%, 96%, 97%, 98% or 99% identical to any one of more of SEQ ID NOs: 4 and 5).

In embodiments of anti-GzmB scFVs, they may also comprise any one or more C-tags, for example, any one of more of SEQ ID NOs: 2-3 or 7 (or tags at least 95%, 96%, 97%, 98% or 99% identical to any one of more of SEQ ID NOs: 2-3 or 7).

anti-GzmB scFVs can comprise, consist essentially of (i.e., retain GzmB specific binding), or consist of SEQ ID NOs. 1 or 6.

The amino acid residue sequences provided herein are set forth in single-letter amino acid code which can be used interchangeably with three-letter amino acid code. An amino acid refers to any monomer unit that can be incorporated into a peptide, polypeptide, or protein. The twenty natural or genetically encoded alpha-amino acids are as follows: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). The structures of these twenty natural amino acids are shown in, e.g., Stryer et al., Biochemistry, 5th ed., Freeman and Company (2002). The term amino acid also includes unnatural amino acids, modified amino acids (e.g., having modified side chains and/or backbones), and amino acid analogs.

As with all peptides, polypeptides, and proteins, including fragments thereof, it is understood that additional modifications in the amino acid sequence of the GzmB antigen-specific antibodies or antigen binding fragments thereof described herein, for example, in the heavy chain variable region and/or light chain variable region, can occur that do not alter the nature or function of the antibodies or antigen binding fragments thereof. Such modifications include conservative amino acids substitutions, such that each recited sequence optionally contains one or more conservative amino acid substitutions. The list provided below identifies examples of groups that contain amino acids that are conservative substitutions for one another; these groups are exemplary as other conservative substitutions are known to those of skill in the art.
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)

By way of example, when an aspartic acid at a specific residue is mentioned, also contemplated is a conservative substitution at the residue, for example, glutamic acid. Non-conservative substitutions, for example, substituting a proline with glycine, are also contemplated.

In some instances, the affinity of GzmB complex antigen-specific antibodies or antigen binding fragments thereof may be optimized through mutations to increase or decrease affinity as desired based on one or more of the known characteristics of the binding interaction with the cognant GzmB antigen. In some instances, the mutations permit facile elution of purified antibodies or fragments thereof under desirable elution conditions during isolation and purification.

Methods of generating and screening for antibodies and antigen binding fragments thereof as provided in this disclosure are described in the Examples and are well-known in the art.

The present disclosure also encompasses antibodies or fragments thereof that bind to the same epitope of GzmB antigens as the antibodies disclosed herein. Such antibodies can be identified using routine techniques known in the art, including, for example, competitive binding assays.

The present disclosure also provides chimeric antibodies. The term chimeric antibody refers to an antibody in which a component of the heavy and/or light chain is derived from a particular source or species, while the remainder of the heavy and/or light chain is derived from a different source or species.

A human antibody is one that possesses an amino acid sequence corresponding to that of an antibody produced by a human or a human cell, or derived from a non-human source that utilizes a human antibody repertoire or human antibody-encoding sequences (e.g., obtained from human sources, genetically modified non-human sources or designed de novo). Human antibodies specifically exclude humanized antibodies.

In some embodiments, the antibody or antigen binding fragment thereof provided herein can include a heavy (H) chain variable domain sequence (abbreviated herein as VH), and a light (L) chain variable domain sequence (abbreviated herein as VL). In some embodiments, an antibody molecule comprises or consists of a heavy chain and a light chain (referred to as a half antibody). In another example, an antibody molecule includes two heavy (H) chain variable domain sequences and two light (L) chain variable domain sequence, thereby forming two antigen binding sites, such as Fab, Fab′, F(ab′)₂, Fc, Fd, Fd′, Fv, single chain antibodies (scFv, for example), single variable domain antibodies, diabodies (Dab) (bivalent and bispecific), and chimeric (e.g., humanized) antibodies, which may be produced by the modification of whole antibodies or synthesized de novo using recombinant DNA technologies. These functional antibody fragments retain the ability to selectively bind with their respective antigen. Antibodies and antibody fragments can be from any class of antibodies including, but not limited to, IgG, IgA, IgM, IgD, and IgE, and from any subclass (e.g., IgG1, IgG2, IgG3, and IgG4) of antibodies. The preparation of antibody molecules can be monoclonal or polyclonal. An antibody molecule can also be a human, humanized, CDR-grafted, or an in vitro generated antibody. The antibody can have a heavy chain constant region chosen from, e.g., IgG1, IgG2, IgG3, or IgG4. The antibody can also have a light chain chosen from either kappa or lambda light chains. In embodiments, only a GzmB antibody fragment is utilized (i.e., an scFv) and not an entire antibody.

In some embodiments, the antibody or antigen binding fragment thereof are monoclonal. As used herein, the term monoclonal antibody refers to an antibody from a population of substantially homogeneous antibodies. A population of substantially homogeneous antibodies comprises antibodies that are the same or substantially similar and that bind the same epitope(s), except for variants that can normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of yeast clones, phage clones, bacterial clones, mammalian cell clones, hybridoma clones, or other recombinant DNA clones. The selected antibody can be further altered, for example, to improve affinity for the target, for example, by affinity maturation, to humanize the antibody, to improve its production in cell culture, and/or to reduce its immunogenicity in a subject.

Antigen binding fragments of an antibody molecule are well known in the art, and include, for example, (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv) (see e.g., Bird et al. (1988) Science 242:423-426; Huston et al. (1988) Proc. Natl. Acad. Sci. USA 85:5879-5883); (viii) a single domain antibody. These antibody fragments are obtained using conventional techniques known to those skilled in the art, and the fragments are screened for utility in the same manner as are intact antibodies.

Antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any of the art, or any future single domain antibodies. Single domain antibodies may be derived from any species including, but not limited to mouse, rat, guinea, pig, human, camel, llama, fish, shark, goat, rabbit, and bovine. Single domain antibodies are described, for example, in International Application Publication No. WO 94/04678. For clarity reasons, this variable domain derived from a heavy chain antibody naturally devoid of light chain is known herein as a VHH or nanobody to distinguish it from the conventional VH of four chain immunoglobulins. Such a VHH molecule can be derived from antibodies raised in Camelidae species (e.g., camel, llama, dromedary, alpaca and guanaco) or other species besides Camelidae.

In certain embodiments, antibodies and antibody compositions as provided herein are distinguishable from naturally occurring antibodies and compositions in one or more respects. Such distinguishable antibodies and compositions may be referred to as “synthetic,” or may be identified by the proviso that the antibody or composition “is not naturally occurring” or affirmatively as “non-naturally occurring.” Possible differences for synthetic antibodies include variable region sequences that differ corresponding naturally occurring antibodies, different light chain sequences (i.e. lambda type instead of kappa type or vice versa), different isotypes, different allotypes, and different constant domain variants. These differences are discussed in more detail below. In some embodiments, the synthetic antibody is an engineered polypeptide, also referred to as a recombinant polypeptide, that is made using conventional protein and antibody engineering molecular biology, chemical, and biochemical methods as described below, including, but not limited to, those described in the Examples below.

In some embodiments, the antibody comprises a heavy chain variable region sequence and a light chain variable region sequence that are derived from an immunoglobulin producing human B cell, and further comprises a kappa or lambda light chain constant region. In some embodiments, the light chain constant region (kappa or lambda) is from the same type of light chain (i.e., kappa or lambda) as the light chain variable region that was derived from the immunoglobulin producing human B cell; as a non-limiting example, if an IgE-producing human B cell comprises a kappa light chain, then the antibody that is produced can comprise the light chain variable region from the IgE-producing B cell and further comprises a kappa light chain constant region.

In some embodiments, the antibody comprises a heavy chain variable region sequence and a light chain variable region sequence that are derived from an immunoglobulin-producing human B cell, and further comprises a heavy chain constant region having an IgG isotype (e.g., IgG4), an IgA isotype (e.g., IgA1), an IgM isotype, an IgD isotype, or that is derived from an IgG, IgA, IgM, or IgD isotype (e.g., is a modified IgG4 constant region). It will be appreciated by a person of ordinary skill in the art that the different heavy chain isotypes (IgA, IgD, IgE, IgG, and IgM) have different effector functions that are mediated by the heavy chain constant region, and that for certain uses it may be desirable to have an antibody that has the effector function of a particular isotype (e.g., IgG).

In some embodiments, the antibody comprises a native (i.e., wild-type) human IgG, IgA, IgM, or IgD constant region. In some embodiments, the antibody comprises a native human IgG1 constant region, a native human IgG2 constant region, a native human IgG3 constant region, a native human IgG4 constant region, a native human IgA1 constant region, a native human IgA2 constant region, a native human IgM constant region, or a native human IgD constant region. In some embodiments, the antibody comprises a heavy chain constant region that comprises one or more modifications. It will be appreciated by a person of ordinary skill in the art that modifications such as amino acid substitutions can be made at one or more residues within the heavy chain constant region that modulate effector function. In some embodiments, the modification reduces effector function, e.g., results in a reduced ability to induce certain biological functions upon binding to an Fc receptor expressed on an effector cell that mediates the effector function. In some embodiments, the modification (e.g., amino acid substitution) prevents in vivo Fab arm exchange, which can introduce undesirable effects and reduce the therapeutic efficacy of the antibody. See, e.g., Silva et al., J Biol Chem, 2015, 280:5462-5469

In some embodiments, the antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one or more modifications that modulate effector function. Ini some embodiments the antibody comprises a human IgM constant region, human IgD constant region, human IgG constant region that is derived from IgG1, IgG2, IgG3, or IgG4, or human IgA constant region that is derived from IgA1 or IgA2. In some embodiments, the antibody comprises a native (i.e., wild-type) human IgM constant region, human IgD constant region, human IgG constant region that is derived from IgG1, IgG2, IgG3, or IgG4, or human IgA constant region that is derived from IgA1 or IgA2 and comprises one, two, three, four, five, six, seven, eight, nine, ten or more modifications (e.g., amino acid substitutions). In some embodiments the constant regions include variations (e.g., one, two, three, four, five, six, seven, eight, nine, ten or more amino acid substitutions) that reduce effector function. In some embodiments, antibodies and antibody fragments of the present disclosure can be a human IgG1 isotype-format antibody.

Synthetic antibodies and synthetic antigen-binding fragments of this disclosure may comprise variations in heavy chain constant regions to change the properties of the synthetic antibody relative to the corresponding naturally occurring antibody. Exemplary changes include mutations to modulate antibody effector function (e.g., complement-based effector function or FcγR-based effector function), alter half-like, modulate co-engagement of antigen and FcγRs, introduce or remove glycosylation motifs (glyco-engineering). See Fonseca et al., 2018, “Boosting half-life and effector functions of therapeutic antibodies by Fc-engineering: An interaction-function review” Int J Biol Macromol. 19:306-311; Wang et al., 2018, “IgG Fc engineering to modulate antibody effector functions” Protein Cell 2018, 9 (1): 63-73; Schlothauer, 2016, “Novel human IgG1 and IgG4 Fc-engineered antibodies with completely abolished immune effector functions,” Protein Engineering, Design and Selection 29 (10): 457-466; Tam et al., 2017, “Functional, Biophysical, and Structural Characterization of Human IgG1 and IgG4 Fc Variants with Ablated Immune Functionality” Antibodies 6, 12, each incorporated herein by reference for all purposes.

In some embodiments, the heavy chain variable region and/or the light chain variable region of the antibody has an identical sequence to the heavy chain variable region and/or the light chain variable region encoded by the immunoglobulin producing single B cell from the human subject having a GzmB. In some embodiments, the heavy chain variable region and/or the light chain variable region of the antibody comprises one or more modifications, e.g., amino acid substitutions, deletions, or insertions.

The heavy chain variable region sequence and/or light chain variable region sequence of an antibody or antigen binding fragment described herein can be engineered to comprise one or more variations in the heavy chain variable region sequence and/or light chain variable region sequence. In some embodiments, the engineered variation(s) improves the binding affinity of the antibody for a GzmB. In some embodiments, the engineered variation(s) improves the cross-reactivity of the antibody for a GzmB antigen.

In some embodiments, the engineered variation is a variation in one or more CDRs, e.g., an amino acid substitution in a heavy chain CDR and/or a light chain CDR as described herein. In some embodiments, the engineered variation is a variation in one or more framework regions, e.g., an amino acid substitution in a heavy chain framework region and/or a light chain framework region. In some embodiments, the engineered variation is a reversion of a region of the heavy chain and/or light chain sequence to the inferred naïve sequence. Methods for determining an inferred naïve immunoglobulin sequence are described in the art. See, e.g., Magnani et al., PLOS Negl Trop Dis, 2017, 11: e0005655, doi: 10.1371/journal.pntd.0005655

In some embodiments, affinity maturation is used to engineer further mutations that enhance the binding affinity of the antibody for a GzmB antigen or enhance the cross-reactivity of the antibody for a second GzmB antigen. Methods for performing affinity maturation are known in the art. See, e.g., Renaut et al., Methods Mol Biol, 2012, 907:451-461.

In some embodiments, an antigen binding fragment can also be or can also comprise, e.g., a non-antibody, scaffold protein. These proteins are generally obtained through combinatorial chemistry-based adaptation of preexisting antigen-binding proteins. For example, the binding site of human transferrin for human transferrin receptor can be diversified using the system described herein to create a diverse library of transferrin variants, some of which have acquired affinity for different antigens. See, e.g., Ali et al. (1999) J. Biol. Chem. 274:24066-24073. The portion of human transferrin not involved with binding the receptor remains unchanged and serves as a scaffold, like framework regions of antibodies, to present the variant binding sites. The libraries are then screened, as an antibody library is screened, and in accordance with the methods described herein, against a target antigen of interest to identify those variants having optimal selectivity and affinity for the target antigen. See, e.g., Hey et al. (2005) TRENDS Biotechnol 23 (10): 514-522.

Synthetic antibodies of this disclosure may differ from naturally occurring compositions in at least one or more of the following respects: (i) composition comprises antibodies that are purified, i.e., separated from tissue or cellular material with which they are associated in the human body, and optionally in an manufactured excipient or medium; and/or (ii) antibody compositions according to the present disclosure contain a single species of antibody (are monoclonal) such that all antibodies in the composition have the same structure and specificity. In embodiments, the antibody or antibody fragment is the GzmB scFv S17 having SEQ ID NO:1.

ii. Expression and Purification of Antibodies

The GzmB antibody fragments or antigen binding fragments thereof disclosed herein can be produced using a variety of techniques known in the art of molecular biology and protein chemistry. For example, a nucleic acid encoding the antibody or antigen binding fragment thereof can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system, such that it can be maintained in two different organisms, for example, in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells that have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed or introduced into the same cell by co-transfection (Wigler et al. (1979) Cell 16:77). A second class of vectors utilizes DNA elements that confer autonomously replicating capabilities to an extrachromosomal plasmid. These vectors can be derived from animal viruses, such as bovine papillomavirus (Sarver et al. (1982) Proc Natl Acad Sci USA, 79:7147), CMV, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO₄ precipitation, liposome fusion, cationic liposomes, electroporation, nucleoporation, viral infection, dextran-mediated transfection, polybrene-mediated transfection, protoplast fusion, and direct microinjection.

Appropriate host cells for the expression of antibodies or antigen binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13 (6): 625-629; van Kuik-Romeijn et al. (2000) Transgenic Res 9 (2): 155-159; and Pollock et al. (1999) J Immunol Methods 231 (1-2): 147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression vary with the choice of the expression vector and the host cell and are easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are known in the art (see Ausubel et al. (1988) Current Protocols in Molecular Biology, Wiley & Sons; and Green and Sambrook (2012) Molecular Cloning—A Laboratory Manual, 4th Ed., Cold Spring Harbor Laboratory Press, New York (2001)). The choice of codons, suitable expression vectors and suitable host cells vary depending on a number of factors and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

In vitro methods are also suitable for preparing monovalent antibodies, or fragments thereof. Digestion of antibodies to produce fragments thereof, particularly, Fab fragments, can be accomplished using routine techniques known in the art. For instance, digestion can be performed using papain. Examples of papain digestion are described in International Application Publication No. WO 94/29348, U.S. Pat. No. 4,342,566, and Harlow and Lane, Antibodies, A Laboratory Manual, Cold Spring Harbor Publications, New York, (1988). Papain digestion of antibodies typically produces two identical antigen binding fragments, called Fab fragments, each with a single antigen binding site, and a residual Fc fragment. Pepsin treatment yields a fragment, called the F(ab′) 2 fragment that has two antigen combining sites and is still capable of cross-linking antigen.

The Fab fragments produced in antibody digestion can also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group.

One method of producing proteins comprising the provided antibodies or fragments is to link two or more peptides or polypeptides together by protein chemistry techniques (or recombinant DNA techniques). For example, peptides or polypeptides can be chemically synthesized using currently available laboratory equipment using either Fmoc (9-fluorenylmethyl-oxycarbonyl) or Boc (tert-butyloxycarbonoyl) chemistry (Applied Biosystems, Inc.; Foster City, CA). Those of skill in the art readily appreciate that a peptide or polypeptide corresponding to the antibody provided herein, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group that is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer Verlag Inc., NY). Alternatively, the peptide or polypeptide can by independently synthesized in vivo. Once isolated, these independent peptides or polypeptides may be linked to form an antibody or fragment thereof via similar peptide condensation reactions.

For example, enzymatic ligation of cloned or synthetic peptide segments can allow relatively short peptide fragments to be joined to produce larger peptide fragments, polypeptides, or whole protein domains (Abrahmsen et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al., Science, 266:776 779 (1994)). The first step is the chemoselective reaction of an unprotected synthetic peptide a thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Baggiolini et al., FEBS Lett. 307:97-101 (1992); Clark et al., J. Biol. Chem. 269:16075 (1994); Clark et al., Biochemistry 30:3128 (1991); Rajarathnam et al., Biochemistry 33:6623-30 (1994)).

Alternatively, unprotected peptide segments can be chemically linked where the bond formed between the peptide segments as a result of the chemical ligation is an unnatural (non-peptide) bond (Schnolzer et al., Science 256:221 (1992)). This technique has been used to synthesize analogs of protein domains as well as large amounts of relatively pure proteins with full biological activity (deLisle et al., Techniques in Protein Chemistry IV. Academic Press, New York, pp. 257-267 (1992)).

Recombinant techniques can also be used to modify antibodies or antigen binding fragments thereof. For example, amino acids found to not contribute to either the activity or the binding specificity or affinity of the antibody can be deleted without a loss in the respective activity. Insertions, deletions, substitutions, or other selected modifications of particular regions or specific amino acids residues can also be made (and are contemplated by the present disclosure), provided the activity of the fragment is not significantly altered or impaired compared to the non-modified antibody, or antigen binding fragment thereof can be made. Such methods are readily apparent to a skilled practitioner in the art and can include site specific mutagenesis of the nucleic acid encoding the antibody or fragment thereof. (Zoller et al., Nucl. Acids Res. 10:6487-500 (1982)).

Following expression, the antibodies and fragments thereof can be isolated. An antibody or fragment thereof can be isolated or purified in a variety of ways known in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) Protein Purification, 3^{rd} edition, Springer-Verlag, New York City, New York. The degree of purification necessary varies depending on the desired use. In some instances, no purification of the expressed antibody or fragments thereof is necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### B. Fluorochromes

Any antibody fragment described in section (1)(A) above can be conjugated to a triple-Glycine (G₃) tagged fluorochrome at the N-terminus of the leader Glycine.

In embodiments, the fluorochrome can be a constitutively fluorescing fluorochrome or a caged fluorochrome (i.e., one that requires a stimulus to induce fluorescence).

In embodiments, the fluorochrome is a near-infrared (NIR) cyanine dye, a hemicyanine dye, a coumarin-based dye, a fluorescein-based dye, a rhodamine-based dye, a BODIPY-Based Dye, a BODIPY-Based NIR Dye, a Xanthene-Based dye, a Xanthene-Based NIR Dye, a cyanine-based dyes, a porphyrin dye, a phthalocyanine dye, a squaraine dye.

In embodiments, the fluorochrome comprises Cy5.5, Cy7, IRDye800CW, a Silicon Rhodamine, a Janelia Fluor 646, a Janelia Fluor 669, a Janelia Fluor 774); Squaraine Dyes, ICG, or IR-800.

In embodiments, the caged fluorochrome is a NIR caged cyanine or hemicyanine dye. In embodiments, the caged fluorochrome comprises CyP, a Cy dye with an IEPD peptide (i.e., Ile-Glu-Pro-Asp, SEQ ID NO:8) that is a substrate for GzmB, the stimulus that releases the “cage” and shifts the fluorescence.

In an embodiment, the G₃-tagged fluorochrome (GGG-Cy7) has the following structure (N′=>C′):

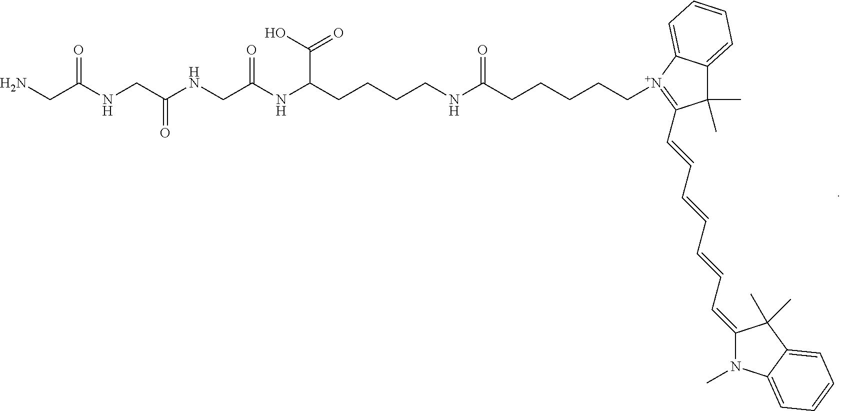 [SMILES: CN1/C(=C/C=C/C=C/C=C/C2=[N+](CCCCCC(=O)NCCCCC(NC(=O)CNC(=O)CNC(=O)CN)C(=O)O)c3ccccc3C2(C)C)C(C)(C)c2ccccc21]

In an embodiment, the caged fluorochrome (GGG-CyP) has the following structure (N′=>C′):

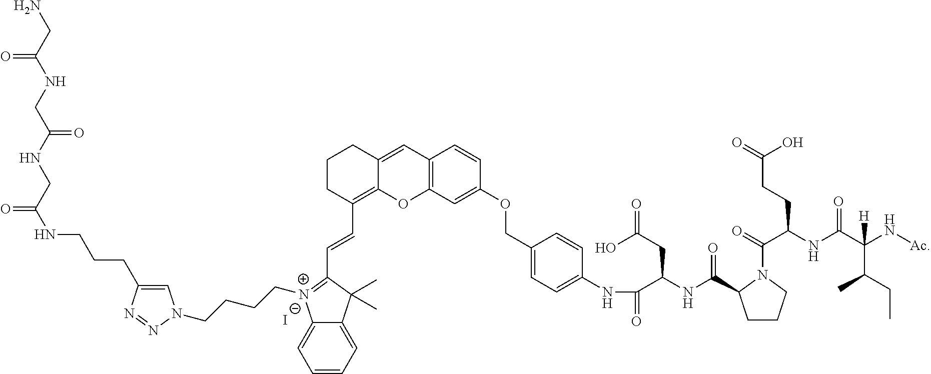

### C. Radioisotopes

Probes as described herein can also utilize radioisotopes for positron-emission tomography (PET), for example.

In embodiments, the radioisotope comprises Zirconium-89 (⁸⁹Zr), Copper-64 (⁶⁴Cu) and Fluorine-18 (¹⁸F), Technetium-99m, Iodine-131, Gallium-67, or Radium-223.

In embodiments, the radioisotope comprises ⁸⁹Zr chelated with desferrioxamine (DFO) or GGG-DFO.

Embodiments of radioisotope-tagged probes can be found, for example, in Example 2 below.

### D. Synthesis of GzmB Probes

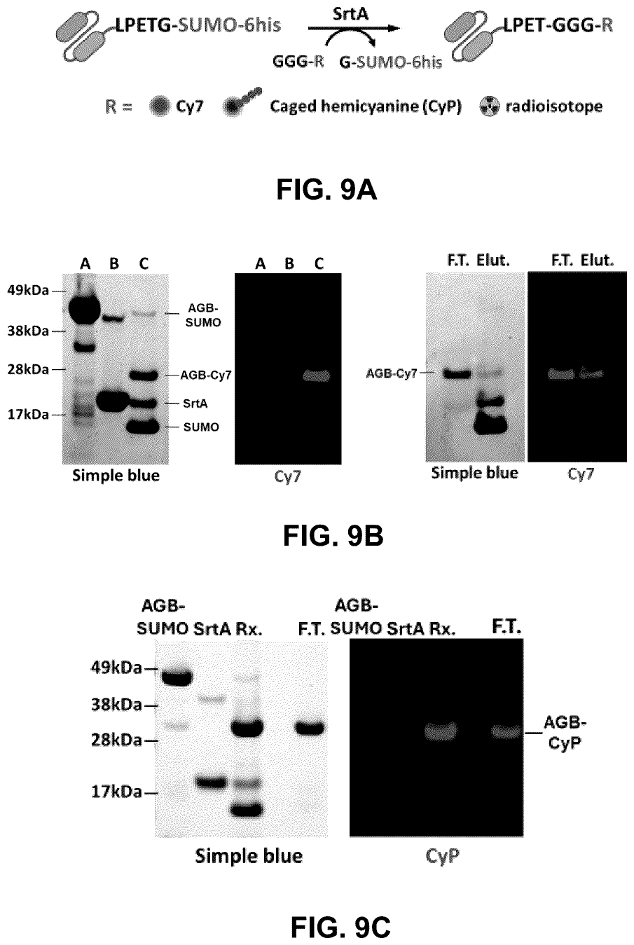

Synthesis of probes according to the present disclosure can be carried out, for example, by utilizing the synthetic routes described further in detail in the Figures and Examples below. In particular, FIG. 9A shows an embodiments of Sortase A-mediated bioligation that can be utilized to covalently conjugate anti-GzmB antibody fragments to probes as described herein.

### II. Kits and Packaging

The Granzyme B (GzmB)-targeting probes and/or compositions (i.e., those comprising, consisting essentially of, or consisting of one or more Granzyme B (GzmB)-targeting probes described herein) can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the method.

In some instances, one of the containers may comprise a composition as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label.

A composition as described in this disclosure for use in determining GzmB activity in a sample from a subject or cells suitable for TIL therapy in a sample from a subject as described herein may be delivered in a pharmaceutical package or kit to doctors and cancer patients. Such packaging is intended to improve patient convenience and compliance with the treatment plan. Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of a composition or components of compositions described herein. In some embodiments, the pharmaceutical package or kit further comprises unit dose forms of one or more of an additional therapeutic, for example, another medicament used for treatment of a disorder in a patient. The dose unit form can be a diagnostic unit dose form.

In one embodiment, the kit or pharmaceutical package comprises a composition as described herein in a defined, therapeutically effective dose in a single unit dosage form or as separate unit doses. The dose and form of the unit dose (e.g., lyophilized, reconstituted, aqueous, tablet, capsule, immediate release, delayed release, etc.) can be any doses or forms as described herein.

In certain embodiments, kits are provided for producing a single-dose administration unit, in particular for in vivo administration. In certain embodiments, kits containing single or multi-chambered pre-filled syringes are included. In certain embodiments, kits containing one or more containers of a formulation described in this disclosure are included.

### III. Methods of Use and Treatment

As described herein, in vitro and vivo studies have established degranulation and GzmB release as reliable biomarkers of CTL function. Notably, GzmB exhibits intrinsic stability and retains ~30% catalytic activity even in highly acidic cytotoxic granules before releasing. In senescent CTLs, GzmB expression is significantly downregulated. CTL exhaustion follows a hierarchical differentiation trajectory from progenitor exhausted to terminally exhausted states. Progenitor exhausted CTLs have minimal GzmB expression and no more killing effect than naïve CD8+ T cells, but retain polyfunctionality and persist long term. Terminally exhausted CTLs, by contrast, express higher levels of GzmB but are thought to have defective degranulation, leading to impaired cytotoxic function. Existing GzmB-targeted imaging probes, including small-molecule cell-permeable agents developed by the present inventors and others, nanoparticles, and cell-membrane interaction probes, fail to distinguish terminal exhaustion from functional CTLs because they detect GzmB within both T cells and engaged cancer cells.

Additionally, as described herein, existing methods for detecting functional TILs have significant drawbacks. Histological staining (IHC, IF) provides spatial localization of TILs but is inherently destructive. Flow cytometry allows multiparametric functional analysis but requires prior tissue dissociation. The high fluidic pressure can often stress T cells, reducing their viability and proliferation capacity. PCR-based techniques detect T cell-related gene expression but do not assess actual function. Moreover, these methods analyze only small aliquots of tumor resections, extrapolating findings that may not be representative due to tumor heterogeneity. There is an urgent need for a non-destructive, high-throughput method capable of screening multiple tumor fragments simultaneously, identifying functional TILs without disrupting their viability.

In embodiments, probes according to the present disclosure can be utilized in methods of screening for CTL function and functional TILs for use in TIL therapy.

In embodiments, a method of using a Granzyme B (GzmB)-targeting imaging probe is described, comprising contacting a sample with an anti-GzmB probe as described herein (or composition comprising such as described herein); incubating the contacted sample at a temperature for a period of time; and imaging the incubated sample to determine a probe level.

In embodiments, sample is a tumor resection from a tumor of a subject or a tumor or suspected tumor in a subject.

In embodiments, the tumor is a metastatic melanoma, a sarcoma, a pancreatic tumor, an ovarian tumor, a colorectal tumor, a non-small cell lung cancer (NSCLC), and a triple-negative breast cancer tumor.

In embodiments, the subject is a subject undergoing or suspected to undergo tumor-infiltrating lymphocyte (TIL) therapy or has a metastatic or unresectable melanoma that has progressed on standard immune checkpoint inhibitors and BRAF-targeted therapies, a sarcoma, a pancreatic cancer, an ovarian cancer, a colorectal cancer, a non-small cell lung cancer, or a triple-negative breast cancer. In embodiments, the sample is incubated for about one hour at about 37° C.

In embodiments, methods further comprise identifying samples for use in future TIL therapy for the subject based on a determined probe level above a threshold. In embodiments, the determined probe level is a measured fluorescence intensity. In embodiments, the identified samples can comprise the top 25-50 percentage of tumor fragments of an individual subject ranked by fluorescent readout.

The compositions described herein are useful in, inter alia, screening methods for treating a cancer using a TIL therapy and screening for functional TILs and CTL function. As used herein, the term subject means a mammalian subject. Exemplary subjects include, but are not limited to humans, monkeys, dogs, cats, mice, rats, cows, horses, camels, goats and sheep. In some embodiments, the subject is a human. In some embodiments, the subject has or is suspected to have a disorder affecting sociability. In some embodiments, the subject is diagnosed with a disorder affecting sociability. In some embodiments, the subject is a human that is suspected of having a cancer that is suitable for TIL therapy.

As used herein, administer or administration refers to the act of injecting or otherwise physically delivering a substance as it exists outside the body into a patient, such as by mucosal, intradermal, intravenous, intramuscular, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof. In particular, compositions comprising probes as described herein may be administered to a subject, for example, during a tumor resection surgery to help define the boundaries of any one or more tumors as described herein.

The compositions can be administered to a subject, e.g., a human subject, using a variety of methods that depend, in part, on the route of administration. The route can be, e.g., intravenous injection or infusion (IV), subcutaneous injection (SC), intraperitoneal (IP) injection, intramuscular injection (IM), intradermal injection (ID), subcutaneous, transdermal, intracavity, oral, intracranial injection, or intrathecal injection (IT). The injection can be in a bolus or a continuous infusion. Techniques for preparing injectate or infusate delivery systems containing antibodies are well known to those of skill in the art. Generally, such systems should utilize components which will not significantly impair the biological properties of the antibodies, such as the paratope binding capacity (see, for example, Remington's Pharmaceutical Sciences, 18th edition, 1990, Mack Publishing). Those of skill in the art can readily determine the various parameters and conditions for producing antibody injectates or infusates without resort to undue experimentation. In some embodiments, compositions as described herein can be therapeutically delivered to a subject by way of intravenous administration.

Administration can be achieved by, e.g., topical administration, local infusion, injection, and the like. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and the like.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

Other features, objects, and advantages of the present invention are apparent in the description that follows. It should be understood, however, that the description, while exemplifying certain embodiments of the present invention, is given by way of illustration only, not limitation. Various changes and modifications within the scope of the invention will become apparent to those skilled in the art from the detailed description.

### IV. Examples

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1

### A. Granzyme B as a Biomarker of Cytotoxic T Cell Function

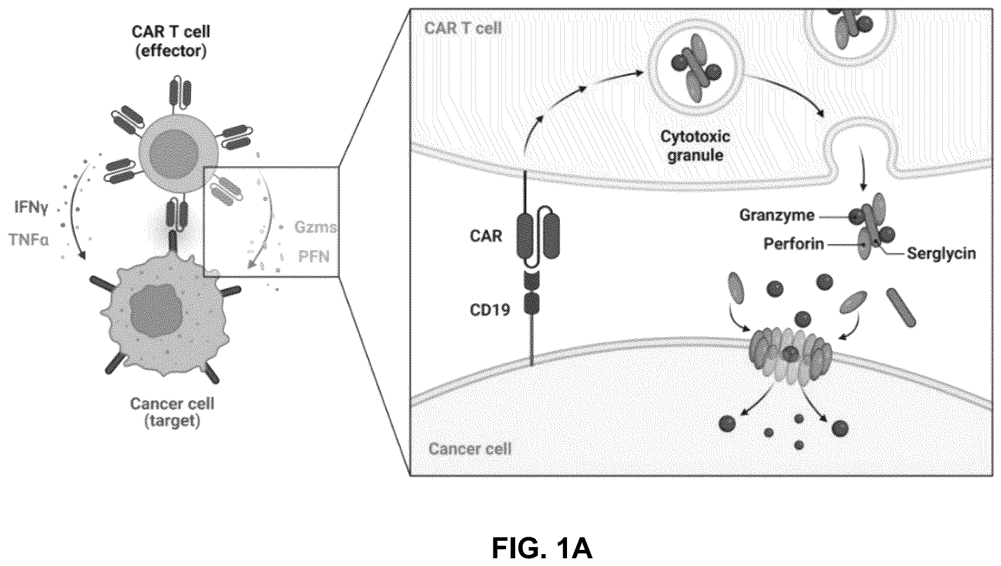

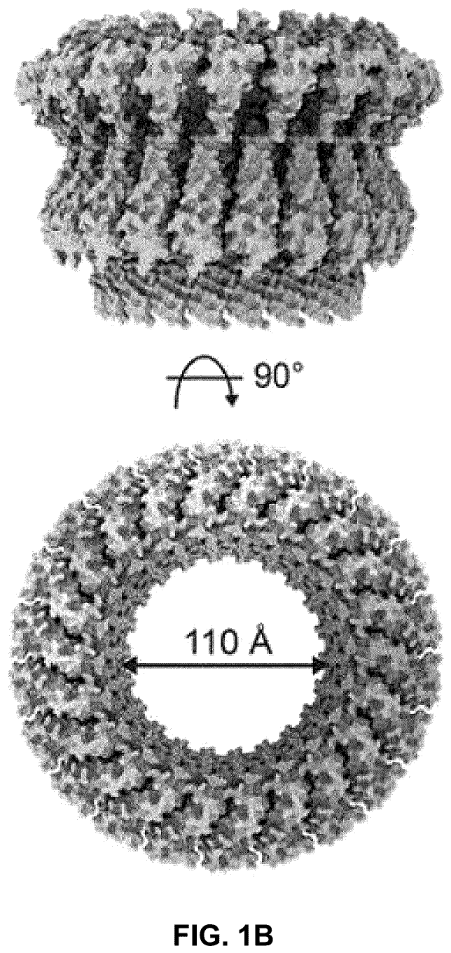

Cytotoxic T lymphocytes (CTLs) eliminate cancer cells by releasing cytotoxic granules containing perforin and granzymes, mainly granzyme B (GzmB), upon antigen activation (e.g., CAR T cells, FIG. 1A). Perforin forms pores in the cancer cell membrane. The narrowest part of these pore structures has an opening of ~110 Å in diameter (11 nm) (FIG. 1B), which is sufficient to allow passage of a GzmB monomer (~33 Kda, 2.5 nm) or a GzmA dimer (~65 Kda) into the target cell. GzmB is a potent serine proteinase (Russell J H, Ley T J. Lymphocyte-mediated cytotoxicity. Annu rev of immunol. 2002; 20:323-70. Epub 2002 Feb. 28. doi: 10.1146/annurev.immunol.20.100201.131730. PubMed PMID: 11861606), once uptake, inducing programed cell death of the cancer cell. Studies have shown that loss of GzmB reduces cytotoxic efficacy by 50-70% (Yang J, Pemberton A, Morrison W I, Connelley T. Granzyme B Is an Essential Mediator in CD8(+) T Cell Killing of Theileria parva-Infected Cells. Infect Immun. 2019; 87 (1). Epub 2018 Oct. 17. doi: 10.1128/iai.00386-18. PubMed PMID: 30323022; PMCID: PMC6300629). Beyond granule exocytosis, CTLs can mediate cell death through cytokine release or death receptor activation (Chávez-Galán L, Arenas-Del Angel M C, Zenteno E, Chavez R, Lascurain R. Cell Death Mechanisms Induced by Cytotoxic Lymphocytes. Cell Mol Immunol. 2009; 6 (1): 15-25. doi: 10.1038/cmi.2009.3.).

In vitro and vivo studies have established degranulation and GzmB release as reliable biomarkers of CTL function (Xie J, El Rami F, Zhou K, Simonetta F, Chen Z, Zheng X, Chen M, Balakrishnan P B, Dai S Y, Murty S, Alam I S, Baker J, Negrin R S, Gambhir S S, Rao J. Multiparameter Longitudinal Imaging of Immune Cell Activity in Chimeric Antigen Receptor T Cell and Checkpoint Blockade Therapies. ACS Cent Sci. 2022; 8 (5): 590-602. Epub 2022 Jun. 2. doi: 10.1021/acscentsci.2c00142. PubMed PMID: 35647285; PMCID: PMC9136971; Wherry E J, Kurachi M. Molecular and cellular insights into T cell exhaustion. Nat Rev Immunol. 2015; 15 (8): 486-99. Epub 2015 Jul. 25. doi: 10.1038/nri3862. PubMed PMID: 26205583; PMCID: 4889009). Notably, GzmB exhibits intrinsic stability (B.T. D. Generation of an expression system for human granzyme B and analysis of the in vitro and in vivo efficiency [Dissertation]. Munich: Ludwig Maximilian University of Munich; 2010) and retains ~30% catalytic activity even in highly acidic cytotoxic granules before releasing (Xie J, El Rami F, Zhou K, Simonetta F, Chen Z, Zheng X, Chen M, Balakrishnan P B, Dai S Y, Murty S, Alam I S, Baker J, Negrin R S, Gambhir S S, Rao J. Multiparameter Longitudinal Imaging of Immune Cell Activity in Chimeric Antigen Receptor T Cell and Checkpoint Blockade Therapies. ACS Cent Sci. 2022; 8 (5): 590-602. Epub 2022 Jun. 2. doi: 10.1021/acscentsci.2c00142. PubMed PMID: 35647285; PMCID: PMC9136971). In senescent CTLs, GzmB expression is significantly downregulated (Yang O O, Lin H, Dagarag M, Ng H L, Effros R B, Uittenbogaart C H. Decreased perforin and granzyme B expression in senescent HIV-1-specific cytotoxic T lymphocytes. Virol. 2005; 332 (1): 16-9. Epub 2005 Jan. 22. doi: 10.1016/j.virol.2004.11.028. PubMed PMID: 15661136). CTL exhaustion follows a hierarchical differentiation trajectory from progenitor exhausted to terminally exhausted states (Wherry E J, Kurachi M. Molecular and cellular insights into T cell exhaustion. Nat Rev Immunol. 2015; 15 (8): 486-99. Epub 2015 Jul. 25. doi: 10.1038/nri3862. PubMed PMID: 26205583; PMCID: 4889009). Progenitor exhausted CTLs have minimal GzmB expression and no more killing effect than naïve CD8+ T cells, but retain polyfunctionality and persist long term (Miller B C, Sen D R, Al Abosy R, Bi K, Virkud Y V, LaFleur M W, Yates K B, Lako A, Felt K, Naik G S, Manos M, Gjini E, Kuchroo J R, Ishizuka J J, Collier J L, Griffin G K, Maleri S, Comstock D E, Weiss S A, Brown F D, Panda A, Zimmer M D, Manguso R T, Hodi F S, Rodig S J, Sharpe A H, Haining W N. Subsets of exhausted CD8(+) T cells differentially mediate tumor control and respond to checkpoint blockade. Nat Immunol. 2019; 20 (3): 326-36. Epub 2019 Feb. 20. doi: 10.1038/s41590-019-0312-6. PubMed PMID: 30778252; PMCID: 6673650). Terminally exhausted CTLs, by contrast, express higher levels of GzmB (Miller B C, Sen D R, Al Abosy R, Bi K, Virkud Y V, LaFleur M W, Yates K B, Lako A, Felt K, Naik G S, Manos M, Gjini E, Kuchroo J R, Ishizuka J J, Collier J L, Griffin G K, Maleri S, Comstock D E, Weiss S A, Brown F D, Panda A, Zimmer M D, Manguso R T, Hodi F S, Rodig S J, Sharpe A H, Haining W N. Subsets of exhausted CD8(+) T cells differentially mediate tumor control and respond to checkpoint blockade. Nat Immunol. 2019; 20 (3): 326-36. Epub 2019 Feb. 20. doi: 10.1038/s41590-019-0312-6. PubMed PMID: 30778252; PMCID: 6673650) but are thought to have defective degranulation, leading to impaired cytotoxic function (Riches J C, Davies J K, McClanahan F, Fatah R, Iqbal S, Agrawal S, Ramsay A G, Gribben J G. T cells from CLL patients exhibit features of T-cell exhaustion but retain capacity for cytokine production. Blood. 2013; 121 (9): 1612-21. Epub 2012 Dec. 19. doi: 10.1182/blood-2012-09-457531. PubMed PMID: 23247726; PMCID: 3587324; Wu X, Zhang H, Xing Q, Cui J, Li J, Li Y, Tan Y, Wang S. PD-1 (+) CD8(+) T cells are exhausted in tumours and functional in draining lymph nodes of colorectal cancer patients. Br J Cancer. 2014; 111 (7): 1391-9. Epub 2014 Aug. 6. doi: 10.1038/bjc.2014.416. PubMed PMID: 25093496; PMCID: 4183848). Existing GzmB-targeted imaging probes, including small-molecule cell-permeable agents developed by the present inventors and others, (Xie J, El Rami F, Zhou K, Simonetta F, Chen Z, Zheng X, Chen M, Balakrishnan P B, Dai S Y, Murty S, Alam I S, Baker J, Negrin R S, Gambhir S S, Rao J. Multiparameter Longitudinal Imaging of Immune Cell Activity in Chimeric Antigen Receptor T Cell and Checkpoint Blockade Therapies. ACS Cent Sci. 2022; 8 (5): 590-602. Epub 2022 Jun. 2. doi: 10.1021/acscentsci.2c00142. PubMed PMID: 35647285; PMCID: PMC9136971; Scott J I, Gutkin S, Green O, Thompson E J, Kitamura T, Shabat D, Vendrell M. A Functional Chemiluminescent Probe for in Vivo Imaging of Natural Killer Cell Activity Against Tumours. Angew Chem Int Ed Engl. 2021; 60 (11): 5699-703. Epub 2020 Dec. 11. doi: 10.1002/anie.202011429. PubMed PMID: 33300671; PMCID: PMC7986153; Scott J I, Mendive-Tapia L, Gordon D, Barth N D, Thompson E J, Cheng Z, Taggart D, Kitamura T, Bravo-Blas A, Roberts E W, Juarez-Jimenez J, Michel J, Piet B, de Vries I J, Verdoes M, Dawson J, Carragher N O, Connor R A O, Akram A R, Frame M, Serrels A, Vendrell M. A fluorogenic probe for granzyme B enables in-biopsy evaluation and screening of response to anticancer immunotherapies. Nat Commun. 2022; 13 (1): 2366. Epub 2022 May 3. doi: 10.1038/s41467-022-29691-w. PubMed PMID: 35501326; PMCID: PMC9061857) nanoparticles (He S, Li J, Lyu Y, Huang J, Pu K. Near-Infrared Fluorescent Macromolecular Reporters for Real-Time Imaging and Urinalysis of Cancer Immunotherapy. J Am Chem Soc. 2020; 142 (15): 7075-82. Epub 2020 Mar. 21. doi: 10.1021/jacs.0c00659. PubMed PMID: 32196318; 22. Xu L, Liu N, Zhan W, Deng Y, Chen Z, Liu X, Gao G, Chen Q, Liu Z, Liang G. Granzyme B Turns Nanoparticle Fluorescence “On” for Imaging Cytotoxic T Lymphocyte Activity in Vivo. ACS Nano. 2022; 16 (11): 19328-34. Epub 2022 Oct. 26. doi: 10.1021/acsnano.2c08896. PubMed PMID: 36282211) and cell-membrane interaction probes (Zhao N, Bardine C, Lourenço A L, Wang Y H, Huang Y, Cleary S J, Wilson D M, Oh D Y, Fong L, Looney M R, Evans M J, Craik C S. In Vivo Measurement of Granzyme Proteolysis from Activated Immune Cells with PET. ACS Cent Sci. 2021; 7 (10): 1638-49. Epub 2021 Nov. 4. doi: 10.1021/acscentsci.1c00529. PubMed PMID: 34729407; PMCID: PMC8554823) fail to distinguish terminal exhaustion from functional CTLs because they detect GzmB within both T cells and engaged cancer cells.

### B. Results

### Design of AGB-CyP Fluorogenic and AGB-Cy7 Fluorescence Probes for Imaging Functional CTLs

Two imaging probes were developed, AGB-CyP and AGB-Cy7, through the re-engineering and chemical modification of a previously reported human granzyme B scFv construct, S17 (Rong J, Xu X, Ewen C, Bleackley R C, Kane K P. Isolation and characterization of novel single-chain Fv specific for human granzyme B. Hybrid Hybrid. 2004; 23 (4): 219-31. Epub 2004 Aug. 21. doi: 10.1089/1536859041651349. PubMed PMID: 15319069). S17 was originally designed for immunoprecipitation studies and exhibited nanomolar binding affinity to activated human granzyme B (Rong J, Xu X, Ewen C, Bleackley R C, Kane K P. Isolation and characterization of novel single-chain Fv specific for human granzyme B. Hybrid Hybrid. 2004; 23 (4): 219-31. Epub 2004 Aug. 21. doi: 10.1089/1536859041651349. PubMed PMID: 15319069). However, its poor solubility and limited scalability for expression and purification necessitated a redesign.

Based on S17, a new anti-GzmB antibody construct named AGB was created, and AGB-CyP and AGB-Cy7 probes were developed for optical imaging. AGB-CyP is a fluorogenic probe activated by GzmB, while AGB-Cy7 is a fluorescent probe designed for continuous emission. Both probes offer potential for imaging degranulated GzmB in extracellular environments or within CTLs engaged cancer cells.

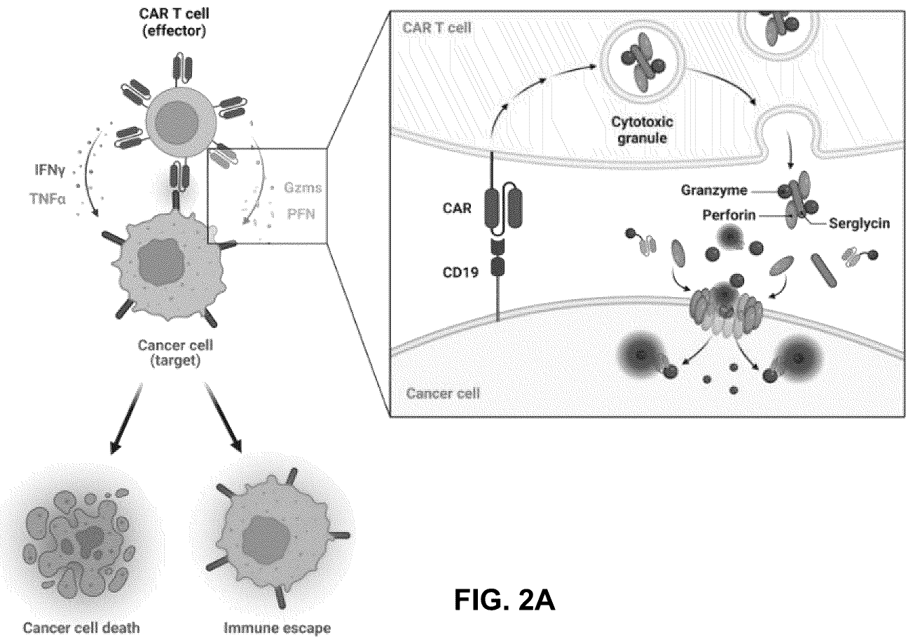

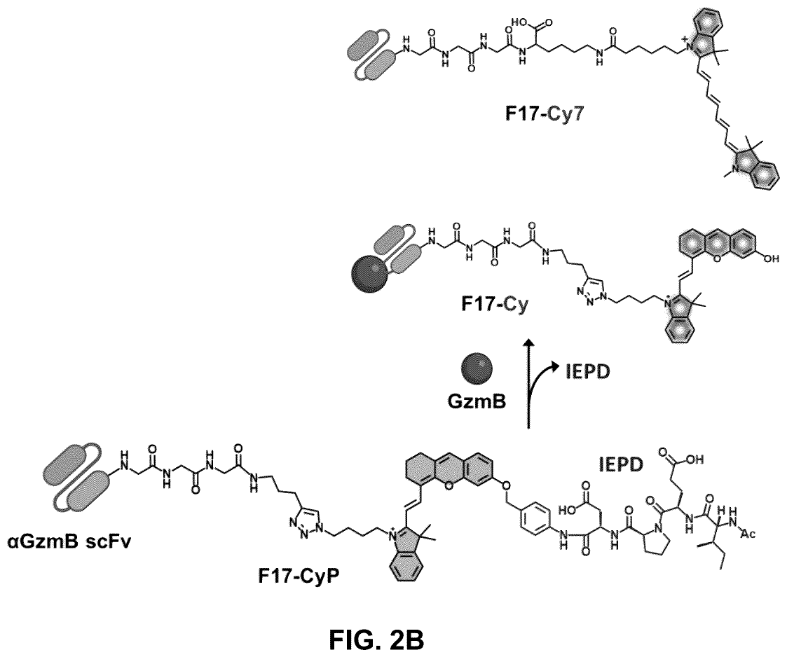

As illustrated in FIGS. 2A-2B, AGB-CyP combines the re-engineered scFv antibody at the N-terminus and an IEPD (SEQ ID NO:8) peptide-caged hemicyanine dye at the C-terminus. The hemicyanine dye exhibits high quantum yield in the near-infrared range thus ideal for tissue and in vivo imaging applications. IEPD (SEQ ID NO:8) is a substrate specific to human GzmB. AGB-CyP, with an estimated molecular weight of ~32 kDa (<2.5 nanometer), is non-cell permeable. During active degranulation, AGB-CyP binds and is activated specifically by released GzmB, influxing through perforin pores (~11 nanometer) to selectively label cancer cells undergoing cytotoxicity (FIG. 2A). This retains the fluorescence signal in situ where functional CTLs are actively killing cancer cells. Unlike existing GzmB probes, AGB-CyP (FIG. 2B) does not detect GzmB inside CTLs, allowing it to differentiate functional CTLs from terminally exhausted CTLs. AGB-Cy7, the “always-on” fluorescent probe, served as a proof-of-concept tool to validate the synthesis and optimize imaging protocols.

### Design, Expression, and Purification of AGB

The antigen-binding loops of S17 were preserved while optimizing the N- and C-terminal sequences to improve solubility, facilitate expression, and enable efficient sortase A ligation (FIGS. 3A-3B). Modifications included the addition of a pelB signal peptide for periplasmic translocation and tandem FLAG tags (S3FLAG) to lower the isoelectric point, enhancing solubility. The incorporation of SUMO and a 6×His tag simplified purification and provided a 12 kDa cleavable handle for sortase A-mediated ligation.

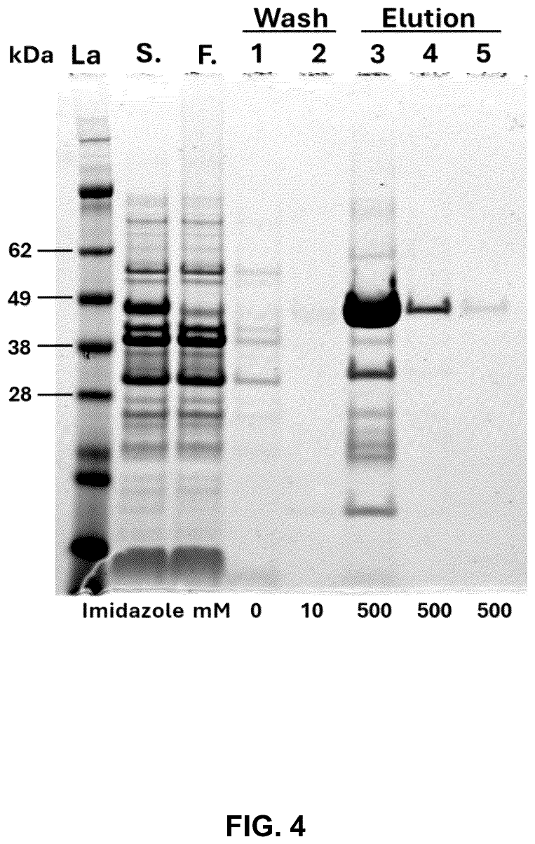

AGB was expressed in E. coli BL21 cells carrying the pET-30b (+)-AGB plasmid. After IPTG induction and cell lysis, the protein was purified from periplasm via metal-affinity chromatography using Ni-NTA resin. SDS-PAGE analysis confirmed successful expression and purification, yielding approximately 1.5 mg of AGB protein per liter of culture (FIG. 4).

### Chemical Synthesis of GGG-Cy7 and GGG-CyP

### GGG-Cy7

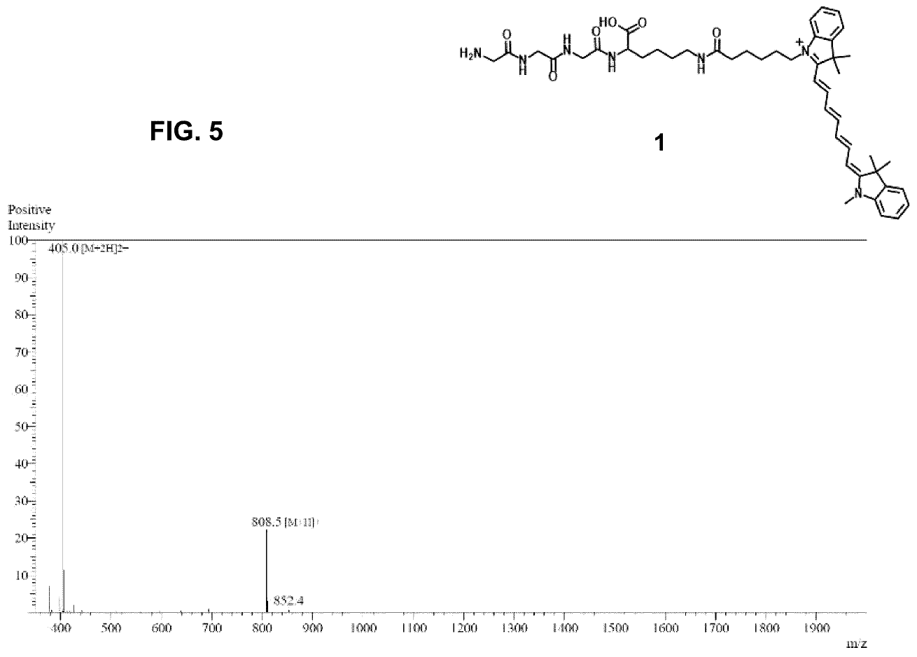

For the synthesis of GGG-Cy7 (compound 1, FIG. 5), SPPS chemical method was used for peptide production. The first Fmoc amino acid is attached to resin. Deprotection of Fmoc is accomplished by treatment of the amino acid with a base. Then coupled next Fmoc amino acids till the last Glycine, then deprotected protecting group of Lysine, then modified Cy7 dye. After the desired peptide is synthesized, the resin bound peptide is deprotected and detached from the solid support via cleavage reagents. The compound was confirmed with mass spectrometry (M.W. 808, FIG. 5).

### GGG-CyP

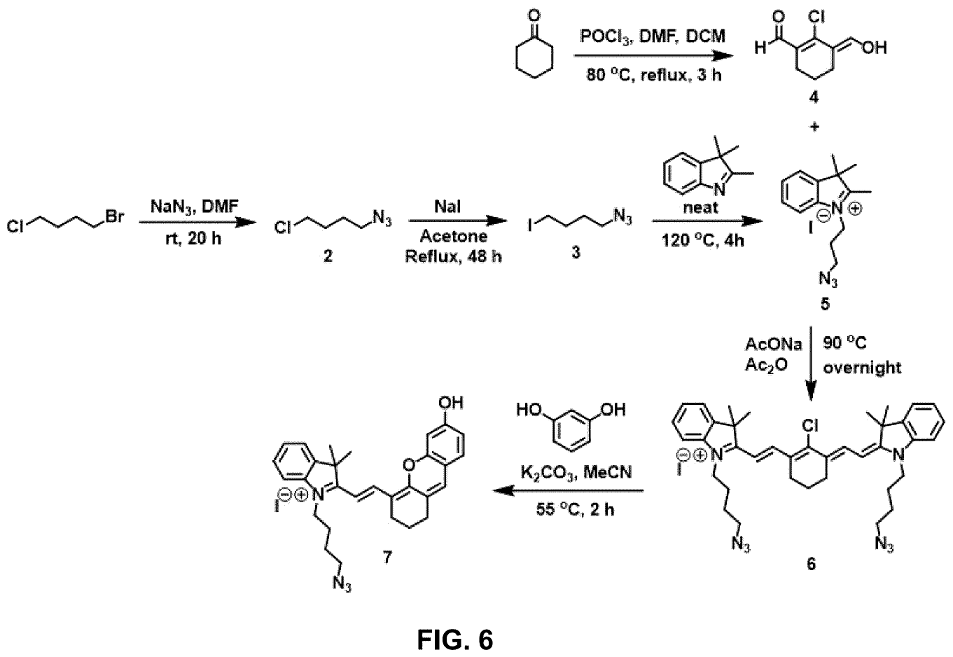

Synthesis of Compound 2 (FIG. 6): Sodium azide (1.1 g, 17 mmol) was added to a solution of 1-bromo-4-chlorobutane 1 (3.0 g, 17 mmol) in 30 mL of DMF. The reaction was stirred at room temperature for 20 h. H₂O (50 mL) was then added to the reaction mixture. The mixture was extracted three times with Et₂O (30 mL each time). The organic layers were combined and washed twice with H₂O (50 mL each) and once with brine (50 mL). The crude product was dried over Na₂SO₄, and the solvent was removed using a rotavap. The product was obtained as a clear liquid (2.22 g, 95%).

Synthesis of Compound 3: Compound 2 (2.0 g, 15 mmol) was dissolved in acetone (36 mL), and sodium iodide (4.5 g, 30 mmol) was added to the mixture. The reaction was stirred and refluxed for 48 h. After cooling to room temperature, 50 mL of H₂O was added, and the mixture was extracted three times with Et₂O (30 mL each time). The combined organic layers were dried over Na₂SO₄, and the solvent was removed using a rotavap. The product was obtained as a dark brown liquid (2.78 g, 83%).

Synthesis of Compound 4: POCl₃ (11 mL, 118 mmol) in DCM (2.5 mL) was added dropwise over 30 minutes to a solution of DMF (11 mL, 160 mmol) in DCM (10 mL) at 0° C. Cyclohexanone (2.6 mL, 25 mmol) was then added to the resulting mixture. The reaction was stirred and refluxed at 80° C. for 3 h. After cooling to room temperature, the crude product was slowly added to cold water (200 mL). The mixture was left overnight to obtain the yellow solid. The yellow solid was then filtered as compound 4 (3.11 g, 71%).

Synthesis of Compound 5: 2,3,3-Trimethylindolenine (0.30 g, 1.9 mmol) and compound 3 (0.47 g, 2.1 mmol) were mixed. The reaction mixture was heated at 120° C. for 4 h. The residue was dissolved in 1 mL of DCM, and 15 mL of cold Et₂O was added to precipitate the product. The product was washed with cold Et₂O and filtered to obtain a black solid, which was used directly for the next reaction step without further purification.

Synthesis of Compound 6: Compound 4 (0.12 g, 0.71 mmol), compound 5 (0.60 g, 1.56 mmol), and sodium acetate (0.06 g, 0.73 mmol) were dissolved in 5 ml of acetic anhydride. The reaction mixture was stirred at 90° C. for 4 h. The residue was extracted three times with saturated NaHCO₃ (mL each time) to remove excess acetic anhydride. The crude product was purified by silica gel chromatography (gradient DCM: MeOH 100:1 to 15:1) to obtain compound 6 as a green solid (0.22 g, 40%).

Synthesis of Compound 7: K₂CO₃ (0.13 g, 0.9 mmol) and resorcinol (0.1 g, 0.9 mmol) were ground and added to 5 mL of ACN at 55° C. for 15 minutes. Compound 6 (0.2 g, 0.3 mmol) in 5 mL of ACN was then added slowly to the reaction mixture. The reaction was stirred for 2 h. The residue was diluted with DCM and washed with H₂O. The crude product was dried over Na₂SO₄, and the solvent was removed using a rotavap. The residue was purified by silica gel chromatography (gradient DCM: MeOH) to obtain compound 7 as a blue solid (g, %).

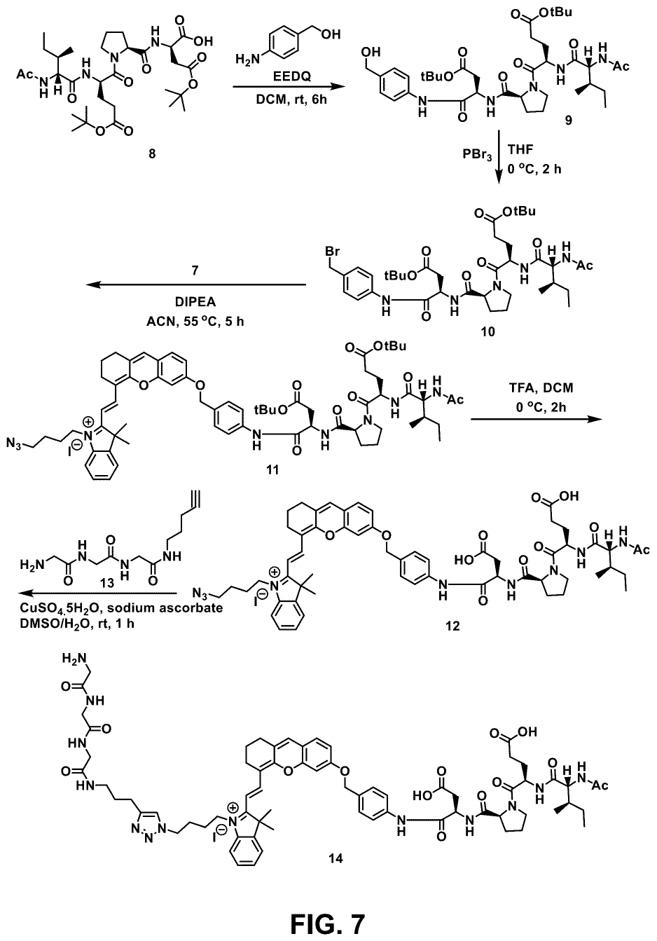

Synthesis of Compound 9 (FIG. 7): Compound 8 (Ac-Ile-Glu-Pro-Asp-OH, i.e., Ac-IEPD-OH; SEQ ID NO:9) (0.38 g, 0.6 mmol) was dissolved in DCM. N-Ethoxycarbonyl-2-ethoxy-1,2-dihydroquinoline (EEDQ, 0.59 g, 2.4 mmol) was added to the mixture, and the reaction was stirred for 20 minutes. 4-Aminobenzyl alcohol (0.29 g, 2.4 mmol) dissolved in DCM was then added via syringe, and the reaction was stirred at room temperature for 6 h. The crude product was purified by preparative HPLC to obtain compound 9 (g, %).

Synthesis of Compound 10: Compound 9 (88 mg, 0.12 mmol) was dissolved in THF in ice bath. PBr₃ (23 μL, 0.24 mmol), dissolved in THF, was added to the reaction mixture via syringe. The reaction mixture was stirred for 2 h. The solvent was then removed using a rotavap. The resulting mixture was diluted with EtOAc and washed with saturated NaHCO₃. The organic layer was dried over Na₂SO₄, and the solvent was removed using a rotavap. The crude product was used for the next step without further purification.

Synthesis of Compound 11: Compound 7 (15 mg, 0.025 mmol) and N,N-Diisopropylethylamine (DIPEA, 17.5 μL, 0.10 mmol) were dissolved in ACN and stirred at 55° C. for 20 minutes. Compound 10 (80 mg, 0.10 mmol), dissolved in ACN, was then added dropwise to the reaction mixture, and the reaction was stirred at 55° C. for 5 h. The solvent was removed using a rotavap, and the residue was purified by preparative HPLC to obtain compound 11 (g, mmol).

Synthesis of Compound 12: Compound 11 (26 mg, 0.02 mmol) was stirred in TFA/DCM (50% v/v) in an ice bath for 2 h. The reaction was monitored by HPLC. Saturated NaHCO₃ was added dropwise to the reaction mixture until the pH reached 7.4. Compound 12 was obtained (g, %) by preparative HPLC.

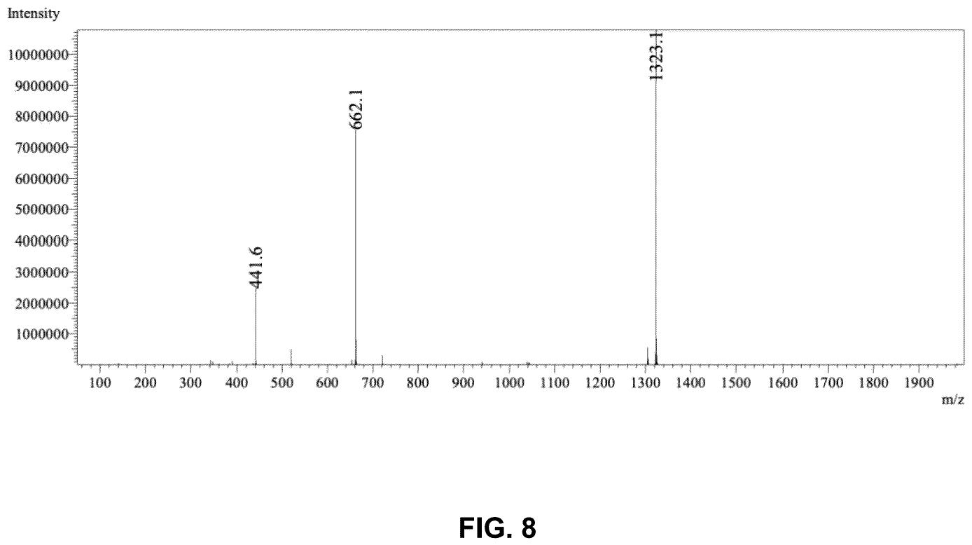

Synthesis of Compound 14: Compound 13 (3.8 mg, 0.015 mmol), compound 12 (18 mg, 0.105 mmol), CuSO₄·5H₂O (4 mg, 0.015 mmol), and sodium ascorbate (1.5 mg, 0.007 mmol) were dissolved and stirred in DMSO/H₂O (50% v/v) for 1 h. The reaction mixture was then purified by preparative HPLC to obtain compound 14 (g, %) that was confirmed by mass spectrometry (FIG. 8).

### Sortase a Mediated Bio-Ligation to Generate AGB-Cy7 and Fluorogenic AGB-CyP Probes

Sortase A (SrtA), a transpeptidase from Staphylococcus aureus, facilitated bio-ligation of fluorescent dyes and chelators to AGB (Guimaraes C P, Witte M D, Theile C S, Bozkurt G, Kundrat L, Blom A E M, Ploegh H L. Site-specific C-terminal and internal loop labeling of proteins using sortase-mediated reactions. Nat Protoc. 2013; 8 (9): 1787-99. doi: 10.1038/nprot.2013.101). SrtA recognizes the LPXTG sequence of AGB and ligates GGG-Cy7 and GGG-CyP. The labeling efficiency exceeded 95% after overnight incubation at 4° C. with an AGB: SrtA: GGG-Cy7 or GGG-CyP molar ratio of 1:0.1:20 (FIGS. 9A-9C).

### AGB-Cy7 Binds Specifically to Human GzmB

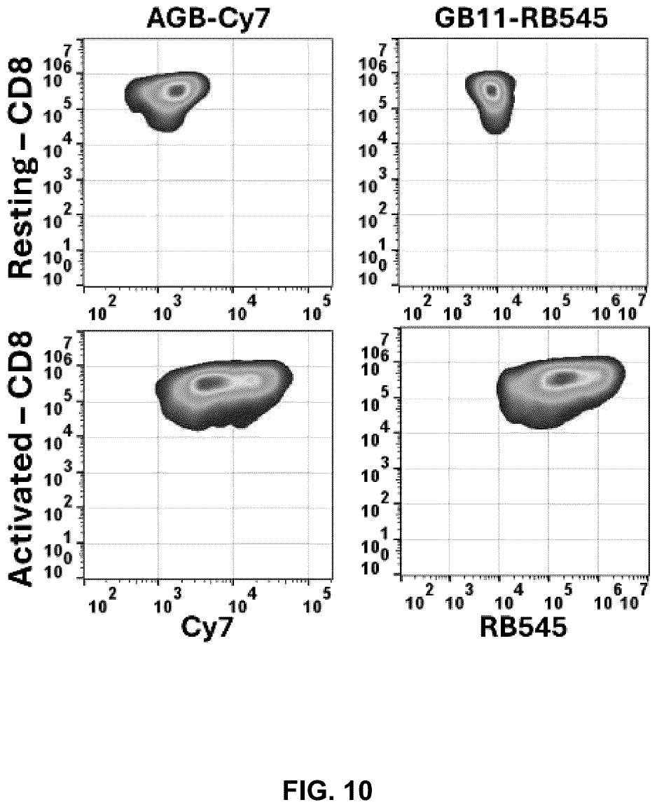

To validate the binding specificity of AGB probes to human GzmB, AGB-Cy7 was applied to stain resting and activated CD8 T cells. A commercial human GzmB monoclonal antibody conjugated with RB545 (GB11-RB545) was used as a control. As shown in FIG. 10, AGB-Cy7 demonstrates a comparable GzmB binding to the commercial monoclonal antibody (FIG. 10).

AGB-Cy7 Selectively Labels Released GzmB from CAR T Cells

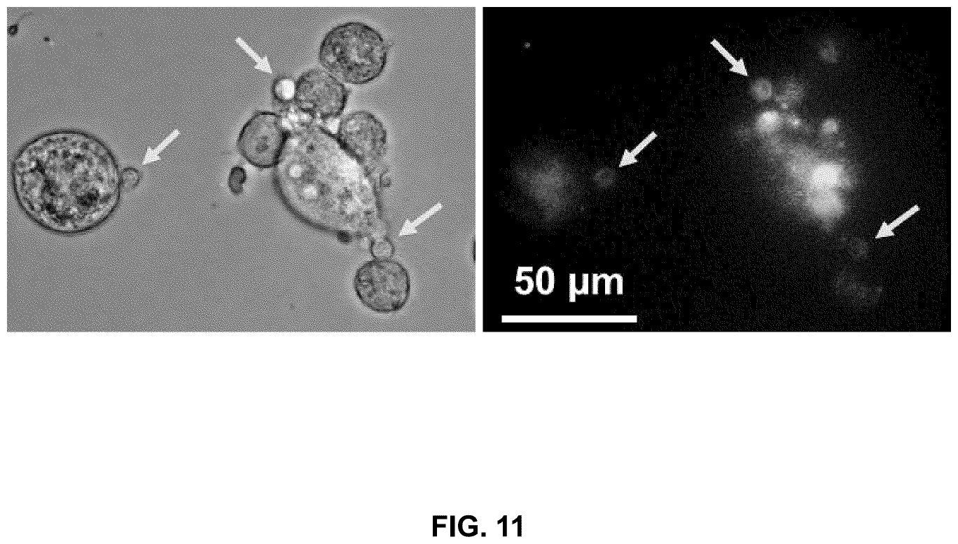

To confirm if AGB-Cy7 could label released GzmB during functional T cell mediated cytotoxicity, GFP-expressing CD19-28ζ CAR T cells were engineered from a healthy donor and incubated at 1:1 ratio overnight with glioblastoma cells (U87 MG) expressing CD19 in the presence of AGB-Cy7. As shown in FIG. 11, only apoptotic cancer cells killed by CAR T cells exhibited red fluorescence, confirming AGB-Cy7 binding to released GzmB. In comparison, no red fluorescence was observed in T cells (yellow arrows).

### AGB-Cy7 Labels Cancer Cells Escaped T Cell Cytotoxicity

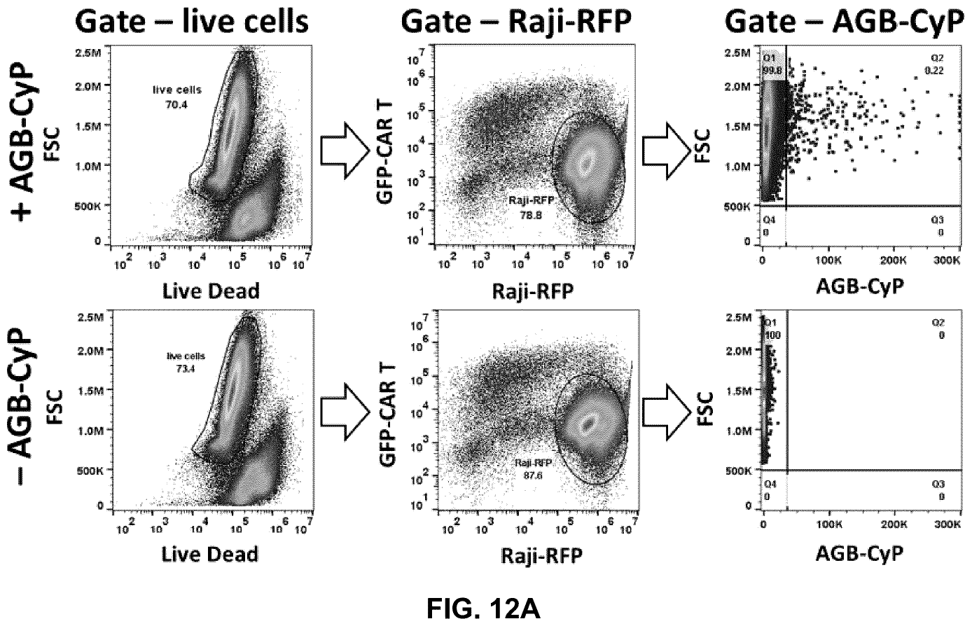

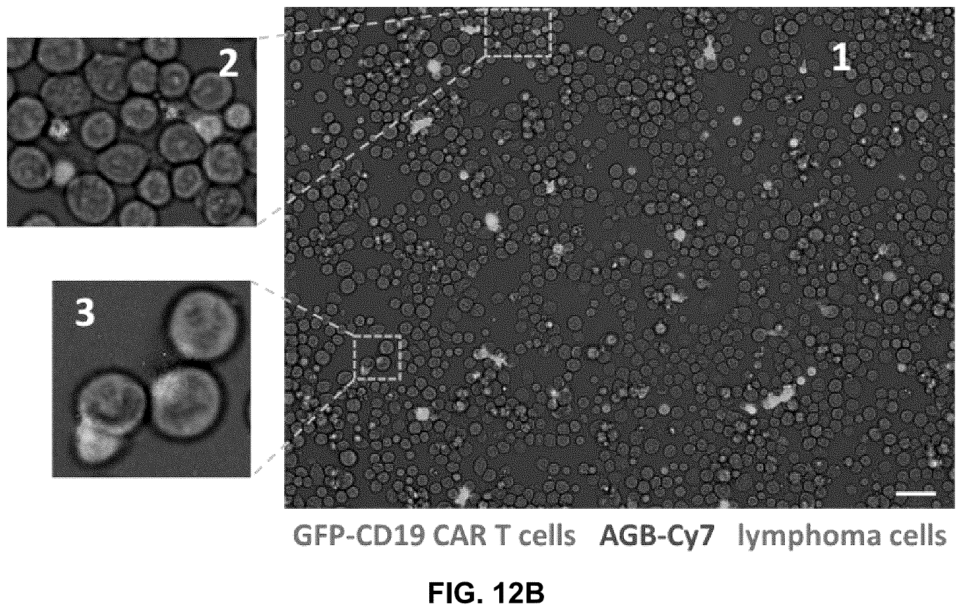

AGB-Cy7 was applied for labeling CAR T cells engaged Raji lymphoma cells in suspended culture. Flow cytometry revealed a small population (0.22%) of live, AGB-Cy7-positive Raji cells, indicating prior engagement with CAR T cells but escape cytotoxic killing (FIG. 12A). Fluorescence microscopy confirmed the uptake of AGB-Cy7 in apoptotic (FIG. 12B2) and escaped (FIG. 12B3) lymphoma cells.

### Establishment of a Lymphoma Spheroid CAR T-Cell Model

Compared to 2D cancer cell culture, 3D tumor spheroids replicate the structure and function of real tumors, making it possible to generate exhausted T cells in a human malignancy background in vitro. However, tumor spheroids are much less popular than 2D cancer cell culture due to issues with consistency, scalability, technical complexity, and cost. Traditional methods for growing tumor spheroids, such as using agarose-coated plates, inverted small droplets, or microcarrier beads, often result in variable sizes.

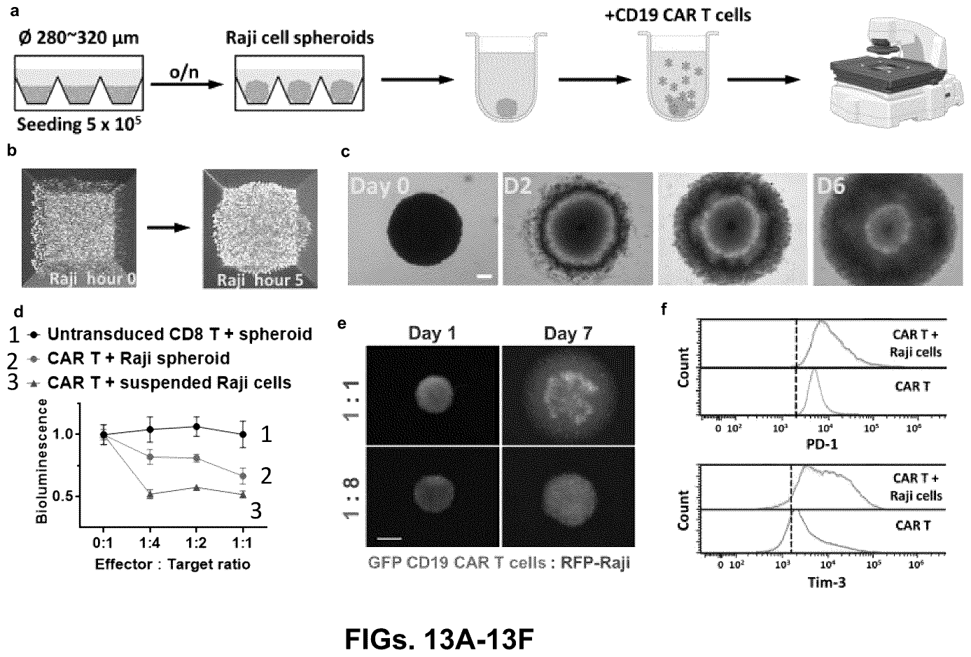

A simpler and quicker method has been developed for producing human lymphoma spheroids which provides a live tissue environment to study T cell function. As demonstrated in FIG. 13A, 5×10⁵ Raji cells were seeded in an AggreWell 800 24-well culture plate 26. Each of wells contains 300 mini square conical wells, and once centrifuged, the cancer cells pellet tightly. Within five hours, the Raji cells in each mini-well assembled into one spheroid (FIG. 13B), generating 300 homogeneous spheroids. After proper dilution and transfer, each spheroid can be further grown in a Nunc 96-well round-bottom culture plates.

The spheroids were incubated with GFP CD19 CAR T cells and efficient infiltration was observed (FIG. 13C), although their killing efficiency was compromised than suspended cancer cells (FIG. 13D). By adjusting the effector-to-target ratio, it was observed that under high tumor antigen burden (1:8), the GFP-CAR T cells could still infiltrate but lost their ability to control the spheroid growth over 7 days (FIG. 13E). The following flow cytometry analysis indicated T cell exhaustion marked by elevated PD-1 and Tim-3 expression (FIG. 13F).

### Enhanced Imaging Properties of AGB-CyP

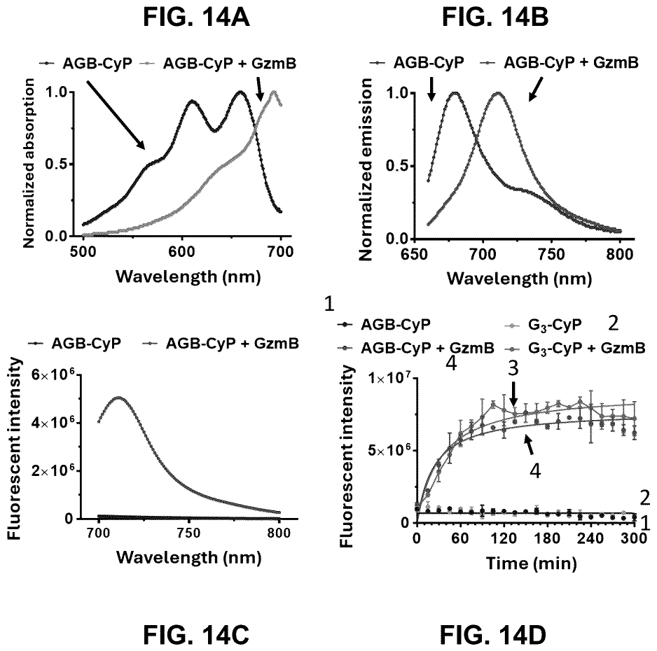

Fluorescence imaging in tissue or in vivo is often hindered by background autofluorescence, light scattering, and limited penetration depth. AGB-CyP addresses these challenges through near-infrared fluorescence activation. Before GzmB activation, AGB-CyP exhibits dual excitation peaks at 610 nm and 660 nm (FIG. 14A), with weak emission at 675 nm (FIG. 14B). Upon activation, fluorescence red shifts to near-infrared Ex690/Em715 (FIGS. 14A, 14B), yielding a ~60-fold signal increase (FIG. 14C). These properties enable high-contrast, deep-tissue imaging suitable for tumor tissues in vivo and ex vivo. Importantly, AGB-CyP binding does not interfere with GzmB's catalytic activity (FIG. 14D).

### AGB-CyP is Safe for T Cell Imaging

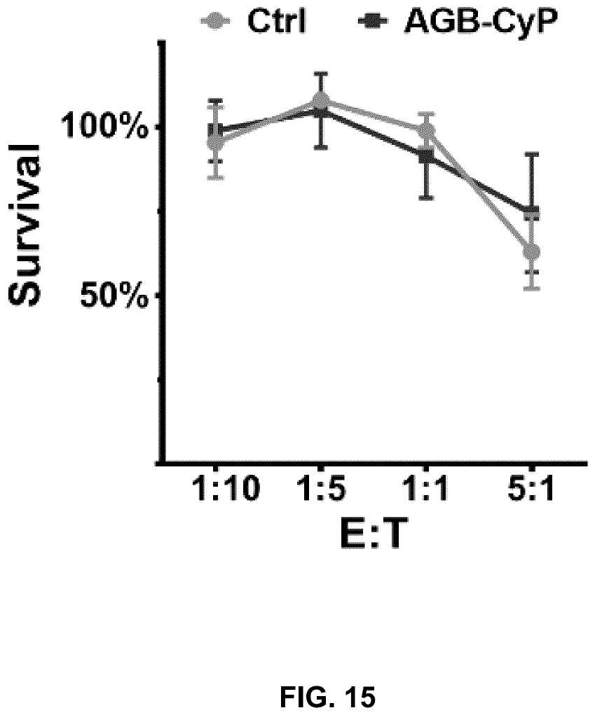

To evaluate potential cytotoxic effects of AGB-CyP on T cell function, GFP CAR T cells were incubated with luciferase-expressing lymphoma cells (Raji-Fluc) in the presence of 200 nM AGB-CyP. A bioluminescence assay after 24 hours confirmed no reduction in CAR T cell viability or cytotoxic activity (FIG. 15), demonstrating AGB-CyP's biocompatibility and suitability for in/ex vivo T cell imaging and future clinical applications.

### AGB-CyP Detects Tumor-Infiltrating Lymphocytes (TILs) in 3D Tumor Models and Tissue Phantoms Mimicking Fragmented Tumor Resections

The sensitivity of AGB-CyP in detecting infiltrating CAR T cells in 3D tumor spheroid and tissue phantom models was evaluated. Lymphoma tumor spheroids were established with 10⁴ Raji-RFP cells naturally expressing CD19. Then the spheroids were treated with 10², 10³, or 10⁴ GFP CD19-28ζ CAR T cells and the models cultured for 96 hours. AGB-CyP was added at 200 nanomolar concentration and incubated with the spheroids for one hour before gentle washing and imaging.

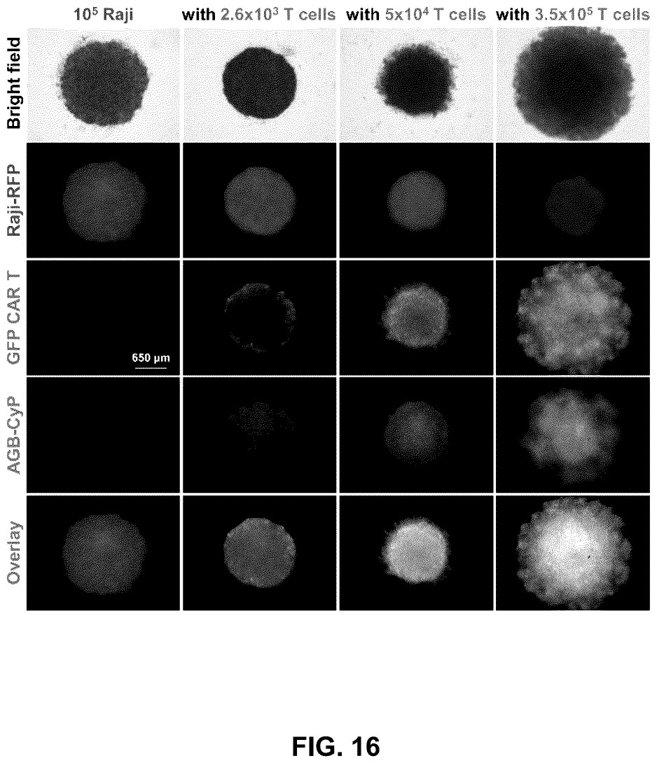

As shown in FIG. 16, CAR T cells successfully infiltrated and expanded within the tumor spheroids over 96 hours, effectively controlling tumor growth. Following spheroid dissociation and cell quantification, it was determined that AGB-CyP could reliably detect as few as 2.6×10³ infiltrated CAR T cells.

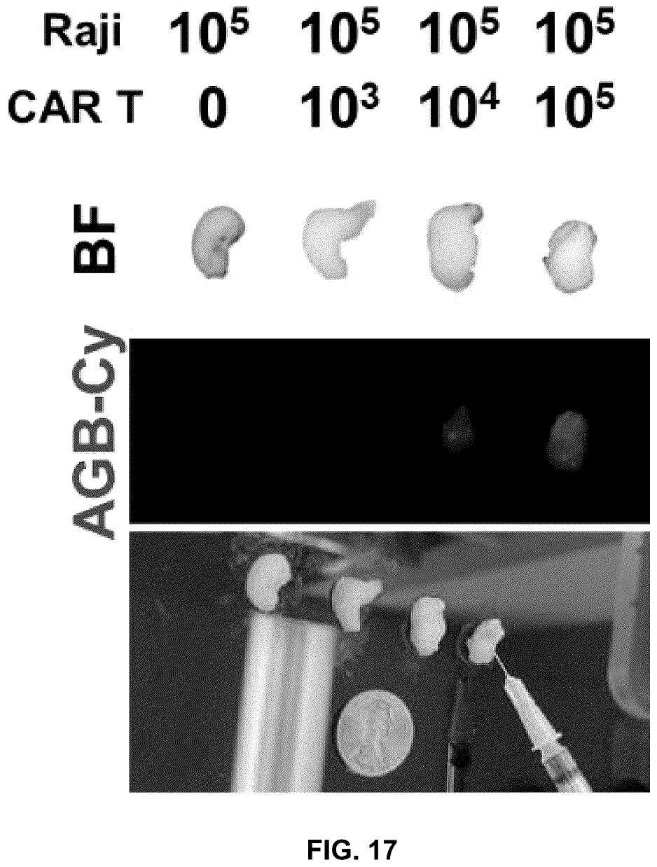

To further assess the feasibility of fluorogenic TIL imaging in tissue, CAR T cell-treated spheroids were resuspended after 96 hours in 10 microliters of PBS buffer containing 200 nanomolar AGB-CyP and they were injected into fragmented chicken breast tissue, each piece weighing approximately 0.2 to 0.3 grams. As demonstrated in FIG. 17, fluorescence scanning after a one-hour incubation revealed that as few as 10⁴ CAR T cells could be detected.

Together, these results validate the design of AGB-CyP, demonstrating its superior sensitivity over clinically estimated 10⁶ to 10⁷ TILs per gram of tumor.

### C. References Related to the Present Example

1. Khalil D N, Smith E L, Brentjens R J, Wolchok J D. The future of cancer treatment: immunomodulation, CARs and combination immunotherapy. Nat Rev Clin Oncol. 2016; 13 (5): 273-90. Epub 2016 Mar. 16. doi: 10.1038/nrclinonc.2016.25. PubMed PMID: 26977780; PMCID: 5551685.
2. Kruger S, Ilmer M, Kobold S, Cadilha B L, Endres S, Ormanns S, Schuebbe G, Renz B W, D′Haese J G, Schloesser H, Heinemann V, Subklewe M, Boeck S, Werner J, von Bergwelt-Baildon M. ACI 2019-latest trends. J Exp Clin Cancer Res. 2019; 38 (1): 268. Epub 2019 Jun. 21. doi: 10.1186/s13046-019-1266-0. PubMed PMID: 31217020; PMCID: 6585101.
3. Ando M, Ito M, Srirat T, Kondo T, Yoshimura A. Memory T cell, exhaustion, and tumor immunity. Immunol Med. 2020; 43 (1): 1-9. doi: 10.1080/25785826.2019.1698261. PubMed PMID: 31822213.
4. Cohen A D, Raje N, Fowler J A, Mezzi K, Scott E C, Dhodapkar M V. How to Train Your T Cells: Overcoming Immune Dysfunction in Multiple Myeloma. Clin Cancer Res. 2020; 26 (7): 1541-54. doi: 10.1158/1078-0432.CCR-19-2111. PubMed PMID: 31672768.
5. Lei X, Lei Y, Li J K, Du W X, Li R G, Yang J, Li J, Li F, Tan H B. Immune cells within the tumor microenvironment: Biological functions and roles in cancer immunotherapy. Cancer Lett. 2020; 470:126-33. doi: 10.1016/j.canlet.2019.11.009. PubMed PMID: 31730903.
6. Minton K. Overcoming CAR T cell exhaustion. Nat Rev Immunol. 2020; 20 (2): 72-3. doi: 10.1038/s41577-019-0265-x. PubMed PMID: 31844329.
7. Martinez M, Moon E K. CAR T Cells for Solid Tumors: New Strategies for Finding, Infiltrating, and Surviving in the Tumor Microenvironment. Front Immunol. 2019; 10:128. doi: 10.3389/fimmu.2019.00128. PubMed PMID: 30804938; PMCID: PMC6370640.
8. Labanieh L, Majzner R G, Mackall C L. Programming CAR-T cells to kill cancer. Nat Biomed Eng. 2018; 2 (6): 377-91. doi: 10.1038/s41551-018-0235-9. PubMed PMID: 31011197.
9. Russell J H, Ley T J. Lymphocyte-mediated cytotoxicity. Annu rev of immunol. 2002; 20:323-70. Epub 2002 Feb. 28. doi: 10.1146/annurev.immunol.20.100201.131730. PubMed PMID: 11861606.
10. Yang J, Pemberton A, Morrison W I, Connelley T. Granzyme B Is an Essential Mediator in CD8(+) T Cell Killing of Theileria parva-Infected Cells. Infect Immun. 2019; 87 (1). Epub 2018 Oct. 17. doi: 10.1128/iai.00386-18. PubMed PMID: 30323022; PMCID: PMC6300629.
11. Chávez-Galan L, Arenas-Del Angel M C, Zenteno E, Chávez R, Lascurain R. Cell Death Mechanisms Induced by Cytotoxic Lymphocytes. Cell Mol Immunol. 2009; 6 (1): 15-25. doi: 10.1038/cmi.2009.3.
12. Xie J, El Rami F, Zhou K, Simonetta F, Chen Z, Zheng X, Chen M, Balakrishnan P B, Dai S Y, Murty S, Alam I S, Baker J, Negrin R S, Gambhir S S, Rao J. Multiparameter Longitudinal Imaging of Immune Cell Activity in Chimeric Antigen Receptor T Cell and Checkpoint Blockade Therapies. ACS Cent Sci. 2022; 8 (5): 590-602. Epub 2022 Jun. 2. doi: 10.1021/acscentsci.2c00142. PubMed PMID: 35647285; PMCID: PMC9136971
13. Wherry E J, Kurachi M. Molecular and cellular insights into T cell exhaustion. Nat Rev Immunol. 2015; 15 (8): 486-99. Epub 2015 Jul. 25. doi: 10.1038/nri3862. PubMed PMID: 26205583; PMCID: 4889009.
14. B.T. D. Generation of an expression system for human granzyme B and analysis of the in vitro and in vivo efficiency [Dissertation]. Munich: Ludwig Maximilian University of Munich; 2010.
15. Yang O O, Lin H, Dagarag M, Ng H L, Effros R B, Uittenbogaart C H. Decreased perforin and granzyme B expression in senescent HIV-1-specific cytotoxic T lymphocytes. Virol. 2005; 332 (1): 16-9. Epub 2005 Jan. 22. doi: 10.1016/j.virol.2004.11.028. PubMed PMID: 15661136.
16. Miller B C, Sen D R, Al Abosy R, Bi K, Virkud Y V, LaFleur M W, Yates K B, Lako A, Felt K, Naik G S, Manos M, Gjini E, Kuchroo J R, Ishizuka J J, Collier J L, Griffin G K, Maleri S, Comstock D E, Weiss S A, Brown F D, Panda A, Zimmer M D, Manguso R T, Hodi F S, Rodig S J, Sharpe A H, Haining W N. Subsets of exhausted CD8(+) T cells differentially mediate tumor control and respond to checkpoint blockade. Nat Immunol. 2019; 20 (3): 326-36. Epub 2019 Feb. 20. doi: 10.1038/s41590-019-0312-6. PubMed PMID: 30778252; PMCID: 6673650.
17. Riches J C, Davies J K, McClanahan F, Fatah R, Iqbal S, Agrawal S, Ramsay A G, Gribben J G. T cells from CLL patients exhibit features of T-cell exhaustion but retain capacity for cytokine production. Blood. 2013; 121 (9): 1612-21. Epub 2012 Dec. 19. doi: 10.1182/blood-2012-09-457531. PubMed PMID: 23247726; PMCID: 3587324.
18. Wu X, Zhang H, Xing Q, Cui J, Li J, Li Y, Tan Y, Wang S. PD-1 (+) CD8(+) T cells are exhausted in tumours and functional in draining lymph nodes of colorectal cancer patients. Br J Cancer. 2014; 111 (7): 1391-9. Epub 2014 Aug. 6. doi: 10.1038/bjc.2014.416. PubMed PMID: 25093496; PMCID: 4183848.
19. Scott J I, Gutkin S, Green O, Thompson E J, Kitamura T, Shabat D, Vendrell M. A Functional Chemiluminescent Probe for in Vivo Imaging of Natural Killer Cell Activity Against Tumours. Angew Chem Int Ed Engl. 2021; 60 (11): 5699-703. Epub 2020 Dec. 11. doi: 10.1002/anie.202011429. PubMed PMID: 33300671; PMCID: PMC7986153.
20. Scott J I, Mendive-Tapia L, Gordon D, Barth N D, Thompson E J, Cheng Z, Taggart D, Kitamura T, Bravo-Blas A, Roberts E W, Juarez-Jimenez J, Michel J, Piet B, de Vries I J, Verdoes M, Dawson J, Carragher N O, Connor R A O, Akram A R, Frame M, Serrels A, Vendrell M. A fluorogenic probe for granzyme B enables in-biopsy evaluation and screening of response to anticancer immunotherapies. Nat Commun. 2022; 13 (1): 2366. Epub 2022 May 3. doi: 10.1038/s41467-022-29691-w. PubMed PMID: 35501326; PMCID: PMC9061857
21. He S, Li J, Lyu Y, Huang J, Pu K. Near-Infrared Fluorescent Macromolecular Reporters for Real-Time Imaging and Urinalysis of Cancer Immunotherapy. J Am Chem Soc. 2020; 142 (15): 7075-82. Epub 2020 Mar. 21. doi: 10.1021/jacs.0c00659. PubMed PMID: 32196318.
22. Xu L, Liu N, Zhan W, Deng Y, Chen Z, Liu X, Gao G, Chen Q, Liu Z, Liang G. Granzyme B Turns Nanoparticle Fluorescence “On” for Imaging Cytotoxic T Lymphocyte Activity in Vivo. ACS Nano. 2022; 16 (11): 19328-34. Epub 2022 Oct. 26. doi: 10.1021/acsnano.2c08896. PubMed PMID: 36282211.
23. Zhao N, Bardine C, Lourenço A L, Wang Y H, Huang Y, Cleary S J, Wilson D M, Oh D Y, Fong L, Looney M R, Evans M J, Craik C S. In Vivo Measurement of Granzyme Proteolysis from Activated Immune Cells with PET. ACS Cent Sci. 2021; 7 (10): 1638-49. Epub 2021 Nov. 4. doi: 10.1021/acscentsci. 1c00529. PubMed PMID: 34729407; PMCID: PMC8554823.
24. Rong J, Xu X, Ewen C, Bleackley R C, Kane K P. Isolation and characterization of novel single-chain Fv specific for human granzyme B. Hybrid Hybrid. 2004; 23 (4): 219-31. Epub 2004 Aug. 21. doi: 10.1089/1536859041651349. PubMed PMID: 15319069.
25. Guimaraes C P, Witte M D, Theile C S, Bozkurt G, Kundrat L, Blom A E M, Ploegh H L. Site-specific C-terminal and internal loop labeling of proteins using sortase-mediated reactions. Nat Protoc. 2013; 8 (9): 1787-99. doi: 10.1038/nprot.2013.101.
26. Rickner H D, Jiang L, Hong R, O'Neill N K, Mojica C A, Snyder B J, Zhang L, Shaw D, Medalla M, Wolozin B, Cheng C S. Single cell transcriptomic profiling of a neuron-astrocyte assembloid tauopathy model. Nat Commun. 2022; 13 (1): 6275. doi: 10.1038/s41467-022-34005-1.

### Example 2

### Design of AGB-⁸⁹Zr PET Tracer

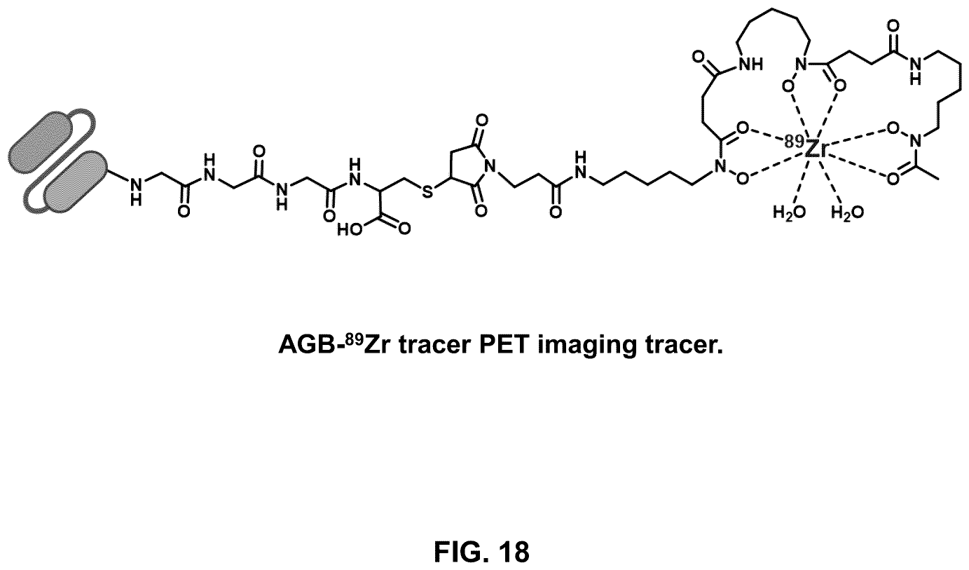

An AGB-⁸⁹Zr tracer for PET imaging was designed to predict tumor responses to immunotherapy in vivo. The tracer incorporates a chelator at AGB's C-terminus ((Gly) 3-DFO), enabling in vivo imaging of T cell-mediated cytotoxicity (FIG. 18).

### Synthesis of (Gly) 3-DFO

The tetrapeptide GGGC (SEQ ID NO:10) was purchased from Biosynth. Maleimide-DFO (purchased from Macrocyclics) was dissolved in DMSO. GGGC (SEQ ID NO: 10) was added and left to stir at room temperature for 30 min until LC-MS analysis indicated near-complete conversion to the product. The solution was filtered and purified by reverse-phase HPLC with a semipreparative column (C18 column, Hypersil GOLD, 5 μm, 10×250 mm; Thermo Scientific) at a flow rate of 5.0 ml/min;
solvent A: 0.1% TFA in H₂O, solvent B: 0.1% TFA in CH₃CN. (G) 3-DFO eluted at 70-75%;
solvent B: fractions containing pure product were collected and lyophilized.
   Preparing ⁸⁹Zr-Labeled AGB (⁸⁹Zr-AGB)

AGB-DFO was generated by SrtA mediated bio-ligation of (Gly) 3-DFO to AGB as previously described. A buffer solution of 0.5-2.0 mg of chelexed AGB-DFO in 200 μl of 0.5 M HEPES buffer, pH 7.5 was mixed with 300 μl ⁸⁹Zr⁴⁺ solution (1.0 M oxalic acid) corresponding to 1.0 to 1.5 mCi, pH 7.5. The reaction mixture was rotated at room temperature for 60 min. The labeled product was purified using a PD-10 size-exclusion cartridge (GE Healthcare), and eluted with PBS buffer, yielding >80% (~0.8-1.3 mCi).

### Example 3: A Rapid and Specific Fluorogenic Assay for Detecting Functional Tumor-Infiltrating Lymphocytes

### A. Overview

### TIL Therapy is a Promising New Treatment for Refractory Cancers, but its Success is Limited by Challenges in Identifying Functional TILs

Despite advances in checkpoint blockade and CAR T-cell therapies, only a subset of cancer responds to these treatments. Tumor-infiltrating lymphocyte (TIL) therapy, an emerging immunotherapeutic approach, offers distinct advantages. Unlike CAR T-cell therapy, which targets specific antigens, TILs recognize a diverse range of tumor neoantigens, making them effective against heterogeneous and antigen-loss tumors. Moreover, because TILs are naturally primed against a patient's tumor, they can overcome immune evasion mechanisms by attacking tumor cells that may have developed resistance to immune checkpoint inhibitors. Clinical trials have demonstrated durable responses and complete tumor regression in advanced diseases, suggesting long-term immune surveillance (Besser M J, Shapira-Frommer R, Itzhaki O, Treves A J, Zippel D B, Levy D, Kubi A, Shoshani N, Zikich D, Ohayon Y, Ohayon D, Shalmon B, Markel G, Yerushalmi R, Apter S, Ben-Nun A, Ben-Ami E, Shimoni A, Nagler A, Schachter J. Adoptive Transfer of Tumor-Infiltrating Lymphocytes in Patients with Metastatic Melanoma: Intent-to-Treat Analysis and Efficacy after Failure to Prior Immunotherapies. Clinical Cancer Research. 2013; 19 (17): 4792-800. doi: 10.1158/1078-0432.Ccr-13-0380). Furthermore, as TIL therapy uses autologous cells, it reduces the risk of graft-versus-host disease, a major complication of allogeneic cell therapies.

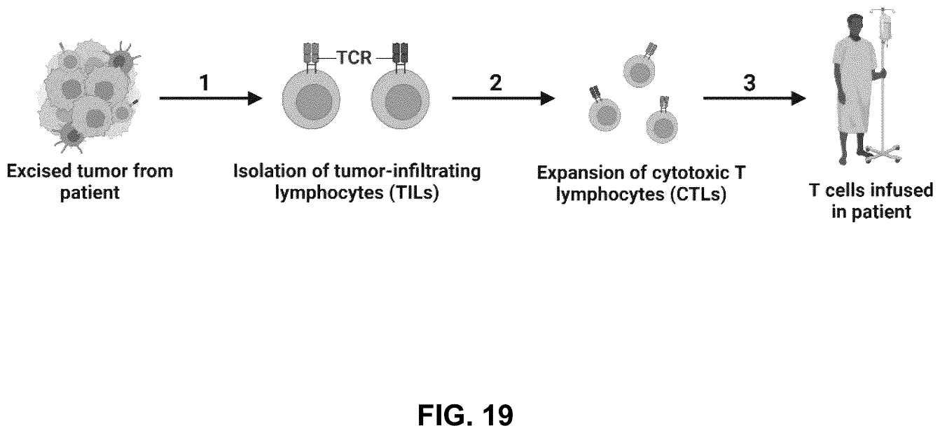

TIL therapy involves three key steps (FIG. 19): 1) Tumor resection and TILs isolation—A portion of the patient's tumor is surgically removed and fragmented into small pieces (~hundreds of cubic millimeters). Selected fragments are enzymatically digested to obtain single TILs, primarily cytotoxic T lymphocyte (CTLs); 2) TIL expansion—Isolated TILs are cultured and expanded ex vivo using high-dose interleukin-2 (IL-2) to enhance their proliferation and cytotoxic function. 3) Lymphodepletion and TIL infusion—Prior to TIL reinfusion, the patient undergoes a non-myeloablative lymphodepleting regimen to reduce immune suppression, creating a favorable environment for TIL persistence. Expanded TILs, only when reached 10-100 billion cells, are reinfused, followed by IL-2 administration to support their survival and activity.

While clinical studies and adoptive cell therapy protocols estimate that 10⁶ to 10⁷ TILs can be obtained per gram of tumor after enzymatic digestion (Crossey F, Marx S, Holters S, Schmitt K, Bohle R M, Schmidt T, Stockle M, Sester U, Sester M, Janssen M W W. Robust method for isolation of tumor infiltrating lymphocytes with a high vital cell yield from small samples of renal cell carcinomas by a new collagenase-free mechanical procedure. Urol Oncol. 2018; 36 (9): 402.e1-. e10. Epub 2018 Aug. 4. doi: 10.1016/j.urolonc.2018.06.002. PubMed PMID: 30072305; Fulbright O J, Forget M-A, Haymaker C, Bernatchez C. Isolation and Maintenance of Tumor-Infiltrating Lymphocytes for Translational and Clinical Applications: Established Methods and New Developments. In: McAllister F, editor. Cancer Immunoprevention: Methods and Protocols. New York, NY: Springer US; 2022. p. 43-71; Andersen R, Borch T H, Draghi A, Gokuldass A, Rana M A H, Pedersen M, Nielsen M, Kongsted P, Kjeldsen J W, Westergaard M C W, Radic H D, Chamberlain C A, Hölmich L R, Hendel H W, Larsen M S, Met O, Svane I M, Donia M. T cells isolated from patients with checkpoint inhibitor-resistant melanoma are functional and can mediate tumor regression. Annals of Oncology. 2018; 29 (7): 1575-81. doi: 10.1093/annonc/mdy139) up to 30-40% of early TIL cultures (pre-REP phase) from metastatic melanoma patients fail to meet the threshold for large-scale expansion (Besser M J, Shapira-Frommer R, Itzhaki O, Treves A J, Zippel D B, Levy D, Kubi A, Shoshani N, Zikich D, Ohayon Y, Ohayon D, Shalmon B, Markel G, Yerushalmi R, Apter S, Ben-Nun A, Ben-Ami E, Shimoni A, Nagler A, Schachter J. Adoptive Transfer of Tumor-Infiltrating Lymphocytes in Patients with Metastatic Melanoma: Intent-to-Treat Analysis and Efficacy after Failure to Prior Immunotherapies. Clinical Cancer Research. 2013; 19 (17): 4792-800. doi: 10.1158/1078-0432.Ccr-13-0380; Fulbright O J, Forget M-A, Haymaker C, Bernatchez C. Isolation and Maintenance of Tumor-Infiltrating Lymphocytes for Translational and Clinical Applications: Established Methods and New Developments. In: McAllister F, editor. Cancer Immunoprevention: Methods and Protocols. New York, NY: Springer US; 2022. p. 43-71). This high failure rate can be attributed to two main factors: 1) Heterogeneous distribution of TILs within tumors leads to low yields upon isolation; 2) Isolated TILs are not equally effective for cultural expansion due to the presence of terminally exhausted T lymphocytes—a deeply immunosuppressed TIL phenotype that has lost self-renewal capacity and effector function. These cells fail to proliferate effectively, limiting expansion success. Notably, TIL cultures from responding patients establish more quickly than those from nonresponders (Besser M J, Shapira-Frommer R, Itzhaki O, Treves A J, Zippel D B, Levy D, Kubi A, Shoshani N, Zikich D, Ohayon Y, Ohayon D, Shalmon B, Markel G, Yerushalmi R, Apter S, Ben-Nun A, Ben-Ami E, Shimoni A, Nagler A, Schachter J. Adoptive Transfer of Tumor-Infiltrating Lymphocytes in Patients with Metastatic Melanoma: Intent-to-Treat Analysis and Efficacy after Failure to Prior Immunotherapies. Clinical Cancer Research. 2013; 19 (17): 4792-800. doi: 10.1158/1078-0432.Ccr-13-0380; Besser M J, Shapira-Frommer R, Treves A J, Zippel D, Itzhaki O, Hershkovitz L, Levy D, Kubi A, Hovav E, Chermoshniuk N, Shalmon B, Hardan I, Catane R, Markel G, Apter S, Ben-Nun A, Kuchuk I, Shimoni A, Nagler A, Schachter J. Clinical Responses in a Phase II Study Using Adoptive Transfer of Short-term Cultured Tumor Infiltration Lymphocytes in Metastatic Melanoma Patients. Clinical Cancer Research. 2010; 16 (9): 2646-55. doi: 10.1158/1078-0432.Ccr-10-0041). Therefore, early identification of functional TILs, rather than isolating and expanding exhausted populations, could streamline expansion process, shorten culture times, improve success rates, and enhance therapeutic efficacy.

### Limitations of Current Functional TIL Detection Methods

Existing methods for detecting functional TILs have significant drawbacks. Histological staining (IHC, IF) provides spatial localization of TILs but is inherently destructive. Flow cytometry allows multiparametric functional analysis but requires prior tissue dissociation. The high fluidic pressure can often stress T cells, reducing their viability and proliferation capacity. PCR-based techniques detect T cell-related gene expression but do not assess actual function. Moreover, these methods analyze only small aliquots of tumor resections, extrapolating findings that may not be representative due to tumor heterogeneity. There is an urgent need for a non-destructive, high-throughput method capable of screening multiple tumor fragments simultaneously, identifying functional TILs without disrupting their viability.

### B. A Fluorogenic Assay for Rapid, Specific, and High-Throughput Screening of Functional TILs

To address this critical challenge, an innovative fluorogenic assay is described that integrates seamlessly into Step 1 of TIL therapy (FIG. 19) to screen all fragmented tumor resections and identify functional cytotoxic T lymphocyte (CTLs) within approximately one hour.

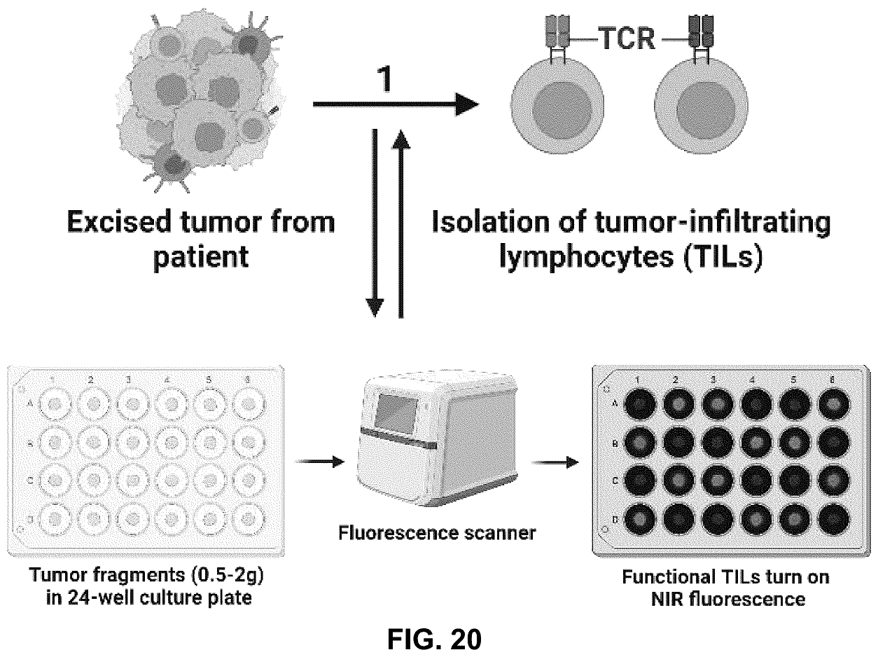

As illustrated in FIG. 20, tumor resections are fragmented into 0.5-2 g pieces and cultured in 24-well plates with AGB-CyP, a fluorogenic probe designed to report CTL cytotoxic function via fluorescence activation. After a one-hour incubation at 37° C., the plates are scanned using a fluorescence imager to quantify near-infrared (NIR) fluorescence. The fluorescence intensity correlates directly with the presence of functional CTLs, allowing for the rapid identification of tumor fragments that should be prioritized for immediate enzymatic digestion and TIL isolation. Compared to existing methods, this product offers a rapid, specific, and high-throughput screening assay based on fluorescence imaging to identify functional TILs, particularly CTLs, from patients' tumor resections for effective TIL therapy.

### Market Overview

In February 2024, the U.S. Food and Drug Administration (FDA) approved Amtagvi (lifileucel), the first tumor-infiltrating lymphocyte (TIL)-based therapy developed by lovance Biotherapeutics. This approval marks a significant milestone for TIL therapy, providing a new treatment option for patients with metastatic or unresectable melanoma who have progressed on standard immune checkpoint inhibitors and BRAF-targeted therapies.

As of March 2024, there are 96 ongoing clinical trials worldwide evaluating TIL therapies across various cancer types. The initiation of phase III trials increased by 33.3% in 2022 and 43.8% in 2023, indicating ongoing advancement in clinical development. The global TIL therapy market is projected to reach $9.2 billion by 2031, with the number of patients receiving TIL therapy expected to surpass one million by 2034. This growing demand highlights the need for efficient and scalable solutions to optimize TIL therapy, such fluorogenic assays for functional TIL screening described herein.

### C. Probe Design and Assay Development Progress

AGB-CyP has been developed as described herein, a fluorogenic probe designed to detect functional tumor-infiltrating lymphocytes (TILs) through granzyme B (GzmB) release. Data herein confirm its feasibility for high-throughput screening in laboratory settings, currently validated at NIH Technology Readiness Level (TRL) 3.

### Granzyme B as a Biomarker of Cytotoxic T Cell Function

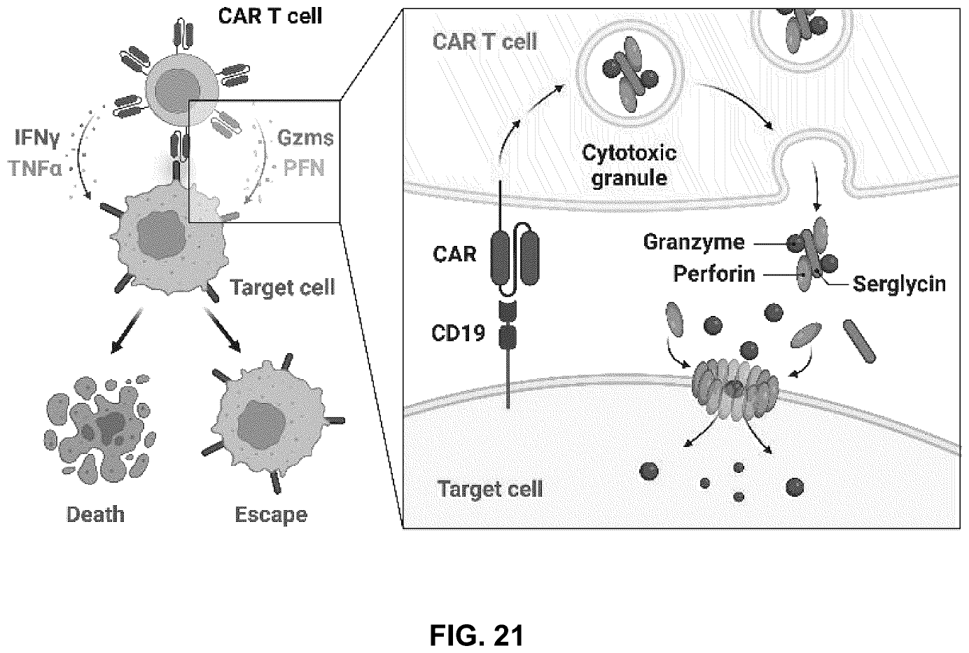

Cytotoxic T lymphocytes (CTLs) eliminate cancer cells by releasing cytotoxic granules containing perforin and granzyme B (GzmB) upon antigen activation (e.g., CAR T cells, FIG. 21). Perforin forms pores in the cancer cell membrane, allowing GzmB, a potent serine protease, to enter and induce programmed cell death. Loss of GzmB has been shown to reduce cytotoxic efficacy by 50-70% (Yang J, Pemberton A, Morrison W I, Connelley T. Granzyme B Is an Essential Mediator in CD8(+) T Cell Killing of Theileria parva-Infected Cells. Infect Immun. 2019; 87 (1). Epub 2018 Oct. 17. doi: 10.1128/iai.00386-18. PubMed PMID: 30323022; PMCID: PMC6300629).

In vitro and vivo studies have established degranulation and GzmB release as reliable biomarkers of CTL function (Xie J, El Rami F, Zhou K, Simonetta F, Chen Z, Zheng X, Chen M, Balakrishnan P B, Dai S Y, Murty S, Alam I S, Baker J, Negrin R S, Gambhir S S, Rao J. Multiparameter Longitudinal Imaging of Immune Cell Activity in Chimeric Antigen Receptor T Cell and Checkpoint Blockade Therapies. ACS Cent Sci. 2022; 8 (5): 590-602. Epub 2022 Jun. 2. doi: 10.1021/acscentsci.2c00142. PubMed PMID: 35647285; PMCID: PMC9136971; Wherry E J, Kurachi M. Molecular and cellular insights into T cell exhaustion. Nat Rev Immunol. 2015; 15 (8): 486-99. Epub 2015 Jul. 25. doi: 10.1038/nri3862. PubMed PMID: 26205583; PMCID: 4889009). Notably, GzmB remains stable and retains catalytic activity even in the acidic environment of cytotoxic granules before release (B.T. D. Generation of an expression system for human granzyme B and analysis of the in vitro and in vivo efficiency [Dissertation]. Munich: Ludwig Maximilian University of Munich; 2010). However, terminally exhausted T cells, often found in advanced tumors, express high levels of intracellular GzmB despite being functionally impaired (Miller B C, Sen D R, Al Abosy R, Bi K, Virkud Y V, LaFleur M W, Yates K B, Lako A, Felt K, Naik G S, Manos M, Gjini E, Kuchroo J R, Ishizuka J J, Collier J L, Griffin G K, Maleri S, Comstock D E, Weiss S A, Brown F D, Panda A, Zimmer M D, Manguso R T, Hodi F S, Rodig S J, Sharpe A H, Haining W N. Subsets of exhausted CD8(+) T cells differentially mediate tumor control and respond to checkpoint blockade. Nat Immunol. 2019; 20 (3): 326-36. Epub 2019 Feb. 20. doi: 10.1038/s41590-019-0312-6. PubMed PMID: 30778252; PMCID: 6673650; Riches J C, Davies J K, McClanahan F, Fatah R, Iqbal S, Agrawal S, Ramsay A G, Gribben J G. T cells from CLL patients exhibit features of T-cell exhaustion but retain capacity for cytokine production. Blood. 2013; 121 (9): 1612-21. Epub 2012 Dec. 19. doi: 10.1182/blood-2012-09-457531. PubMed PMID: 23247726; PMCID: 3587324; Wu X, Zhang H, Xing Q, Cui J, Li J, Li Y, Tan Y, Wang S. PD-1(+) CD8(+) T cells are exhausted in tumours and functional in draining lymph nodes of colorectal cancer patients. Br J Cancer. 2014; 111 (7): 1391-9. Epub 2014 Aug. 6. doi: 10.1038/bjc.2014.416. PubMed PMID: 25093496; PMCID: 4183848). As a result, existing imaging probes, which detect both intracellular and extracellular GzmB, cannot distinguish between functional and exhausted CTLs (Xie J, El Rami F, Zhou K, Simonetta F, Chen Z, Zheng X, Chen M, Balakrishnan P B, Dai S Y, Murty S, Alam I S, Baker J, Negrin R S, Gambhir S S, Rao J. Multiparameter Longitudinal Imaging of Immune Cell Activity in Chimeric Antigen Receptor T Cell and Checkpoint Blockade Therapies. ACS Cent Sci. 2022; 8 (5): 590-602. Epub 2022 Jun. 2. doi: 10.1021/acscentsci.2c00142. PubMed PMID: 35647285; PMCID: PMC9136971; Scott J I, Gutkin S, Green O, Thompson E J, Kitamura T, Shabat D, Vendrell M. A Functional Chemiluminescent Probe for in Vivo Imaging of Natural Killer Cell Activity Against Tumours. Angew Chem Int Ed Engl. 2021; 60 (11): 5699-703. Epub 2020 Dec. 11. doi: 10.1002/anie.202011429. PubMed PMID: 33300671; PMCID: PMC7986153; Scott J I, Mendive-Tapia L, Gordon D, Barth N D, Thompson E J, Cheng Z, Taggart D, Kitamura T, Bravo-Blas A, Roberts E W, Juarez-Jimenez J, Michel J, Piet B, de Vries I J, Verdoes M, Dawson J, Carragher N O, Connor R A O, Akram A R, Frame M, Serrels A, Vendrell M. A fluorogenic probe for granzyme B enables in-biopsy evaluation and screening of response to anticancer immunotherapies. Nat Commun. 2022; 13 (1): 2366. Epub 2022 May 3. doi: 10.1038/s41467-022-29691-w. PubMed PMID: 35501326; PMCID: PMC9061857; He S, Li J, Lyu Y, Huang J, Pu K. Near-Infrared Fluorescent Macromolecular Reporters for Real-Time Imaging and Urinalysis of Cancer Immunotherapy. J Am Chem Soc. 2020; 142 (15): 7075-82. Epub 2020 Mar. 21. doi: 10.1021/jacs.0c00659. PubMed PMID: 32196318; Xu L, Liu N, Zhan W, Deng Y, Chen Z, Liu X, Gao G, Chen Q, Liu Z, Liang G. Granzyme B Turns Nanoparticle Fluorescence “On” for Imaging Cytotoxic T Lymphocyte Activity in Vivo. ACS Nano. 2022; 16 (11): 19328-34. Epub 2022 Oct. 26. doi: 10.1021/acsnano.2c08896. PubMed PMID: 36282211; Zhao N, Bardine C, Lourenço A L, Wang Y H, Huang Y, Cleary S J, Wilson D M, Oh D Y, Fong L, Looney M R, Evans M J, Craik C S. In Vivo Measurement of Granzyme Proteolysis from Activated Immune Cells with PET. ACS Cent Sci. 2021; 7 (10): 1638-49. Epub 2021 Nov. 4. doi: 10.1021/acscentsci. 1c00529. PubMed PMID: 34729407; PMCID: PMC8554823).

### Design of AGB-CyP: A Fluorogenic Probe for Imaging CTL Function

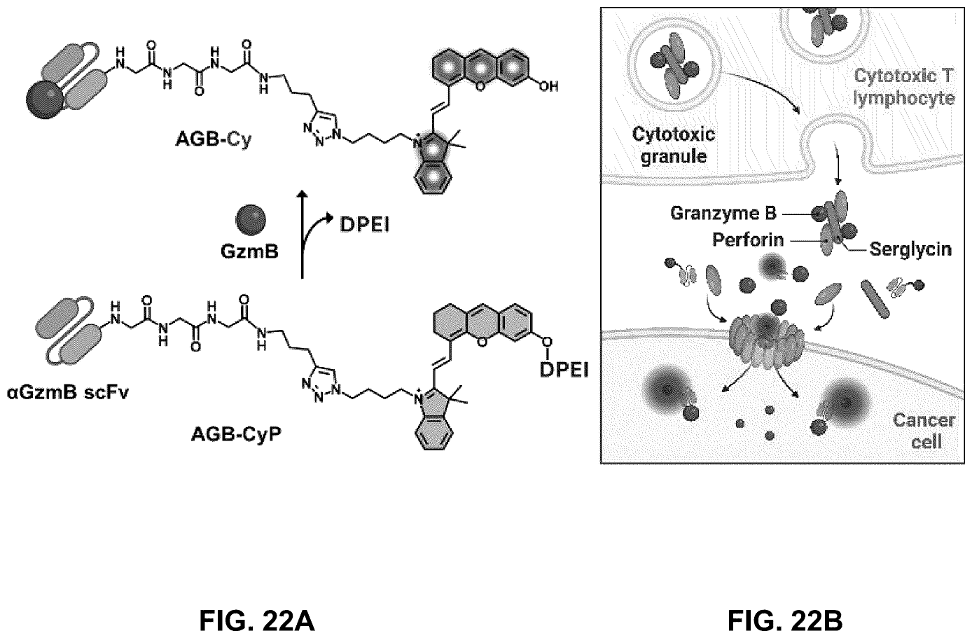

To overcome these limitations, AGB-CyP was designed, a fluorogenic probe that selectively detects extracellular GzmB. AGB-CyP integrates a high-affinity scFv antibody (AGB) targeting GzmB with an IEPD peptide (SEQ ID NO:8)-caged near-infrared fluorochrome (CyP) (FIG. 22A). The IEPD peptide (SEQ ID NO:8) serves as a substrate specific to human GzmB, ensuring fluorescence activation only by released GzmB.

AGB-CyP is non-cell-permeable, preventing detection of intracellular GzmB within CTLs. Instead, during active degranulation, the probe binds and is activated by extracellular GzmB, selectively labeling cancer cells undergoing cytotoxicity (FIG. 22B). This mechanism allows real-time imaging of functional CTLs while distinguishing them from terminally exhausted CTLs, which retain intracellular GzmB but lack effective cytotoxic function.

### Generation and Validation of AGB-CyP

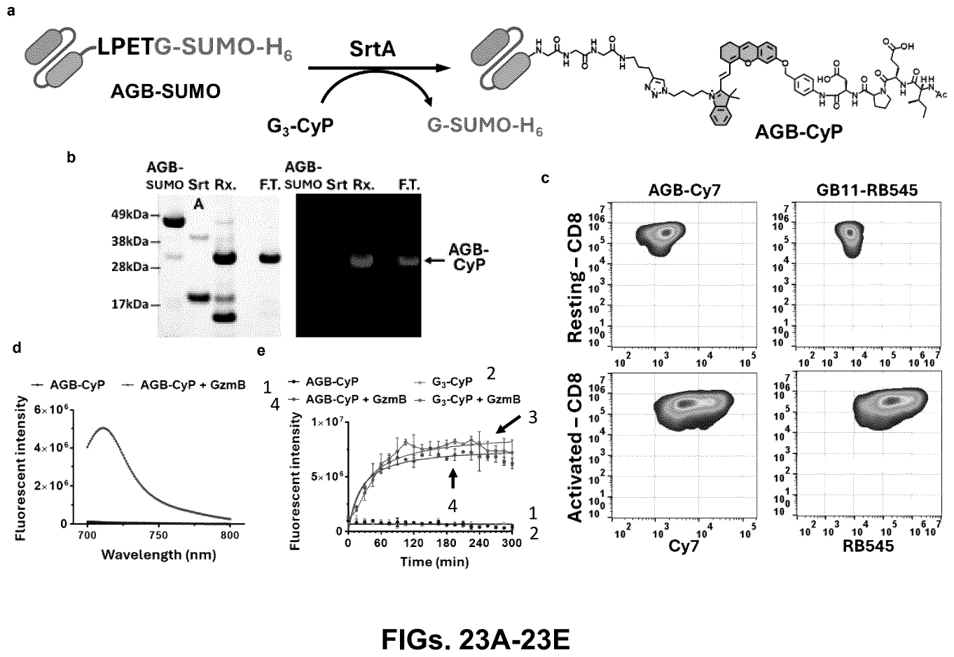

AGB-CyP was synthesized using sortase A (SrtA)-mediated bio-ligation (Guimaraes C P, Witte M D, Theile C S, Bozkurt G, Kundrat L, Blom A E M, Ploegh H L. Site-specific C-terminal and internal loop labeling of proteins using sortase-mediated reactions. Nature Protocols. 2013; 8 (9): 1787-99. doi: 10.1038/nprot.2013.101), linking the anti-GzmB scFv antibody to a fluorogenic CyP compound (G3-CyP) (FIG. 23A). The scFv antibodies and fluorogenic compounds were successfully purified and synthesized for research-scale studies. The final probe was validated through affinity (FIG. 23B) and binding assays (FIG. 23C), demonstrating strong specificity for GzmB.

### Enhanced Imaging Properties of AGB-CyP

Fluorescence imaging in tissue is often hindered by background autofluorescence, light scattering, and limited penetration depth. AGB-CyP addresses these challenges through near-infrared fluorescence activation. Before GzmB activation, AGB-CyP exhibits dual excitation peaks at 610 nm and 660 nm, with weak emission at 675 nm. Upon activation, fluorescence red shifts to near-infrared Ex690/Em715, yielding a ~60-fold signal increase (FIG. 23D). These properties enable high-contrast, deep-tissue imaging suitable for tumor resections. Importantly, AGB-CyP binding does not interfere with GzmB's catalytic activity (FIG. 23D).

AGB-CyP Selectively Labels Released GzmB from Functional CTLs

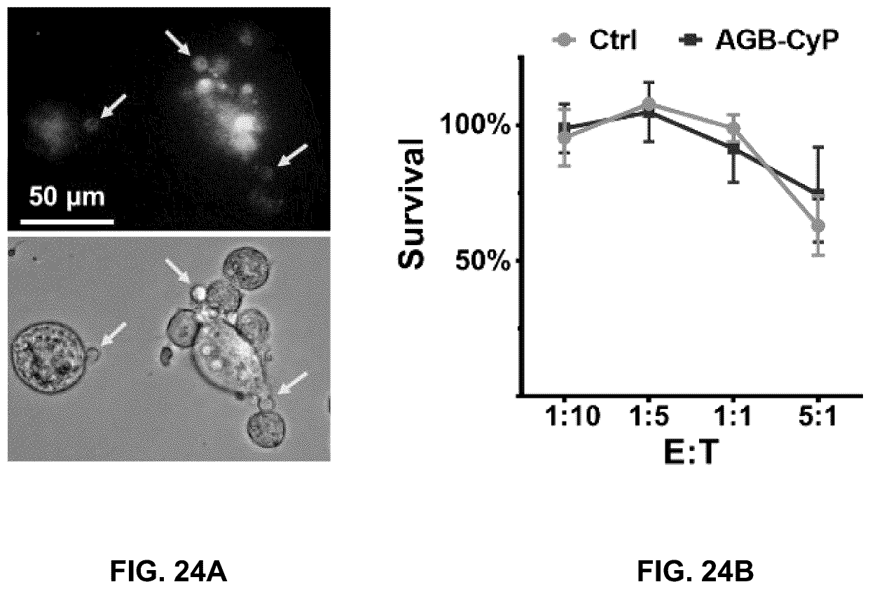

To confirm if AGB-CyP could be activated and label released GzmB during functional T cell mediated cytotoxicity, GFP-expressing CD19-28ζ CAR T cells were engineered from a healthy donor and incubated at 1:1 ratio overnight with glioblastoma cells (U87 MG) expressing CD19 in the presence of AGB-CyP. As shown in FIG. 24A, only apoptotic cancer cells engaged by CAR T cells exhibited red fluorescence, confirming AGB-CyP activation by released GzmB. In comparison, no red fluorescence was observed in T cells (yellow arrows). These cell imaging data validated that AGB-CyP reports only released GzmB from functional T cells during cytotoxic cancer cell killing.

### AGB-CyP is Safe for T Cell Imaging

To evaluate potential cytotoxic effects of AGB-CyP on T cell function, GFP CAR T cells were incubated with luciferase-expressing lymphoma cells (Raji-Fluc) in the presence of 200 nM AGB-CyP. A bioluminescence assay after 24 hours confirmed no reduction in CAR T cell viability or cytotoxic activity (FIG. 24B), demonstrating AGB-CyP's biocompatibility and suitability for clinical translation.

### AGB-CyP Detects TILs in 3D Tumor Models and Tissue Phantoms Mimicking Fragmented Tumor Resections

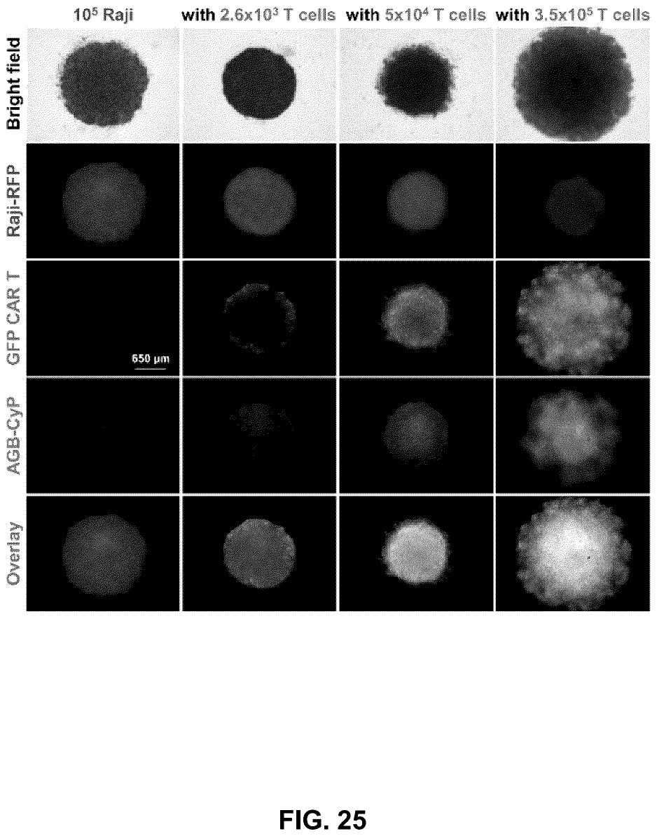

The sensitivity of AGB-CyP was further evaluated in 3D tumor spheroids and tissue phantom models, which better mimic the tumor microenvironment compared to 2D cultures. Lymphoma tumor spheroids were generated using Raji-RFP cells and treated with varying numbers of GFP-expressing CD19-28ζ CAR T cells. After 96 hours, AGB-CyP incubation and imaging confirmed successful detection of as few as 2.6×10³ infiltrated CAR T cells (FIG. 25).

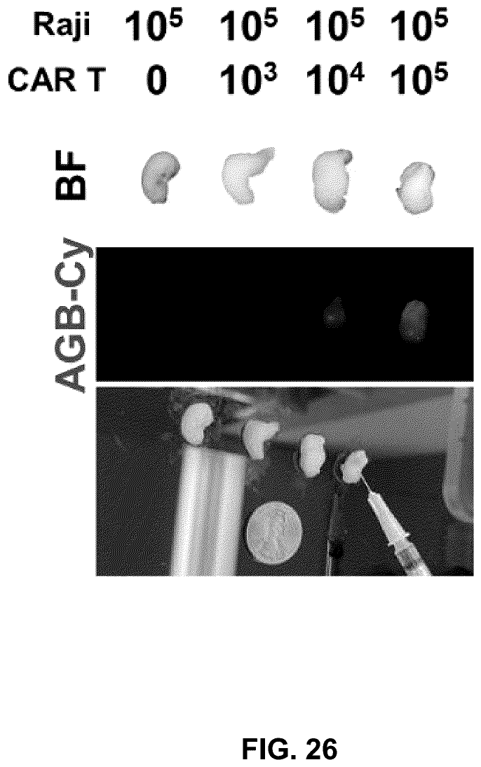

To simulate tumor resections, CAR T cell-treated spheroids were resuspended in PBS containing 200 nM AGB-CyP and injected into fragmented chicken breast tissue (0.2-0.3 g per piece). Fluorescence imaging after one hour revealed detection of as few as 104 CAR T cells (FIG. 26). These findings highlight AGB-CyP's superior sensitivity, exceeding the clinically estimated 10⁶-10⁷ TILs per gram of tumor.

Together, these data validate the design of AGB-CyP, demonstrating its superior sensitivity over clinically estimated 10⁶ to 10⁷ TILs per gram of tumor. These findings strongly support the further development of AGB-CyP for TIL imaging applications.

### D. Milestones

### Recombinant AGB Expression and Purification

Approximately 1.5 mg of AGB can be expressed and purified from a one-liter bacterial culture using two rounds of forward and reverse affinity purification, yielding 1 mg (24 nmol) of final AGB-CyP product.

### Differentiating Exhausted and Functional TILs in Tumor Spheroid Models

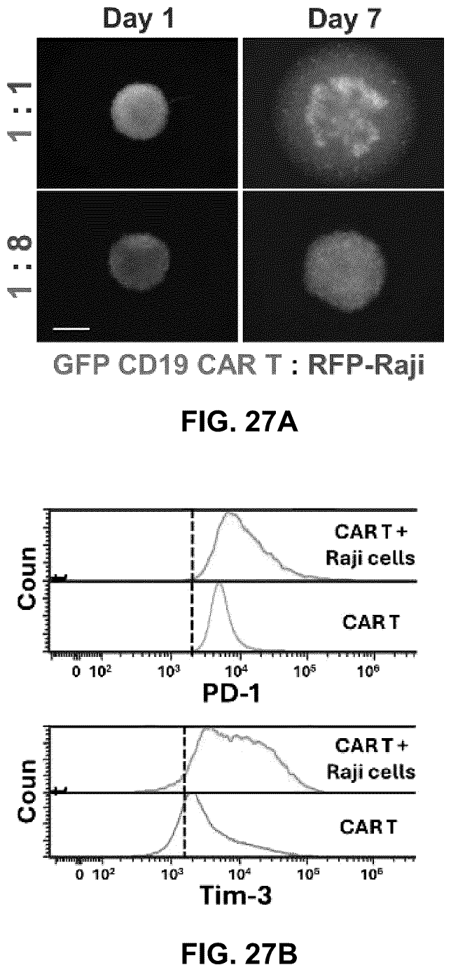

A notable feature of the fluorogenic assay of the present example is its ability to selectively report functional cytotoxic T lymphocytes (CTLs) in tumor resections. To validate this, CAR T cell exhaustion can be induced by modifying the effector (CAR T)-to-target (Raji) ratio as has been demonstrated (FIGS. 27A, 27B). Deeply exhausted CAR T cells can be collected, and they can be allowed to infiltrate newly established tumor spheroids, and their fluorescence readout can be compared to that of fully functional TILs.

### E. References Related to the Present Example

1. Besser M J, Shapira-Frommer R, Itzhaki O, Treves A J, Zippel D B, Levy D, Kubi A, Shoshani N, Zikich D, Ohayon Y, Ohayon D, Shalmon B, Markel G, Yerushalmi R, Apter S, Ben-Nun A, Ben-Ami E, Shimoni A, Nagler A, Schachter J. Adoptive Transfer of Tumor-Infiltrating Lymphocytes in Patients with Metastatic Melanoma: Intent-to-Treat Analysis and Efficacy after Failure to Prior Immunotherapies. Clinical Cancer Research. 2013; 19 (17): 4792-800. doi: 10.1158/1078-0432.Ccr-13-0380.
2. Crossey F, Marx S, Holters S, Schmitt K, Bohle R M, Schmidt T, Stockle M, Sester U, Sester M, Janssen M W W. Robust method for isolation of tumor infiltrating lymphocytes with a high vital cell yield from small samples of renal cell carcinomas by a new collagenase-free mechanical procedure. Urol Oncol. 2018; 36 (9): 402.e1-. e10. Epub 2018 Aug. 4. doi: 10.1016/j.urolonc.2018.06.002. PubMed PMID: 30072305.
3. Fulbright O J, Forget M-A, Haymaker C, Bernatchez C. Isolation and Maintenance of Tumor-Infiltrating Lymphocytes for Translational and Clinical Applications: Established Methods and New Developments. In: McAllister F, editor. Cancer Immunoprevention: Methods and Protocols. New York, NY: Springer US; 2022. p. 43-71.
4. Andersen R, Borch T H, Draghi A, Gokuldass A, Rana M A H, Pedersen M, Nielsen M, Kongsted P, Kjeldsen J W, Westergaard M C W, Radic H D, Chamberlain C A, Holmich L R, Hendel H W, Larsen M S, Met O, Svane I M, Donia M. T cells isolated from patients with checkpoint inhibitor-resistant melanoma are functional and can mediate tumor regression. Annals of Oncology. 2018; 29 (7): 1575-81. doi: 10.1093/annonc/mdyl39.
5. Besser M J, Shapira-Frommer R, Treves A J, Zippel D, Itzhaki O, Hershkovitz L, Levy D, Kubi A, Hovav E, Chermoshniuk N, Shalmon B, Hardan I, Catane R, Markel G, Apter S, Ben-Nun A, Kuchuk I, Shimoni A, Nagler A, Schachter J. Clinical Responses in a Phase II Study Using Adoptive Transfer of Short-term Cultured Tumor Infiltration Lymphocytes in Metastatic Melanoma Patients. Clinical Cancer Research. 2010; 16 (9): 2646-55. doi: 10.1158/1078-0432.Ccr-10-0041.
6. Yang J, Pemberton A, Morrison W I, Connelley T. Granzyme B Is an Essential Mediator in CD8(+) T Cell Killing of Theileria parva-Infected Cells. Infect Immun. 2019; 87 (1). Epub 2018 Oct. 17. doi: 10.1128/iai.00386-18. PubMed PMID: 30323022; PMCID: PMC6300629.
7. Xie J, El Rami F, Zhou K, Simonetta F, Chen Z, Zheng X, Chen M, Balakrishnan P B, Dai S Y, Murty S, Alam I S, Baker J, Negrin R S, Gambhir S S, Rao J. Multiparameter Longitudinal Imaging of Immune Cell Activity in Chimeric Antigen Receptor T Cell and Checkpoint Blockade Therapies. ACS Cent Sci. 2022; 8 (5): 590-602. Epub 2022 Jun. 2. doi: 10.1021/acscentsci.2c00142. PubMed PMID: 35647285; PMCID: PMC9136971.
8. Wherry E J, Kurachi M. Molecular and cellular insights into T cell exhaustion. Nat Rev Immunol. 2015; 15 (8): 486-99. Epub 2015 Jul. 25. doi: 10.1038/nri3862. PubMed PMID: 26205583; PMCID: 4889009.
9. B.T. D. Generation of an expression system for human granzyme B and analysis of the in vitro and in vivo efficiency [Dissertation]. Munich: Ludwig Maximilian University of Munich; 2010.
10. Miller B C, Sen D R, Al Abosy R, Bi K, Virkud Y V, LaFleur M W, Yates K B, Lako A, Felt K, Naik G S, Manos M, Gjini E, Kuchroo J R, Ishizuka J J, Collier J L, Griffin G K, Maleri S, Comstock D E, Weiss S A, Brown F D, Panda A, Zimmer M D, Manguso R T, Hodi F S, Rodig S J, Sharpe A H, Haining W N. Subsets of exhausted CD8(+) T cells differentially mediate tumor control and respond to checkpoint blockade. Nat Immunol. 2019; 20 (3): 326-36. Epub 2019 Feb. 20. doi: 10.1038/s41590-019-0312-6. PubMed PMID: 30778252; PMCID: 6673650.
11. Riches J C, Davies J K, McClanahan F, Fatah R, Iqbal S, Agrawal S, Ramsay A G, Gribben J G. T cells from CLL patients exhibit features of T-cell exhaustion but retain capacity for cytokine production. Blood. 2013; 121 (9): 1612-21. Epub 2012 Dec. 19. doi: 10.1182/blood-2012-09-457531. PubMed PMID: 23247726; PMCID: 3587324.
12. Wu X, Zhang H, Xing Q, Cui J, Li J, Li Y, Tan Y, Wang S. PD-1 (+) CD8(+) T cells are exhausted in tumours and functional in draining lymph nodes of colorectal cancer patients. Br J Cancer. 2014; 111 (7): 1391-9. Epub 2014 Aug. 6. doi: 10.1038/bjc.2014.416. PubMed PMID: 25093496; PMCID: 4183848.
13. Scott J I, Gutkin S, Green O, Thompson E J, Kitamura T, Shabat D, Vendrell M. A Functional Chemiluminescent Probe for in Vivo Imaging of Natural Killer Cell Activity Against Tumours. Angew Chem Int Ed Engl. 2021; 60 (11): 5699-703. Epub 2020 Dec. 11. doi: 10.1002/anie.202011429. PubMed PMID: 33300671; PMCID: PMC7986153.
14. Scott J I, Mendive-Tapia L, Gordon D, Barth N D, Thompson E J, Cheng Z, Taggart D, Kitamura T, Bravo-Blas A, Roberts E W, Juarez-Jimenez J, Michel J, Piet B, de Vries I J, Verdoes M, Dawson J, Carragher N O, Connor R A O, Akram A R, Frame M, Serrels A, Vendrell M. A fluorogenic probe for granzyme B enables in-biopsy evaluation and screening of response to anticancer immunotherapies. Nat Commun. 2022; 13 (1): 2366. Epub 2022 May 3. doi: 10.1038/s41467-022-29691-w. PubMed PMID: 35501326; PMCID: PMC9061857 described in this manuscript.
15. He S, Li J, Lyu Y, Huang J, Pu K. Near-Infrared Fluorescent Macromolecular Reporters for Real-Time Imaging and Urinalysis of Cancer Immunotherapy. J Am Chem Soc. 2020; 142 (15): 7075-82. Epub 2020 Mar. 21. doi: 10.1021/jacs.0c00659. PubMed PMID: 32196318.
16. Xu L, Liu N, Zhan W, Deng Y, Chen Z, Liu X, Gao G, Chen Q, Liu Z, Liang G. Granzyme B Turns Nanoparticle Fluorescence “On” for Imaging Cytotoxic T Lymphocyte Activity in Vivo. ACS Nano. 2022; 16 (11): 19328-34. Epub 2022 Oct. 26. doi: 10.1021/acsnano.2c08896. PubMed PMID: 36282211.
17. Zhao N, Bardine C, Lourenço A L, Wang Y H, Huang Y, Cleary S J, Wilson D M, Oh D Y, Fong L, Looney M R, Evans M J, Craik C S. In Vivo Measurement of Granzyme Proteolysis from Activated Immune Cells with PET. ACS Cent Sci. 2021; 7 (10): 1638-49. Epub 2021 Nov. 4. doi: 10.1021/acscentsci. 1c00529. PubMed PMID: 34729407; PMCID: PMC8554823.
18. Guimaraes C P, Witte M D, Theile C S, Bozkurt G, Kundrat L, Blom A E M, Ploegh H L. Site-specific C-terminal and internal loop labeling of proteins using sortase-mediated reactions. Nature Protocols. 2013; 8 (9): 1787-99. doi: 10.1038/nprot.2013.101.
19. Itzhaki O, Hovav E, Ziporen Y, Levy D, Kubi A, Zikich D, Hershkovitz L, Treves A J, Shalmon B, Zippel D, Markel G, Shapira-Frommer R, Schachter J, Besser M J. Establishment and large-scale expansion of minimally cultured “young” tumor infiltrating lymphocytes for adoptive transfer therapy. J Immunother. 2011; 34 (2): 212-20. Epub 2011 Feb. 10. doi: 10.1097/CJI.0b013e318209c94c. PubMed PMID: 21304398.

### Example 4: Rapid Functional Imaging of Cancer-Reactive Tumor-Infiltrating Lymphocytes in Intact Surgical Resections

### A. Overview

Patients with advanced solid tumors face poor outcomes despite intensive multimodal therapies. Tumor-infiltrating lymphocyte (TIL) therapy offers curative potential, yet its clinical success is limited by a fundamental bottleneck: there is no practical method to determine whether surgical tumor resections contain functional, cancer-reactive TILs prior to manufacturing. Consequently, patients often undergo costly and time-consuming TIL manufacturing processes without assurance that effective therapeutic products can be generated, and a substantial fraction ultimately fail to benefit. Here a near-infrared fluorogenic single-chain variable fragment (scFv) probe is reported, AGB-CyP, that enables rapid and specific detection of functional TILs by selectively reporting degranulated granzyme B (GzmB) during cytotoxic T lymphocytes (CTLs)-mediated tumor cell killing. Immunofluorescence analysis of melanoma resections from 11 patients revealed concentrated and robust degranulation at tumor sites infiltrated by CTLs, with the majority of released GzmB taken up by CTL-engaged tumor cells and a smaller fraction remaining extracellular. Guided by these observations, AGB-CyP was rationally designed to transit perforin pores to maximize detection sensitivity while remaining non-permeable to intact T cells. In two-dimensional co-culture and three-dimensional tumor spheroid chimeric antigen receptor (CAR) T-cell therapy models, AGB-CyP was activated exclusively by GzmB released from functional T cells, reporting cytotoxic activity while avoiding detection of intracellular GzmB retained by terminally exhausted T cells. Application of AGB-CyP to melanoma surgical resections enabled sensitive, specific, and high-throughput identification of functional, cancer-reactive TILs in intact tumor specimens. Together, this fluorogenic probe enables rapid, non-destructive functional imaging of TIL activity directly in surgical tumor resections, with the potential to reduce manufacturing failures, lower costs, and improve patient selection and therapeutic outcomes.

This example reports on the development of fluorogenic imaging of granzyme B activity to enable non-destructive detection of functional tumor-infiltrating lymphocytes.

### B. Introduction

Tumor-infiltrating lymphocyte (TIL) therapy, first approved in 2024, offers curative potential for patients with otherwise hard-to-treat advanced solid tumors. Unlike checkpoint blockade or chimeric antigen receptor (CAR) T-cell therapies that target predefined antigens, the cytotoxic T lymphocytes (CTLs) in TIL products are polyclonal and recognize a broad repertoire of neoantigens, rendering them effective against heterogeneous and antigen-loss malignancies (S. A. Rosenberg, P. Spiess, R. Lafreniere, A new approach to the adoptive immunotherapy of cancer with tumor-infiltrating lymphocytes. Science 233, 1318-1321 (1986)). In advanced metastatic melanoma, TIL therapy has demonstrated overall response rates of 30-50%, with durable complete response in up to 20% of heavily pre-treated patients, including those refractory to checkpoint inhibitors and BRAF-targeted therapies (A. Betof Warner, P. G. Corrie, O. Hamid, Tumor-Infiltrating Lymphocyte Therapy in Melanoma: Facts to the Future. Clinical Cancer Research 29, 1835-1854 (2023)).

TILs are manufactured by expanding lymphocytes ex vivo from surgically resected tumor tissues for reinfusion into the patient (A. Betof Warner, P. G. Corrie, O. Hamid, Tumor-Infiltrating Lymphocyte Therapy in Melanoma: Facts to the Future. Clinical Cancer Research 29, 1835-1854 (2023)). Although clinical studies and adoptive cell therapy protocols estimate that 10⁶ to 10⁷ TILs can be obtained per gram of tumor tissue on average (3-5), TIL manufacturing fails in 5-41% of cases, depending on tumor type (A. Betof Warner, P. G. Corrie, O. Hamid, Tumor-Infiltrating Lymphocyte Therapy in Melanoma: Facts to the Future. Clinical Cancer Research 29, 1835-1854 (2023); 4. O. J. Fulbright, M.-A. Forget, C. Haymaker, C. Bernatchez, in Cancer Immunoprevention: Methods and Protocols, F. McAllister, Ed. (Springer US, New York, NY, 2022), pp. 43-71; M. J. Besser, R. Shapira-Frommer, O. Itzhaki, A. J. Treves, D. B. Zippel, D. Levy, A. Kubi, N. Shoshani, D. Zikich, Y. Ohayon, D. Ohayon, B. Shalmon, G. Markel, R. Yerushalmi, S. Apter, A. Ben-Nun, E. Ben-Ami, A. Shimoni, A. Nagler, J. Schachter, Adoptive Transfer of Tumor-Infiltrating Lymphocytes in Patients with Metastatic Melanoma: Intent-to-Treat Analysis and Efficacy after Failure to Prior Immunotherapies. Clinical Cancer Research 19, 4792-4800 (2013); A. J. Schoenfeld, S. M. Lee, B. Doger de Spéville, S. N. Gettinger, S. Häfliger, A. Sukari, S. Papa, J. F. Rodríguez-Moreno, F. Graf Finckenstein, R. Fiaz, M. Catlett, G. Chen, R. Qi, E. L. Masteller, V. Gontcharova, K. He, Lifileucel, an Autologous Tumor-Infiltrating Lymphocyte Monotherapy, in Patients with Advanced Non-Small Cell Lung Cancer Resistant to Immune Checkpoint Inhibitors. Cancer Discovery 14, 1389-1402 (2024)). These failures arise from three major factors: 1) limited starting tumor material and minimal pre-existing TILs; 2) heterogeneous distribution of TILs within a large number of tissue fragments that cannot be all processed under GMP; 3) variable functional quality of isolated TILs, including the presence of terminally exhausted CTLs that have lost proliferative capacity and effector function. Even when manufacturing is successful, the resulting TIL products often exhibit wide variability in cancer reactivity, leading to inconsistent clinical outcomes. Notably, TIL cultures from responding patients often establish more quickly than those from non-responders (M. J. Besser, R. Shapira-Frommer, O. Itzhaki, A. J. Treves, D. B. Zippel, D. Levy, A. Kubi, N. Shoshani, D. Zikich, Y. Ohayon, D. Ohayon, B. Shalmon, G. Markel, R. Yerushalmi, S. Apter, A. Ben-Nun, E. Ben-Ami, A. Shimoni, A. Nagler, J. Schachter, Adoptive Transfer of Tumor-Infiltrating Lymphocytes in Patients with Metastatic Melanoma: Intent-to-Treat Analysis and Efficacy after Failure to Prior Immunotherapies. Clinical Cancer Research 19, 4792-4800 (2013); M. J. Besser, R. Shapira-Frommer, A. J. Treves, D. Zippel, O. Itzhaki, L. Hershkovitz, D. Levy, A. Kubi, E. Hovav, N. Chermoshniuk, B. Shalmon, I. Hardan, R. Catane, G. Markel, S. Apter, A. Ben-Nun, I. Kuchuk, A. Shimoni, A. Nagler, J. Schachter, Clinical Responses in a Phase II Study Using Adoptive Transfer of Short-term Cultured Tumor Infiltration Lymphocytes in Metastatic Melanoma Patients. Clinical Cancer Research 16, 2646-2655 (2010)). Thus, identifying functional heterotypic CTLs that actively engage and kill tumor cells is critical for improving the success of TIL manufacturing and therapy (S. Ibáñez-Molero, J. Veldman, J. Simon Nieto, J. J. H. Traets, A. George, K. Hoefakker, A. Karomi, R. Harkes, B. van den Broek, S. M. Pack, L. Tas, N. L. Visser, S. E. van Hal-van Veen, P. Alóndiga-Mérida, M. Alkemade, I. M. Seignette, R. Tissier, M. Nieuwland, M. van Baalen, J. Poźniak, E. Mul, S. Tol, S. Stenqvist, L. M. Nilsson, J. A. Nilsson, J. Haanen, W. J. van Houdt, D. S. Peeper, Tumour-reactive heterotypic CD8 T cell clusters from clinical samples. Nature 649, 467-476 (2026)).

Current approaches for detecting functional TILs in surgical resections have significant limitations. Immunohistochemistry and flow cytometry are the most used methods for T cell assessments but are inherently destructive (cells are not manufacturable after analysis), time-consuming, labor-intensive, and use much of tumor samples. More recently, multiplexed digital PCR approaches have been applied to accelerate T cell detection. Nevertheless, measurement of genetic material does not necessarily reflect cytotoxic function. Moreover, these methods analyze only small aliquots, extrapolating across highly heterogeneous tumor tissues. Collectively, these limitations highlight a critical unmet need for a rapid, non-destructive, high-throughput approach capable of screening multiple intact tumor fragments simultaneously and identifying functional, cancer-reactive TILs while preserving their viability for manufacturing.

Granule-mediated cytotoxicity is a unique mechanism employed by CTLs to eliminate target cells. Upon antigen recognition, CTLs release cytotoxic granules containing perforin and granzyme B (GzmB), a potent serine protease that induces programmed cell death (S. P. Cullen, S. J. Martin, Mechanisms of granule-dependent killing. Cell Death Differ 15, 251-262 (2008)). GzmB imaging has been proposed as an effective means to predict CTL function.

The relationship between GzmB expression and CTL function, however, is highly state dependent. During T cell dysfunction, CTLs undergo a hierarchical differentiation from progenitor exhausted to terminally exhausted states (E. J. Wherry, M. Kurachi, Molecular and cellular insights into T cell exhaustion. Nature Review Immunology 15, 486-499 (2015)). Progenitor exhausted CTLs retain long-term persistence and polyfunctionality but express minimal GzmB and exhibit little cytotoxic activity beyond that of naïve CD8+ T cells (B. C. Miller, D. R. Sen, R. Al Abosy, K. Bi, Y. V. Virkud, M. W. LaFleur, K. B. Yates, A. Lako, K. Felt, G. S. Naik, M. Manos, E. Gjini, J. R. Kuchroo, J. J. Ishizuka, J. L. Collier, G. K. Griffin, S. Maleri, D. E. Comstock, S. A. Weiss, F. D. Brown, A. Panda, M. D. Zimmer, R. T. Manguso, F. S. Hodi, S. J. Rodig, A. H. Sharpe, W. N. Haining, Subsets of exhausted CD8(+) T cells differentially mediate tumor control and respond to checkpoint blockade. Nat Immunol 20, 326-336 (2019)). In contrast, terminally exhausted CTLs express high levels of GzmB (B. C. Miller, D. R. Sen, R. Al Abosy, K. Bi, Y. V. Virkud, M. W. LaFleur, K. B. Yates, A. Lako, K. Felt, G. S. Naik, M. Manos, E. Gjini, J. R. Kuchroo, J. J. Ishizuka, J. L. Collier, G. K. Griffin, S. Maleri, D. E. Comstock, S. A. Weiss, F. D. Brown, A. Panda, M. D. Zimmer, R. T. Manguso, F. S. Hodi, S. J. Rodig, A. H. Sharpe, W. N. Haining, Subsets of exhausted CD8(+) T cells differentially mediate tumor control and respond to checkpoint blockade. Nat Immunol 20, 326-336 (2019)), yet are profoundly impaired in degranulation, target cell killing, and proliferative capacity (J. C. Riches, J. K. Davies, F. Mcclanahan, R. Fatah, S. Iqbal, S. Agrawal, A. G. Ramsay, J. G. Gribben, T cells from CLL patients exhibit features of T-cell exhaustion but retain capacity for cytokine production. Blood 121, 1612-1621 (2013); X. Wu, H. Zhang, Q. Xing, J. Cui, J. Li, Y. Li, Y. Tan, S. Wang, PD-1(+) CD8(+) T cells are exhausted in tumours and functional in draining lymph nodes of colorectal cancer patients. Br J Cancer 111, 1391-1399 (2014)). GzmB is intrinsically stable and remains catalytically active even within the acidic environment of cytotoxic granules prior to release (D. B.T., Dissertation, Ludwig Maximilian University of Munich, Munich (2010)). As a result, existing approaches that detect total GzmB protein or bulk enzymatic activity cannot discriminate between actively degranulating effector CTLs and terminally exhausted CTLs (S. He, J. Li, Y. Lyu, J. Huang, K. Pu, Near-Infrared Fluorescent Macromolecular Reporters for Real-Time Imaging and Urinalysis of Cancer Immunotherapy. Journal of the American Chemical Society 142, 7075-7082 (2020); J. I. Scott, S. Gutkin, O. Green, E. J. Thompson, T. Kitamura, D. Shabat, M. Vendrell, A Functional Chemiluminescent Probe for in Vivo Imaging of Natural Killer Cell Activity Against Tumours. Angewandte Chemie, International Edition 60, 5699-5703 (2021); J. Xie, F. El Rami, K. Zhou, F. Simonetta, Z. Chen, X. Zheng, M. Chen, P. B. Balakrishnan, S. Y. Dai, S. Murty, I. S. Alam, J. Baker, R. S. Negrin, S. S. Gambhir, J. Rao, Multiparameter Longitudinal Imaging of Immune Cell Activity in Chimeric Antigen Receptor T Cell and Checkpoint Blockade Therapies. ACS Central Science 8, 590-602 (2022); J. I. Scott, L. Mendive-Tapia, D. Gordon, N. D. Barth, E. J. Thompson, Z. Cheng, D. Taggart, T. Kitamura, A. Bravo-Blas, E. W. Roberts, J. Juarez-Jimenez, J. Michel, B. Piet, I. J. de Vries, M. Verdoes, J. Dawson, N. O. Carragher, R. A. O. Connor, A. R. Akram, M. Frame, A. Serrels, M. Vendrell, A fluorogenic probe for granzyme B enables in-biopsy evaluation and screening of response to anticancer immunotherapies. Nature Communications 13, 2366 (2022); L. Xu, N. Liu, W. Zhan, Y. Deng, Z. Chen, X. Liu, G. Gao, Q. Chen, Z. Liu, G. Liang, Granzyme B Turns Nanoparticle Fluorescence “On” for Imaging Cytotoxic T Lymphocyte Activity in Vivo. ACS Nano 16, 19328-19334 (2022)). More importantly, because TILs isolated from tumor resections constitute a living therapeutic product, it is undesirable for imaging probes to directly interact with or perturb T cells, as this may compromise their viability or suitability for downstream GMP expansion (N. Zhao, C. Bardine, A. L. Lourenço, Y. H. Wang, Y. Huang, S. J. Cleary, D. M. Wilson, D. Y. Oh, L. Fong, M. R. Looney, M. J. Evans, C. S. Craik, In Vivo Measurement of Granzyme Proteolysis from Activated Immune Cells with PET. ACS Central Science 7, 1638-1649 (2021)). Accordingly, a functional imaging strategy that selectively reports the activity of degranulated GzmB at sites of tumor cell killing should more faithfully reflect TIL cytotoxic function without compromising cell viability.

To realize this new strategy, AGB-CyP was designed and utilized, which couples an anti-GzmB single-chain variable fragment (scFv) (AGB, ~28 kDa) to an IEPD (Ile-Glu-Pro-Asp; SEQ ID NO:8) peptide-caged near-infrared hemicyanine fluorochrome (CyP) (22). The scFv antibody confers high-affinity binding and in situ probe retention without interrupting the catalytic activity of GzmB. The IEPD sequence (SEQ ID NO:8) serves as a selective substrate for human GzmB (L. Yuan, W. Lin, S. Zhao, W. Gao, B. Chen, L. He, S. Zhu, A unique approach to development of near-infrared fluorescent sensors for in vivo imaging. J Am Chem Soc 134, 13510-13523 (2012)), ensuring that fluorescence activation occurs exclusively upon proteolytic cleavage by GzmB. The near-infrared hemicyanine fluorochrome was chosen to support deep penetration and high contrast imaging in intact tissues. Owing to its macromolecular size, AGB-CyP cannot freely diffuse across intact T cell membranes, thereby eliminating background signal from intracellular GzmB retained within terminally exhausted CTLs. Instead, the probe is designed to access tumor cells through perforin pores formed during CTL-mediated attack, allowing sensitive detection of GzmB both in the extracellular tumor microenvironment and within engaged cancer cells. This spatially restricted activation maximizes detection sensitivity while maintaining strict functional specificity. By remaining completely non-permeable to T cells, AGB-CyP preserves TIL viability and compatibility with downstream GMP expansion. Together, these design principles enable AGB-CyP to provide a real-time, non-destructive functional readout of CTL activity that directly distinguishes tumor-killing effector CTLs from exhausted populations.

### C. Results

### Granzyme B Localizes to CTL-Engaged Tumor Cells in Human Melanoma Specimens

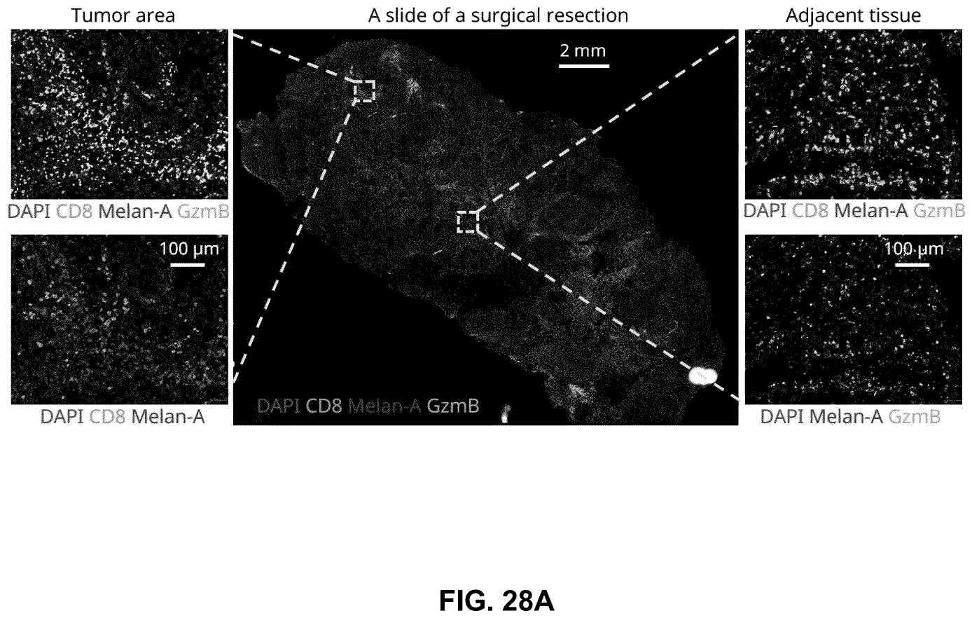

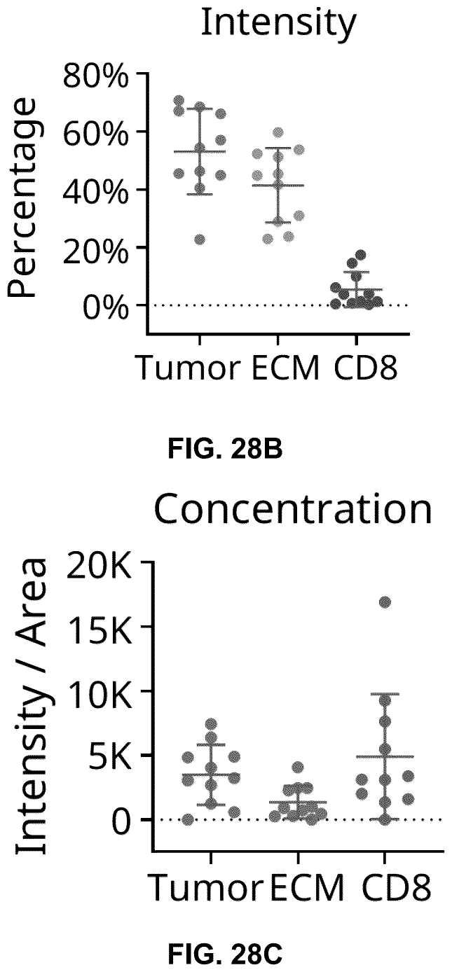

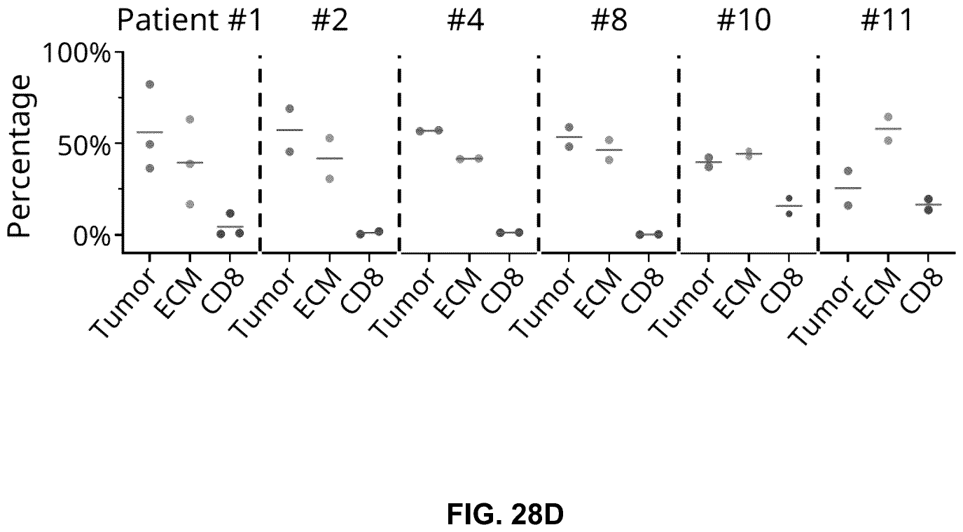

To determine the spatial distribution of granzyme B (GzmB) in human tumors, melanoma resections from 11 patients were analyzed, including both pediatric and adult cases. Multiplex immunofluorescence staining for melanoma cells (Melan-A, cytosolic), cytotoxic T lymphocytes (CTLs) (CD8a), GzmB, and nuclei (DAPI) revealed heterogeneous CTLs infiltration across tumor and adjacent non-malignant tissues (FIG. 28A). Many of the CTLs in non-malignant tissues were stained positive for intracellular GzmB, underscoring the spatial heterogeneity of CTLs within surgical specimens and the challenge of selecting optimal fragments for TIL manufacturing. Within tumor regions, GzmB signal strongly colocalized with the Melan-A positive melanoma cells (FIG. 28A, tumor area). Quantitative fluorescence analysis across all 11 patients demonstrated that over 50% of total GzmB signals on average were associated with tumor cells, whereas a smaller fraction remained extracellular (FIG. 28b). GzmB is highly enriched within CTLs and CTL-engaged tumor cells (FIG. 28C). In patients with multiple tumor lesions, GzmB distribution patterns were consistent across independent tumors (FIG. 28D), suggesting reproducibility of this spatial phenomenon. Together, these findings indicate that functionally released GzmB accumulates within tumor cells engaged by CTLs. Therefore, a detection strategy optimized to capture catalytic GzmB activity both intracellularly within target tumor cells and extracellularly in the tumor microenvironment is essential for maximizing detection sensitivity.

### Design and Generation of AGB-CyP, a Fluorogenic Probe for Imaging Degranulated Granzyme B

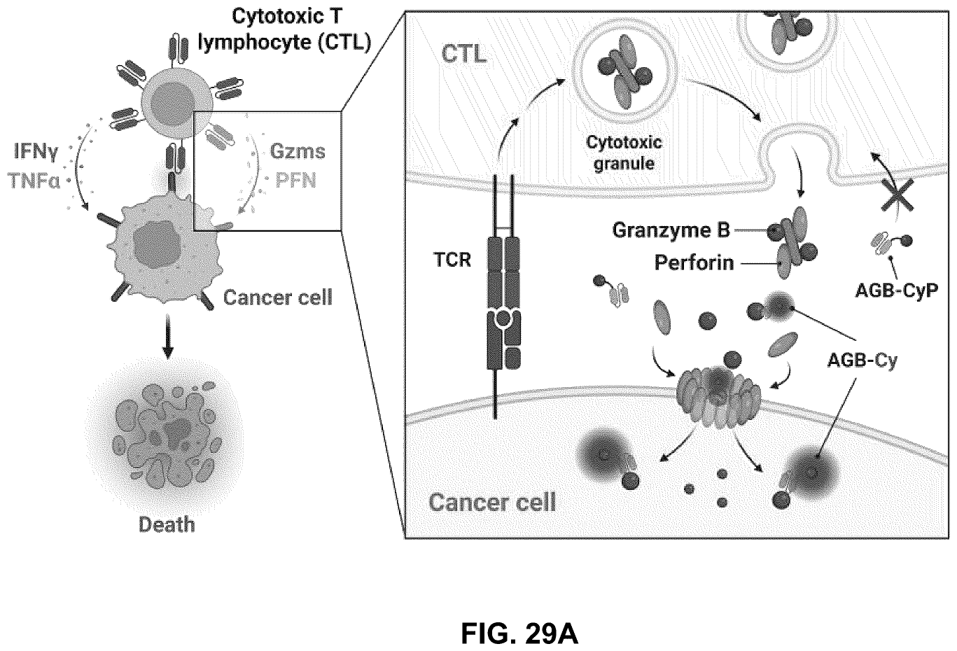

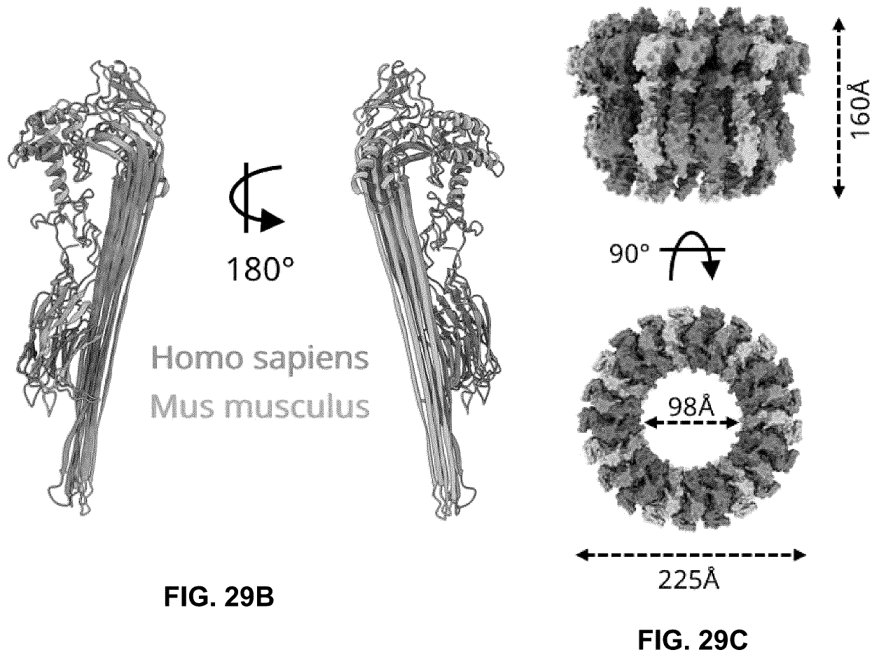

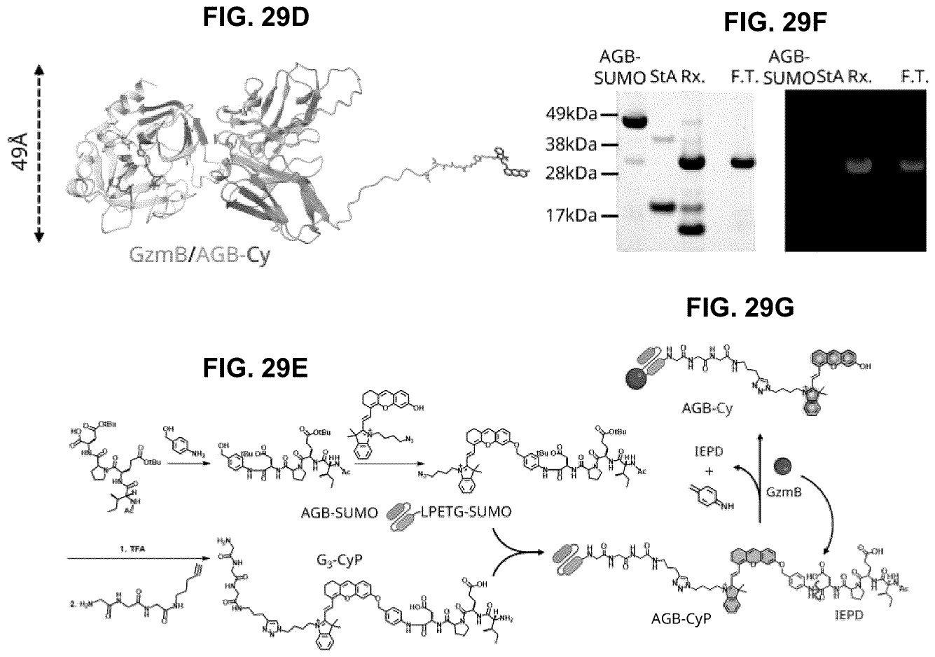

AGB-CyP was engineered, a near-infrared fluorogenic single-chain antibody probe designed to activate upon cleavage by degranulated GzmB during CTL-mediated cytotoxicity (FIG. 29A). Upon activation, fluorescent AGB-Cy probe remains localized through high-affinity binding to GzmB, enabling signal retention at sites of immune attack, both extracellularly and within CTL-engaged tumor cells. Because entry into tumor cells requires passage through perforin pores, it was evaluated whether AGB-CyP could physically traverse the assembled perforin pore. Using AlphaFold and structural guidance from a murine cryo-EM perforin pore model (24), the structure of human perforin monomer was predicted and simulated an assembled perforin pore complex. Although human and mouse perforin monomers share only 68% sequence identities, the predicted secondary structures are highly comparable, including the long hydrophobic β-sheets essential for transmembrane pore formation (FIG. 29B). When solubilized murine perforin pores were determined, the structures showed symmetry ranging from C15 to C26, with most having 21- to 23-fold symmetry (M. E. Ivanova, N. Lukoyanova, S. Malhotra, M. Topf, J. A. Trapani, I. Voskoboinik, H. R. Saibil, The pore conformation of lymphocyte perforin. Sci Adv 8, eabk3147 (2022)). Therefore, a C21 molecular model of human was stimulated (FIG. 29C). This modeled pore exhibited an external diameter of ~225 Å and a height of ~160 Å, with a minimal internal opening of ~98 Å. This dimension is sufficient to accommodate a GzmB monomer (~27 kDa), granzyme A dimer (~60 kDa), and AGB-CyP (~28 kDa), supporting the feasibility of probe entry into perforin-permeabilized tumor cells. Structural modeling further predicted that AGB-Cy binding occurs distal to the catalytic pocket of GzmB (Rotonda, M. Garcia-Calvo, H. G. Bull, W. M. Geissler, B. M. Mckeever, C. A. Willoughby, N. A. Thornberry, J. W. Becker, The three-dimensional structure of human granzyme B compared to caspase-3, key mediators of cell death with cleavage specificity for aspartic acid in P1. Chem Biol 8, 357-368 (2001)), minimizing interference with enzymatic activity (FIG. 29D).

AGB-CyP was generated via sortase A-mediated bio-ligation (C. P. Guimaraes, M. D. Witte, C. S. Theile, G. Bozkurt, L. Kundrat, A. E. M. Blom, H. L. Ploegh, Site-specific C-terminal and internal loop labeling of proteins using sortase-mediated reactions. Nature Protocols 8, 1787-1799 (2013)), linking chemically synthesized triglycine-CyP (G3-CyP) compound to recombinant AGB (FIG. 29E). Fluorogenic CyP molecule was synthesized following an established protocols (S. He, J. Li, Y. Lyu, J. Huang, K. Pu, Near-Infrared Fluorescent Macromolecular Reporters for Real-Time Imaging and Urinalysis of Cancer Immunotherapy. Journal of the American Chemical Society 142, 7075-7082 (2020)). Briefly, Ac-IEPD-OH (SEQ ID NO: 9) was first coupled to 4-aminobenzyl alcohol. The coupling product was then brominated and subsequently reacted with the hydroxyl group of the hemicyanine to generate CyP. The OtBu (tert-butyl ester) group on CyP was then removed by using TFA (trifluoroacetic acid), and the deprotected product reacted with triglycine-alkyne by copper (I) catalyzed click reaction to generate the product G₃-CyP. A SUMO-6His tag downstream of the LPETG sortase A recognition motif allowed visualization of ligation efficiency on SDS-PAGE (FIG. 29F). Following ligation, SUMO-6His and sortase A were removed by affinity purification. The resulting AGB-CyP probe enables in situ near-infrared fluorescence activation upon GzmB cleavage and signal retention via high-affinity binding (FIG. 29G). Importantly, probe activation and binding did not impair GzmB catalytic activity, establishing AGB-CyP as an orthogonal, non-inhibitory reporter of GzmB degranulation.

### AGB-CyP Specifically Reports Granzyme B-Mediated T Cell Cytotoxicity

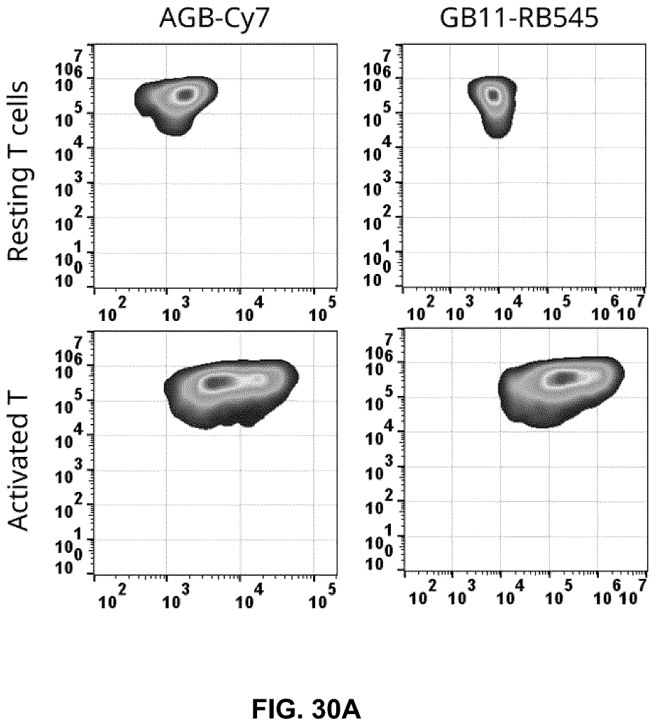

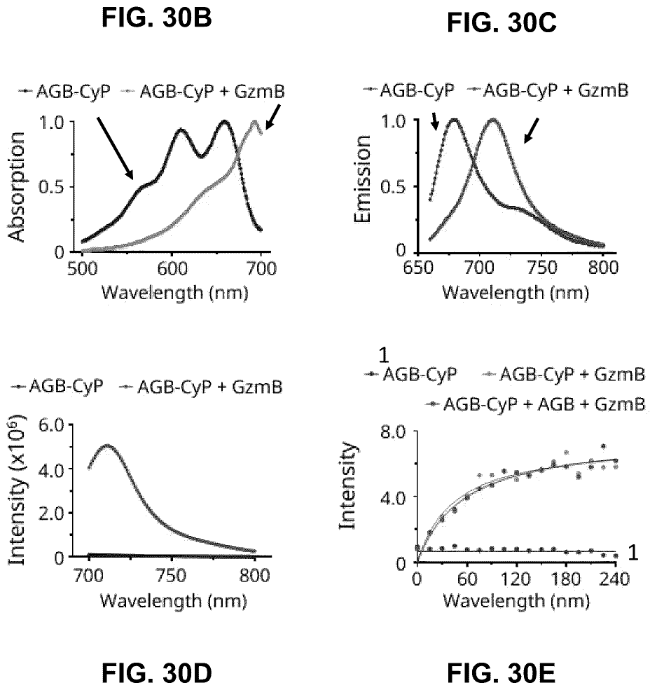

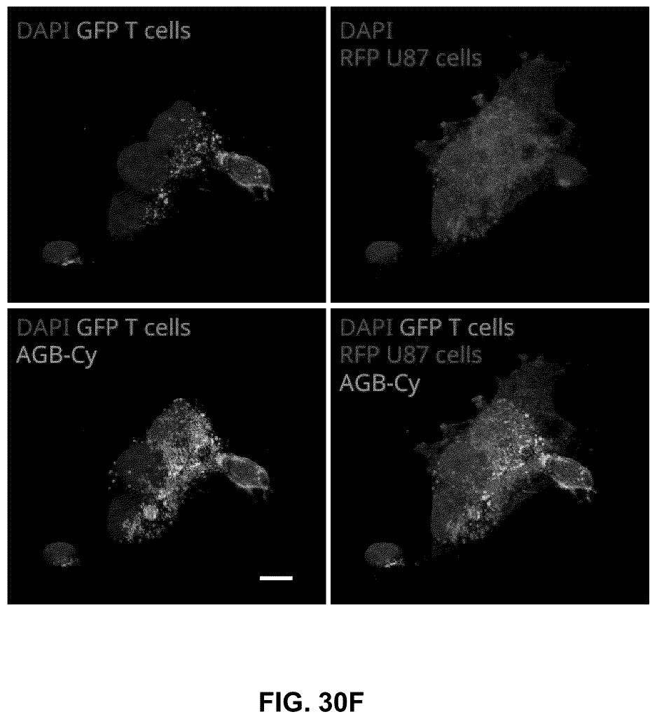

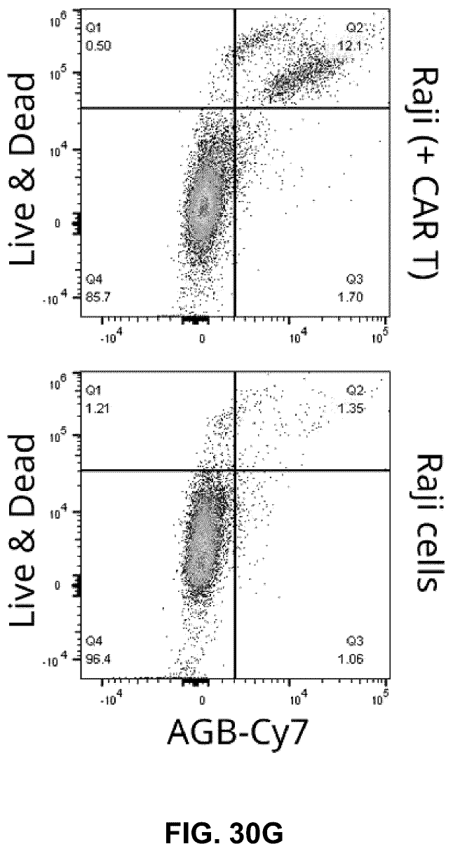

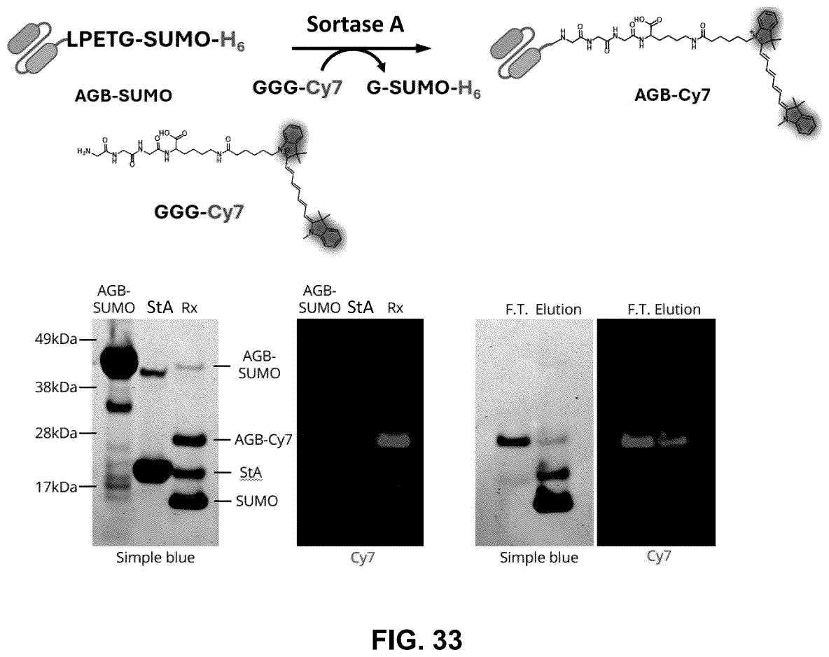

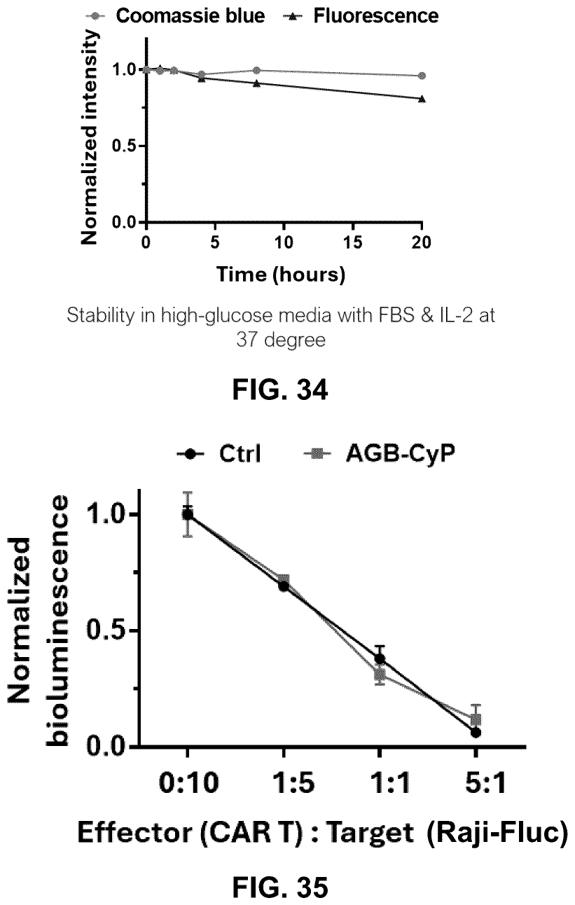

To confirm binding specificity of AGB to human GzmB, an AGB-Cy7 variant was generated (FIG. 33) suitable for flow cytometry. AGB-Cy7 staining of resting and CD3/CD28 beads-activated CD8+ T cells showed distributions comparable to a commercial anti-GzmB antibody conjugated to RB545 (FIG. 30A), indicating high specificity. Optically, AGB-CyP exhibits favorable properties for intact tissue imaging. Prior to activation, the probe displays dual excitation peaks at 610 nm and 660 nm with weak emission at 675 nm (FIGS. 30B-3C). Upon GzmB cleavage, fluorescence undergoes a red shift to excitation at 690 nm and emission at 715 nm, resulting in approximately 60-fold signal enhancement (FIG. 30B and FIG. 30D). The probe remained stable in T cell culture media (FIG. 34). Pre-incubation of GzmB with AGB did not alter catalytic activation kinetics (FIG. 30E), consistent with structural predictions.

To validate functional specificity, GFP-expressing CD19-28ζ CAR T cells were co-cultured with RFP-expressing CD19⁺ U87 glioblastoma cells in the presence of AGB-CyP. Fluorescence was observed exclusively within tumor cells engaged by CAR T cells (FIG. 30F and movie S1, movie not shown), demonstrating selective detection of released GzmB and probe permeability through perforin pores during cytotoxic killing. Importantly, incubation with AGB-CyP has no impact on the cytotoxic function of CAR T cells (FIG. 35). Flow cytometric analysis using AGB-Cy7 further confirmed probe retention in CAR T cell-killed Raji lymphoma cells (FIG. 30G). Collectively, these studies demonstrate that AGB-CyP selectively reports released GzmB from functional T cells during tumor cell killing, without detecting intracellular GzmB retained in non-degranulating cells.

### Establishment of a Lymphoma Spheroid CAR T Cell Model to Study T Cell Function

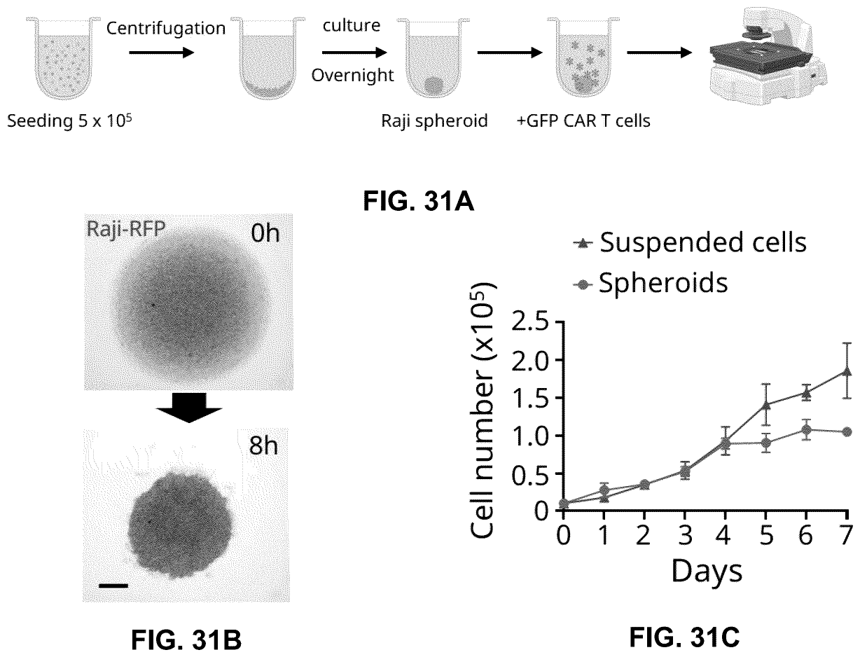

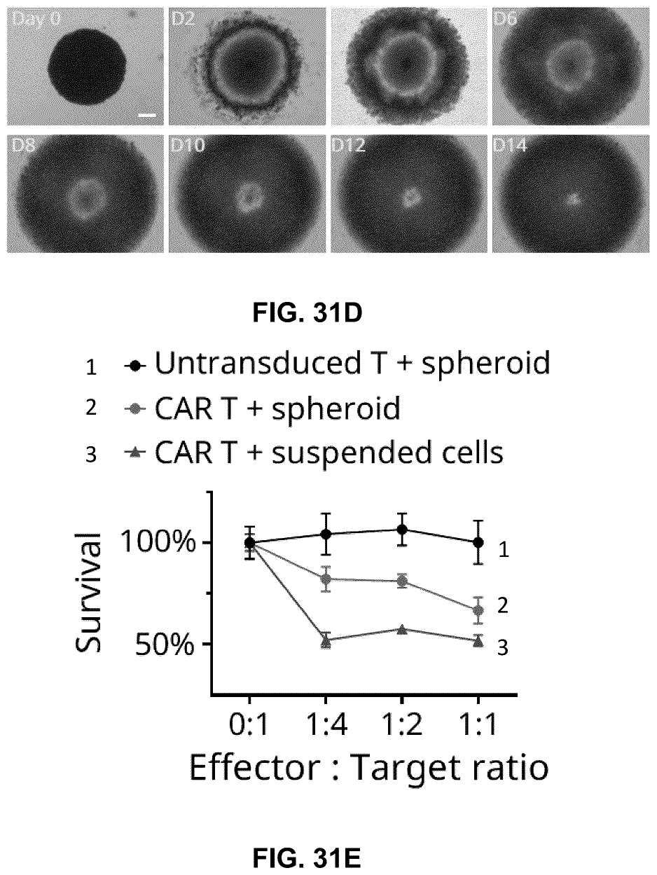

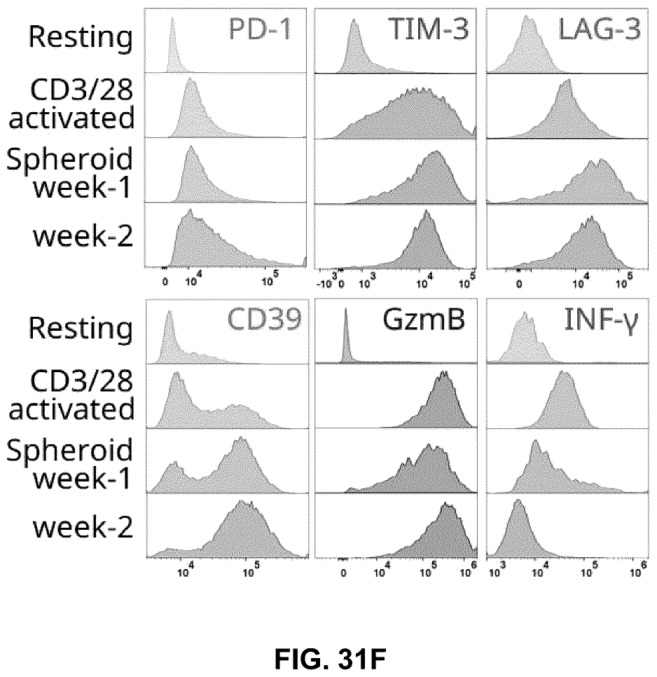

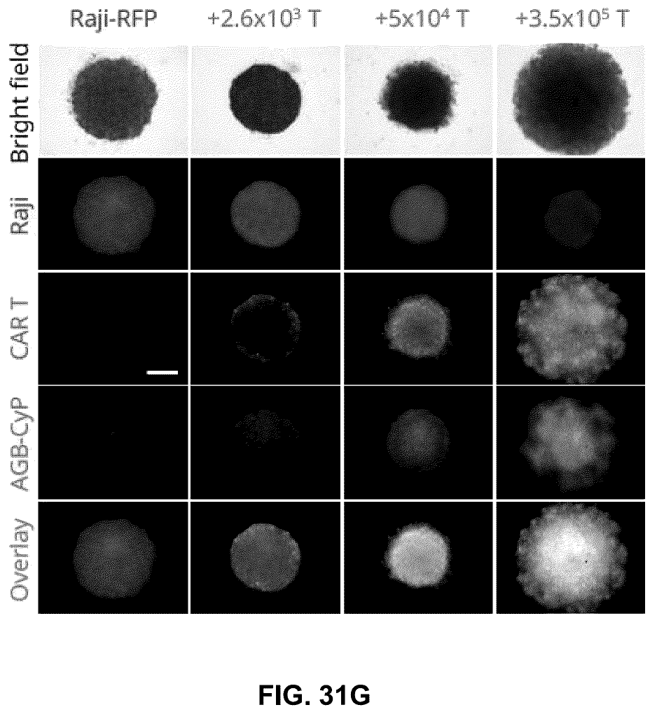

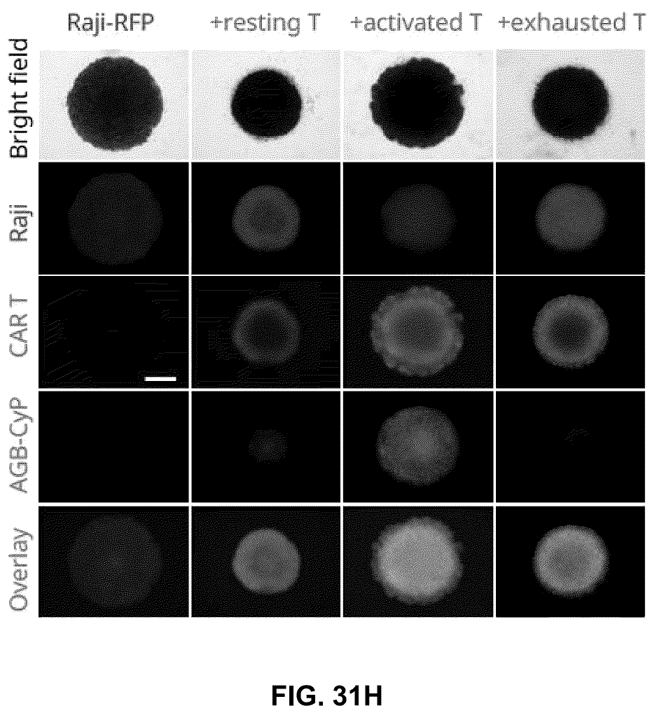

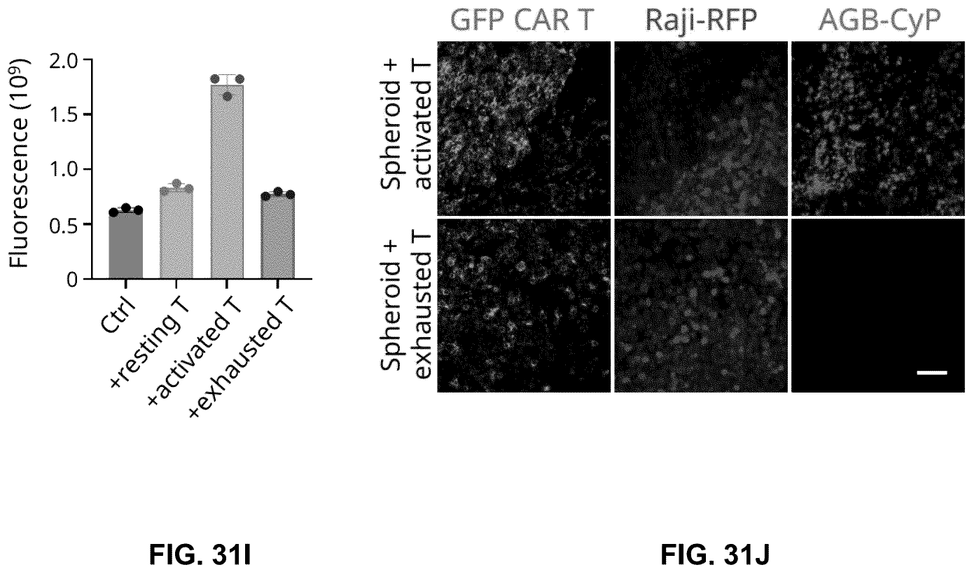

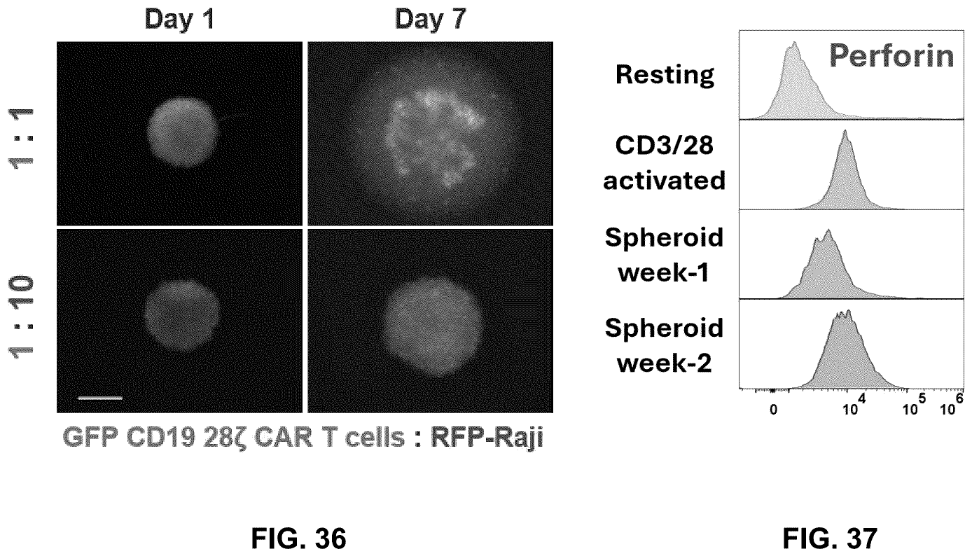

To evaluate probe performance within a three-dimensional tumor microenvironment, floating lymphoma spheroids were established using Raji cells and Raji variants expressing RFP or firefly luciferase (Fluc) (FIG. 31A). Uniform spheroids formed within 8 hours from 5×10⁵ cells in U-bottom plates (FIG. 31A). Compared to suspension culture, spheroid growth plateaued after reaching ~900 nm in diameter by day 4, indicating nutrient and oxygen limitations (FIG. 31C and movie S3, movie not shown). At a 1:1 effector-to-target ratio, GFP-CAR T cells efficiently infiltrated and eradicated spheroids within two weeks, albeit more slowly than in suspension culture (FIG. 31D and FIG. 31E, and movie S3, not shown). When the ratio was reduced to 1:10, CAR T cells progressively lost tumor control (FIG. 36) and developed a terminally exhausted phenotype characterized by high PD-1, TIM-3, LAG-3, and CD39 expression (FIG. 31F). Exhausted CAR T cells retained high intracellular GzmB and perforin but exhibited reduced IFN-γ production relative to beads-activated T cells (FIG. 31F and FIG. 37). This spheroid system therefore recapitulates key aspects of T cell activation and dysfunction and provides a controlled platform for evaluating functional imaging probes.

AGB-CyP Sensitively Detects Functional T Cells and Distinguishes them from Terminally Exhausted T Cells in Tumor Spheroids

Raji-RFP spheroids were treated with 10², 10³ or 10⁴ resting GFP-CAR T cells. After 96 hours, spheroids were incubated with AGB-CyP for 2 hours, imaged, and subsequently dissociated to quantify infiltrating T cells. Spheroids initially seeded with 10² CAR T cells expanded to approximately 2.6×10³ T cells and produced detectable AGB-CyP signal (FIG. 31G), indicating high sensitivity of the probe in a 3D tumor context. To assess the ability of AGB-CyP in reporting T cell function, 5×10⁴ terminally exhausted CAR T cells were sorted and applied to spheroids (5×10⁵). Although terminally exhausted T cells infiltrated tumor spheroids comparably to freshly activated T cells, AGB-CyP signal was minimal (FIG. 31H and FIG. 31I). Confocal imaging confirmed retention of activated probe only in spheroids treated with activated functional T cells (FIG. 31J). Together, these data demonstrate that AGB-CyP sensitively detects 10³-10⁴ functional T cells within tumor spheroids and robustly discriminates functional CTLs from terminally exhausted T cells that retain intracellular GzmB but fail to degranulate.

### Detection of Granzyme B in Surgical Tumor Resections

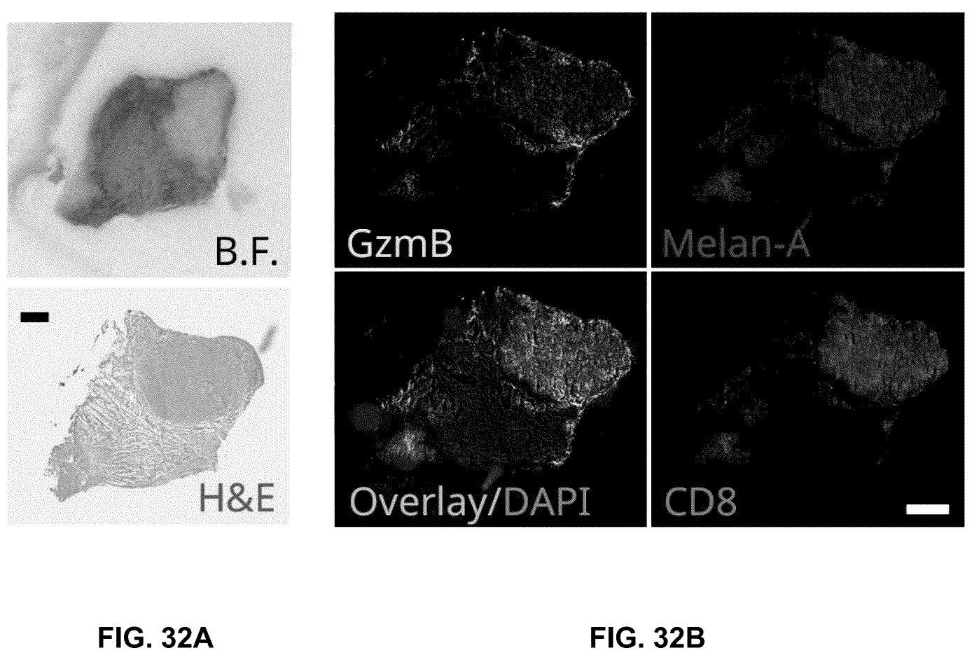

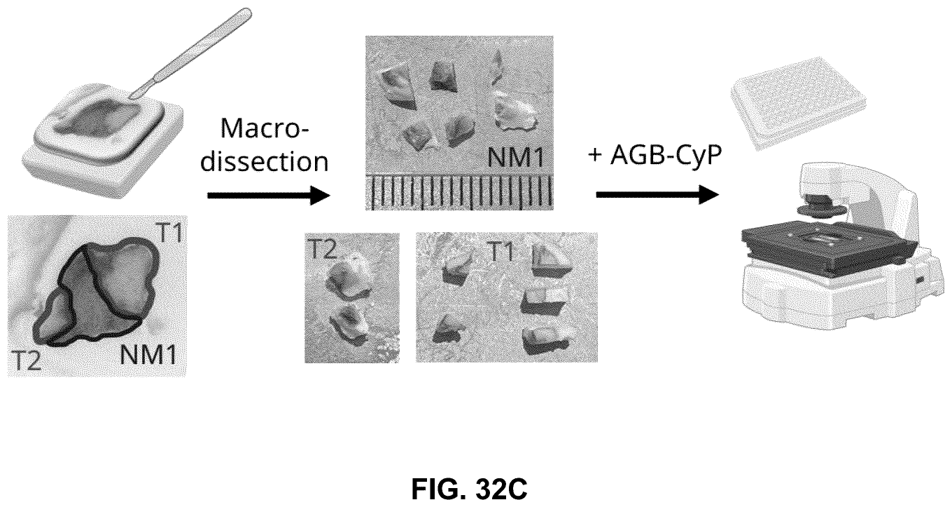

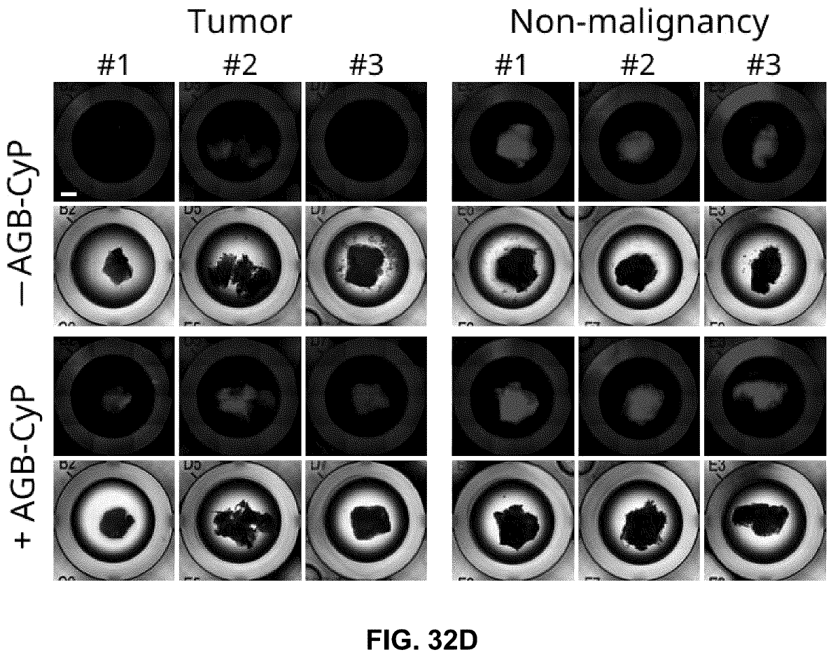

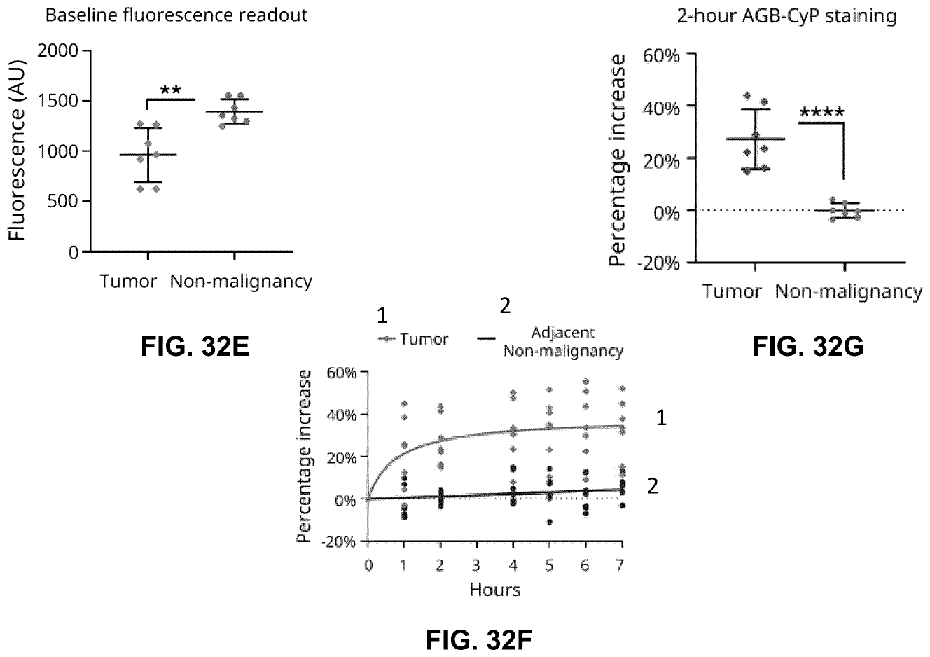

To assess the feasibility of applying AGB-CyP to clinical specimens, its performance was evaluated in an archived fresh-frozen melanoma surgical resection (~0.65 g) containing both tumor and adjacent non-malignant tissue (FIG. 32A, FIG. 32C). Tumor regions, defined by Melan-A positivity, exhibited dense TIL infiltration and abundant GzmB staining (FIG. 32B). Because the specimen had been snap-frozen in liquid nitrogen, cellular membrane integrity, particularly of TILs, may have been partially compromised. Thus, this experiment was designed primarily to determine whether AGB-CyP could detect catalytically active GzmB and retain signal in situ independent of intact T cell membranes. Notably, GzmB is known to remain enzymatically active after freezing, allowing functional assessment of its catalytic activity despite cellular architecture. Using imaging-guided macrodissection, replicate fragments from tumor regions (T1, T2) and adjacent non-malignant tissue (NM1) were generated. Fragments were incubated with AGB-CyP in a 96-well plate and longitudinally imaged using an automated fluorescence microscopy scanner. At baseline (prior to probe incubation), non-malignant tissue fragments exhibited higher intrinsic fluorescence than tumor fragments (FIG. 32D and FIG. 32E), likely reflecting greater structural heterogeneity and autofluorescent stromal components in normal tissue. Despite this baseline, tumor fragments demonstrated a progressive and preferential increase in near-infrared fluorescence following probe incubation (FIG. 32F). Fluorescence enhancement in tumor tissues became detectable within 2 hours (FIG. 32G). Importantly, non-malignant fragments showed minimal longitudinal fluorescence increase, indicating limited catalytic activation outside tumor regions (FIG. 32B). This selective signal amplification within tumor tissue supports the specificity of AGB-CyP for detecting catalytically active GzmB in human surgical specimens. Collectively, these data prove that AGB-CyP can generate spatially retained, function-dependent fluorescence in intact tissues. This proof-of-concept experiment establishes the feasibility of applying AGB-CyP to surgical tumor specimens and supports further development for rapid functional evaluation of TILs for improved manufacturing and therapy.

### D. Discussion

Adoptive transfer of tumor-infiltrating lymphocytes (TILs) has emerged as a transformative therapy for advanced solid tumors, yet its broader clinical impact is constrained by a fundamental limitation: the absence of a rapid, non-destructive method to determine whether resected tumor biospecimens contain functional, cancer-reactive cytotoxic T lymphocytes (CTLs) prior to manufacturing. In the present example, AGB-CyP is introduced, a near-infrared fluorogenic scFv probe that selectively reports degranulated granzyme B (GzmB) activity, and its ability to enable functional imaging of TIL activity directly within intact surgical tumor resections demonstrated. Together, these data establish a conceptual and technical framework for real-time, biospecimen-level functional immune assessment to guide TIL manufacturing.

Current approaches to evaluate TILs—such as immunohistochemistry, flow cytometry, or nucleic acid-based assays—primarily quantify cell presence or gene expression rather than cytotoxic function. These methods are inherently destructive, limited to small aliquots, and poorly suited to address the spatial heterogeneity of large tumor resections. Importantly, expression of phenotypic markers, including CD8 or even GzmB protein, does not necessarily reflect active tumor killing, particularly in the context of T cell exhaustion.

Immunofluorescence analysis of melanoma resections from 11 patients revealed that more than half of detectable GzmB signal localized within Melan-A⁺ tumor cells, with a smaller fraction distributed extracellularly. This spatial enrichment supports a model in which functionally released GzmB accumulates preferentially within CTL-engaged tumor cells. These findings provide human tissue—level evidence that degranulation, rather than total intracellular GzmB content, more faithfully reflects active cytotoxic engagement. By engineering AGB-CyP to detect catalytically active, released GzmB—while excluding intracellular GzmB retained in intact T cells—we shift the focus from static immune profiling to dynamic functional imaging of tumor cell killing.

AGB-CyP was rationally designed to satisfy several constraints essential for a clinically compatible TIL screening assay. First, the probe must discriminate functional CTLs from terminally exhausted T cells, which often retain high intracellular GzmB levels yet exhibit impaired degranulation and proliferative capacity (REF). Second, it must preserve T cell viability and compatibility with downstream GMP expansion. Third, it must provide sufficient sensitivity to detect relatively rare functional T cells within heterogeneous tissue fragments.

Structural modeling predicted that the ~28 kDa scFv-based probe can traverse perforin pores, whose minimal internal diameter (~98 Å) is sufficient to accommodate AGB-CyP and the AGB-Cy/GzmB complex. This pore-permissive but T cell-impermeable design allows the probe to access GzmB in perforin-permeabilized tumor cells while preventing nonspecific entry into intact T cells. In two-dimensional co-culture and three-dimensional lymphoma spheroid models, AGB-CyP selectively activated in tumor cells engaged by functional CAR T cells but remained largely silent in spheroids infiltrated by terminally exhausted T cells, despite their high intracellular GzmB content. These data validate that degranulation-dependent activation provides a stringent functional filter that distinguishes tumor-killing CTLs from dysfunctional populations.

Notably, AGB-CyP achieved detection of approximately 10³-10⁴ functional T cells within 3D spheroids, highlighting its sensitivity in tissue-like contexts. By coupling enzymatic activation with target binding, AGB-CyP integrates both activity and localization, enabling spatially retained signal at sites of immune attack.

Application of AGB-CyP to fresh-frozen melanoma resections demonstrated progressive, tumor-selective near-infrared signal enhancement within two hours of incubation. Although baseline autofluorescence was higher in adjacent non-malignant tissue, only tumor fragments exhibited substantial longitudinal fluorescence increases, consistent with the presence of catalytically active GzmB. Because these samples had undergone snap-freezing, membrane integrity—particularly of T cells—was likely partially compromised. Thus, this experiment primarily established that catalytically active GzmB can be detected and spatially retained in intact tissue fragments.

Importantly, this example supports the feasibility of a rapid, whole-fragment assay format compatible with the early stages of TIL manufacturing. Unlike destructive immunophenotyping approaches, AGB-CyP imaging can be performed directly on intact tumor fragments prior to enzymatic digestion, preserving viable cells for expansion. In principle, such a strategy could allow prioritization of fragments enriched for functional, cancer-reactive CTLs, potentially reducing manufacturing failures and improving therapeutic consistency.

The clinical success of TIL therapy remains limited by high manufacturing failure rates and variability in product potency. Even when expansion succeeds, the functional quality of TIL products varies substantially, contributing to heterogeneous clinical responses. A non-destructive, functional prescreening assay could address multiple bottlenecks: (i) identifying patients whose tumors harbor functional CTLs, (ii) selecting optimal fragments for rapid expansion, and (iii) reducing time and cost associated with unsuccessful manufacturing attempts.

Beyond TIL therapy, this strategy may have broader applications in adoptive cell therapies and immuno-oncology. Functional imaging of degranulation could inform evaluation of CAR T cell potency in solid tumors, assessment of tumor immune competence in ex vivo biopsies, and early prediction of response to immunotherapies (REF). Because AGB-CyP reports a fundamental effector mechanism—granule-mediated cytotoxicity—it provides a modality-agnostic readout applicable across diverse T cell-based interventions.

In summary, described herein is a near-infrared fluorogenic scFv probe that selectively reports degranulated granzyme B activity and enables rapid, non-destructive functional imaging of tumor-reactive CTLs in intact surgical resections. By directly visualizing cytotoxic activity rather than inferring function from static markers, AGB-CyP provides a biologically grounded and clinically actionable readout of TIL potency. This example establishes the foundation for a companion biospecimen assay to streamline TIL manufacturing and improve patient selection, and it opens new avenues for functional immune profiling in cancer

### E. References Related to the Present Example

1. S. A. Rosenberg, P. Spiess, R. Lafreniere, A new approach to the adoptive immunotherapy of cancer with tumor-infiltrating lymphocytes. Science 233, 1318-1321 (1986).
2. A. Betof Warner, P. G. Corrie, O. Hamid, Tumor-Infiltrating Lymphocyte Therapy in Melanoma: Facts to the Future. Clinical Cancer Research 29, 1835-1854 (2023).
3. F. Crossey, S. Marx, S. Holters, K. Schmitt, R. M. Bohle, T. Schmidt, M. Stockle, U. Sester, M. Sester, M. W. W. Janssen, Robust method for isolation of tumor infiltrating lymphocytes with a high vital cell yield from small samples of renal cell carcinomas by a new collagenase-free mechanical procedure. Urologic Oncology 36, 402.e401-402.e410 (2018).
4. O. J. Fulbright, M.-A. Forget, C. Haymaker, C. Bernatchez, in Cancer Immunoprevention: Methods and Protocols, F. McAllister, Ed. (Springer US, New York, NY, 2022), pp. 43-71.
5. R. Andersen, T. H. Borch, A. Draghi, A. Gokuldass, M. A. H. Rana, M. Pedersen, M. Nielsen, P. Kongsted, J. W. Kjeldsen, M. C. W. Westergaard, H. D. Radic, C. A. Chamberlain, L. R. Holmich, H. W. Hendel, M. S. Larsen, O. Met, I. M. Svane, M. Donia, T cells isolated from patients with checkpoint inhibitor-resistant melanoma are functional and can mediate tumor regression. Annals of Oncology 29, 1575-1581 (2018).
6. M. J. Besser, R. Shapira-Frommer, O. Itzhaki, A. J. Treves, D. B. Zippel, D. Levy, A. Kubi, N. Shoshani, D. Zikich, Y. Ohayon, D. Ohayon, B. Shalmon, G. Markel, R. Yerushalmi, S. Apter, A. Ben-Nun, E. Ben-Ami, A. Shimoni, A. Nagler, J. Schachter, Adoptive Transfer of Tumor-Infiltrating Lymphocytes in Patients with Metastatic Melanoma: Intent-to-Treat Analysis and Efficacy after Failure to Prior Immunotherapies. Clinical Cancer Research 19, 4792-4800 (2013).
7. A. J. Schoenfeld, S. M. Lee, B. Doger de Spéville, S. N. Gettinger, S. Häfliger, A. Sukari, S. Papa, J. F. Rodríguez-Moreno, F. Graf Finckenstein, R. Fiaz, M. Catlett, G. Chen, R. Qi, E. L. Masteller, V. Gontcharova, K. He, Lifileucel, an Autologous Tumor-Infiltrating Lymphocyte Monotherapy, in Patients with Advanced Non-Small Cell Lung Cancer Resistant to Immune Checkpoint Inhibitors. Cancer Discovery 14, 1389-1402 (2024).
8. M. J. Besser, R. Shapira-Frommer, A. J. Treves, D. Zippel, O. Itzhaki, L. Hershkovitz, D. Levy, A. Kubi, E. Hovav, N. Chermoshniuk, B. Shalmon, I. Hardan, R. Catane, G. Markel, S. Apter, A. Ben-Nun, I. Kuchuk, A. Shimoni, A. Nagler, J. Schachter, Clinical Responses in a Phase II Study Using Adoptive Transfer of Short-term Cultured Tumor Infiltration Lymphocytes in Metastatic Melanoma Patients. Clinical Cancer Research 16, 2646-2655 (2010).
9. S. Ibáñez-Molero, J. Veldman, J. Simon Nieto, J. J. H. Traets, A. George, K. Hoefakker, A. Karomi, R. Harkes, B. van den Broek, S. M. Pack, L. Tas, N. L. Visser, S. E. van Hal-van Veen, P. Alóndiga-Merida, M. Alkemade, I. M. Seignette, R. Tissier, M. Nieuwland, M. van Baalen, J. Poźniak, E. Mul, S. Tol, S. Stenqvist, L. M. Nilsson, J. A. Nilsson, J. Haanen, W. J. van Houdt, D. S. Peeper, Tumour-reactive heterotypic CD8 T cell clusters from clinical samples. Nature 649, 467-476 (2026).
10. S. P. Cullen, S. J. Martin, Mechanisms of granule-dependent killing. Cell Death Differ 15, 251-262 (2008).
11. E. J. Wherry, M. Kurachi, Molecular and cellular insights into T cell exhaustion. Nature Review Immunology 15, 486-499 (2015).
12. B. C. Miller, D. R. Sen, R. Al Abosy, K. Bi, Y. V. Virkud, M. W. LaFleur, K. B. Yates, A. Lako, K. Felt, G. S. Naik, M. Manos, E. Gjini, J. R. Kuchroo, J. J. Ishizuka, J. L. Collier, G. K. Griffin, S. Maleri, D. E. Comstock, S. A. Weiss, F. D. Brown, A. Panda, M. D. Zimmer, R. T. Manguso, F. S. Hodi, S. J. Rodig, A. H. Sharpe, W. N. Haining, Subsets of exhausted CD8(+) T cells differentially mediate tumor control and respond to checkpoint blockade. Nat Immunol 20, 326-336 (2019).
13. J. C. Riches, J. K. Davies, F. Mcclanahan, R. Fatah, S. Iqbal, S. Agrawal, A. G. Ramsay, J. G. Gribben, T cells from CLL patients exhibit features of T-cell exhaustion but retain capacity for cytokine production. Blood 121, 1612-1621 (2013).
14. X. Wu, H. Zhang, Q. Xing, J. Cui, J. Li, Y. Li, Y. Tan, S. Wang, PD-1 (+) CD8(+) T cells are exhausted in tumours and functional in draining lymph nodes of colorectal cancer patients. Br J Cancer 111, 1391-1399 (2014).
15. D. B.T., Dissertation, Ludwig Maximilian University of Munich, Munich (2010).
16. S. He, J. Li, Y. Lyu, J. Huang, K. Pu, Near-Infrared Fluorescent Macromolecular Reporters for Real-Time Imaging and Urinalysis of Cancer Immunotherapy. Journal of the American Chemical Society 142, 7075-7082 (2020).
17. J. I. Scott, S. Gutkin, O. Green, E. J. Thompson, T. Kitamura, D. Shabat, M. Vendrell, A Functional Chemiluminescent Probe for in Vivo Imaging of Natural Killer Cell Activity Against Tumours. Angewandte Chemie, International Edition 60, 5699-5703 (2021).
18. J. Xie, F. El Rami, K. Zhou, F. Simonetta, Z. Chen, X. Zheng, M. Chen, P. B. Balakrishnan, S. Y. Dai, S. Murty, I. S. Alam, J. Baker, R. S. Negrin, S. S. Gambhir, J. Rao, Multiparameter Longitudinal Imaging of Immune Cell Activity in Chimeric Antigen Receptor T Cell and Checkpoint Blockade Therapies. ACS Central Science 8, 590-602 (2022).
19. J. I. Scott, L. Mendive-Tapia, D. Gordon, N. D. Barth, E. J. Thompson, Z. Cheng, D. Taggart, T. Kitamura, A. Bravo-Blas, E. W. Roberts, J. Juarez-Jimenez, J. Michel, B. Piet, I. J. de Vries, M. Verdoes, J. Dawson, N. O. Carragher, R. A. O. Connor, A. R. Akram, M. Frame, A. Serrels, M. Vendrell, A fluorogenic probe for granzyme B enables in-biopsy evaluation and screening of response to anticancer immunotherapies. Nature Communications 13, 2366 (2022).
20. L. Xu, N. Liu, W. Zhan, Y. Deng, Z. Chen, X. Liu, G. Gao, Q. Chen, Z. Liu, G. Liang, Granzyme B Turns Nanoparticle Fluorescence “On” for Imaging Cytotoxic T Lymphocyte Activity in Vivo. ACS Nano 16, 19328-19334 (2022).
21. N. Zhao, C. Bardine, A. L. Lourenço, Y. H. Wang, Y. Huang, S. J. Cleary, D. M. Wilson, D. Y. Oh, L. Fong, M. R. Looney, M. J. Evans, C. S. Craik, In Vivo Measurement of Granzyme Proteolysis from Activated Immune Cells with PET. ACS Central Science 7, 1638-1649 (2021).
22. L. Yuan, W. Lin, S. Zhao, W. Gao, B. Chen, L. He, S. Zhu, A unique approach to development of near-infrared fluorescent sensors for in vivo imaging. J Am Chem Soc 134, 13510-13523 (2012).
23. L. Casciola-Rosen, M. Garcia-Calvo, H. G. Bull, J. W. Becker, T. Hines, N. A. Thornberry, A. Rosen, Mouse and human granzyme B have distinct tetrapeptide specificities and abilities to recruit the bid pathway. Journal of Biological Chemistry 282, 4545-4552 (2007).
24. M. E. Ivanova, N. Lukoyanova, S. Malhotra, M. Topf, J. A. Trapani, I. Voskoboinik, H. R. Saibil, The pore conformation of lymphocyte perforin. Sci Adv 8, eabk3147 (2022).
25. Rotonda, M. Garcia-Calvo, H. G. Bull, W. M. Geissler, B. M. Mckeever, C. A. Willoughby, N. A. Thornberry, J. W. Becker, The three-dimensional structure of human granzyme B compared to caspase-3, key mediators of cell death with cleavage specificity for aspartic acid in P1. Chem Biol 8, 357-368 (2001).
26. C. P. Guimaraes, M. D. Witte, C. S. Theile, G. Bozkurt, L. Kundrat, A. E. M. Blom, H. L. Ploegh, Site-specific C-terminal and internal loop labeling of proteins using sortase-mediated reactions. Nature Protocols 8, 1787-1799 (2013).

### Example 5: Materials and Methods Related to Example 4

### Immunofluorescence Staining on FFPE and Frozen Samples

For FFPE samples, immunofluorescence staining was conducted on the Ventana Discovery Ultra Staining Module (Roche/Ventana Co., Tucson, AZ). Briefly, 4 μm tissue sections were deparaffinized using EZ Prep and subjected to heat-induced antigen retrieval for 64 minutes in Cell Conditioning 1 (pH 8.4). Following sequential blocking of nonspecific binding with Casein (12 min) and endogenous peroxidases with CM1 (8 min), a triple-staining protocol was initiated. Sections were first incubated for 60 minutes with Abcam GzmB (ab4059) at a 1:100 dilution, visualized via OmniMap anti-rabbit HRP and the DISCOVERY Rhodamine 6G kit (Ventana). Subsequently, slides were incubated with Novus Biologicals Melan-A (NBP2-46603) for 60 minutes at 1:100 and visualized via the OmniMap anti-mouse HRP detection system and Cy5. Finally, Dako CD8 (GA623) antibody was applied at 1:8 dilution, incubated for 60 minutes and detected with DISCOVERY anti-mouse HQ HRP detection system and FAM. All slides were counterstained with DAPI and mounted for assessment.

Snap-frozen tumor samples are embedded in optimal cutting temperature (OCT) compound and sectioned using a cryostat. Immunofluorescence staining was performed on the Ventana Discovery Ultra. Tissue sections (5 μm) were fixed in a graduated ethanol series (50%, 70%, and 100%) and underwent heat-induced antigen retrieval using Cell Conditioning 1 (pH 8.4) for 64 minutes. Following sequential blocking of nonspecific binding with Casein (12 min) and endogenous peroxidases with CM1 (8 min), a triple-staining protocol was initiated. Sections were first incubated for 60 minutes with Bio-Rad GzmB (MCA2010) at a 1:100 dilution, visualized via OmniMap anti-mouse HRP and the DISCOVERY Rhodamine 6G kit (Ventana). Subsequently, slides were incubated with Novus Biologicals Melan-A (NBP2-46603) for 60 minutes at 1:100 and visualized via the OmniMap anti-mouse HRP detection system and Cy5. Finally, Dako CD8 (GA623) antibody was applied at 1:8 dilution, incubated for 60 minutes and detected with DISCOVERY anti-mouse HQ HRP detection system and FAM. All slides were counterstained with DAPI and mounted for assessment.

### Quantitative Analysis Immunofluorescence Staining

Fluorescence intensity analysis was performed using Imaris 10.2. Regions of tumors were carefully outlined manually on Melan-A (Cy5) channel. Individual tumor cells and CD8+ cells were segmented using adaptive thresholding in Melan-A (Cy5) and CD8a (FITC) channels, respectively. Double positive tumor and CD8+ cells were removed from the segmented CD8+ cells to exclude the so-called kiss-of-death tumor cells. For GzmB (Rhodamine 6G) intensity measurement, the background fluorescence intensity (the mean fluorescence intensity of the healthy tissues) was first subtracted from the GzmB channel. Then, the mean GzmB fluorescence intensity was obtained for tumor cells, CD8+ cells, and regions of tumors, respectively.

### Modeling of Protein Structures

The GzmB/AGB-Cy protein complex was predicated by AlphaFold and visualized by ChimeraX1.10.

### Expression and Purification of AGB-SUMO and Sortase A

Template: a single colony of BL21 (DE3) containing the pET-SUMO-DprE1 plasmid was inoculated into 100 ml of lysogeny broth (LB) with kanamycin (50 μg/ml), followed by incubation at 37° C. and 250 rpm overnight. The overnight culture was added into 500 ml of fresh LB with kanamycin (30 μg/ml) and 1 mM isopropyl β-d-1-thiogalactopyranoside. After incubation for up to 6 hours at 37° C. and 250 rpm, bacteria were harvested and pellet was frozen at −80° C. before resuspension in 22.5 ml of lysis buffer [25 mM Hepes (pH 7.4), 300 mM KCl, and 10% glycerol]. Lysozyme (2.5 ml of 7.5 mg of lysozyme per milliliter in lysis buffer) was used to lyse the bacteria. Halt proteinase inhibitor cocktail (Thermo Fisher Scientific) was added to the bacterial lysate before two rounds of metal affinity purification using TALON metal affinity resin. The affinity-purified DprE1sm fraction was eluted using lysis buffer containing up to 500 mM imidazole. DprE1sm was then dialyzed into Hepes buffer [25 mM Hepes and 10% glycerol (pH 7.4)] and stored at −80° C. Tag cleavage was achieved by overnight incubation with SUMO protease (Thermo Fisher Scientific), followed by a third-round affinity purification to remove both His6-SUMO tag and SUMO protease.

### Cell Lines and Culture Method

Raji and U87 MG cells were purchased from ATCC. Raji-RFP, Raji-Fluc, and CD19+U87-RFP cells were generated in house using commercial lentivirus (GenTarget Inc, San Diego, CA; Cellomics Technology, Halethorpe, MD). Raji cells were cultured in RPMI1640 medium with L-glutamine (Corning, 10-040-CV) supplemented with 10% fetal bovine serum (FBS; Corning, 35-010-CV) and 1% penicillin-streptomycin solution (Gibco, 15140122). CD19+ U87-RFP cells were cultured in ATCC-formulated Eagle's Minimum Essential Medium (ATCC, 30-2003) supplemented with 10% fetal bovine serum and 1% penicillin-streptomycin solution.

### Generation of GFP CD19-28ζ CAR T Cells

Peripheral blood mononuclear cells (PBMCs) were isolated from a healthy donor's whole blood by density gradient centrifugation using lymphocyte separation medium (Corning, 25-072-CI). PBMCs were collected from the interface, washed and resuspended in PBMC dilute solution. Cells were counted and used immediately or frozen in 90% fetal bovine serum+10% DMSO.

Human T cells were isolated from PBMCs using the EasySep Human T Cell Isolation Kit (STEMCELL, 17951) according to the manufacturer's instruction. Purified T cells were cultured in high-glucose RPMI1640 medium (Gibco, A10491-01) supplemented with 10% fetal bovine serum, 1% penicillin-streptomycin solution, and activated using Dynabeads human T-Activator CD3/CD28 (Gibco, 11131D) at 1:1 (beads: cells) ratio for 48 h. Human recombinant IL-2 (PeproTech, 200-02) was maintained at 200 units/mL throughout T cell culture. After activation, total T cells were frozen in 90% fetal bovine serum and 10% DMSO.

For retroviral transduction, HEK293 producer cell lines, provided as a gift from Dr. Marco Davila (Roswell Park Comprehensive Cancer Center, Buffalo, NY), produced supernatant harboring GFP CD19-28ζ CAR viral vectors. Non-tissue culture treated 6-well plates were coated with retronectin (Takara, T100A) diluted in DPBS (15 μg/ml) overnight at 4° C., blocked with 0.5% BSA in DPBS, and washed prior to use. Activated T cells (3-5×10⁶ per well) were incubated with retroviral supernatant in T cell culture medium containing 200 units/mL of IL-2, followed by centrifugation at 2,000×g for 1 hour at room temperature. Cells were incubated at 37° C. for 24 hours, and a second transduction was performed the following day. Transduced CAR T-GFP cells were subsequently expanded in T cell medium containing 200 units/mL of IL-2 for downstream assays. Transduction efficiency was calculated by over 90% with flow cytometry quantifying the expression of GFP.

### Flow Cytometry Analysis and T Cell Sorting

To evaluate the specificity of AGB-Cy7 versus commercial GB11-RB545 (BD Horizon RB545 Mouse Anti-Human Granzyme B, clone GB11), resting and CD3/28 beads activated T cells were first stained with LIVE/DEAD Fixable Blue Dead Cell Stain Kit (Invitrogen, L23105) according to the manufacturer's instructions. After one wash in PBS, surface staining was performed using anti-CD8 (BioLegend, clone RPA-T8, BV785) and anti-CD4 (Thermo Fisher, clone RPA-T4, BUV805) for 30 minutes at 4° C. in the dark. Cells were subsequently washed, fixed, and permeabilized using the BD Cytofix/Cytoperm kit following the manufacturer's protocol. For intracellular granzyme B detection, cells were incubated with either AGB-CyP (100 nM) or anti-granzyme B RB545 for 30 minutes at 4° C. in the dark. Following staining, cells were washed three times with ice-cold PBS and immediately analyzed. Data acquisition was performed on a Cytek Aurora full-spectrum 5-laser flow cytometer. Spectral unmixing and compensation were performed using single-stained controls. Data were analyzed using FlowJo software.

To analyze GFP-expressing CD19-28ζ CAR T cells infiltrating Raji-RFP tumor spheroids, spheroids were gently dissociated by repeated pipetting to generate single-cell suspensions. To enable intracellular cytokine accumulation, GolgiPlug (Brefeldin A; BD Biosciences, BDB555029) and GolgiStop (Monensin; BD Biosciences, BDB554724) were added 12 hours prior to cell harvesting. Cells were first stained with LIVE/DEAD Fixable Blue Dead Cell Stain. Surface staining was performed using anti-CD8 (BioLegend, clone: RPA-T8, BV785), anti-CD4 (Thermo, clone: RPA-T4, BUV805), anti-PD-1 (BioLegend, clone: NAT105, BV650), anti-TIM-3 (Invitrogen, clone: F38-2E2, PE-Cy5.5), anti-LAG-3 (BioLegend, clone: 11C3C65, APC-Fire 810), and anti-CD39 (BioLegend, clone: A1, BV605) for 30 minutes at 4° C. in the dark. After washing, cells were fixed and permeabilized using BD Cytofix/Cytoperm buffer, followed by intracellular staining with anti-GzmB (BioLegend, clone: GB11, AF647), anti-perforin (BioLegend, clone: dG9, BV421), and anti-INFy (BioLegend, clone: B27, PE) for 30 minutes at 4° C. in the dark. Cells were washed three times with ice-cold PBS before acquisition. Flow cytometric analysis was performed on a Cytek Aurora full-spectrum analyzer, and data were processed using FlowJo software.

For isolation of terminally exhausted CAR T cells following two weeks of continuous spheroid exposure, cells were sorted using a Sony MA900 cell sorter. Sorting gates were defined based on viability and GFP expression to selectively enrich CAR T cells.

### Confocal Imaging of 2D Cell Culture and Tumor Spheroids

Confocal fluorescence images were acquired using an inverted laser-scanning confocal microscope equipped with 20× air, 40× oil-immersion, and 63×oil-immersion objectives. Imaging parameters (laser power, gain, and detector settings) were kept constant within each experiment to allow quantitative comparison. For 2D co-culture imaging, RFP-expressing CD19+ U87 glioblastoma cells were seeded onto glass coverslips in 6-well plates and allowed to adhere overnight. GFP-expressing CD19-28ζ CAR T cells were then added at a 1:1 effector-to-target ratio and incubated for 12 hours at 37° C. in the presence of 100 nM AGB-CyP. After incubation, cells were gently washed with PBS to remove non-adherent cells and fixed at room temperature with 2% methanol-free formaldehyde (Cell Signaling Technology, 47746P) in PBS for 15 minutes. Fixed samples were washed with PBS and mounted using ProLong™ Diamond Antifade Mountant (Thermo Fisher Scientific, P36965) before imaging. Fluorescence channels were acquired sequentially. For 3D tumor spheroids, samples were fixed with 2% methanol-free formaldehyde in PBS for 20 minutes at room temperature. After fixation, spheroids were gently transferred using pipette and mounted onto glass slides for confocal imaging. Raw image files were processed and analyzed using Fiji (ImageJ). All image processing parameters were applied identically within each experimental comparison.

### Epifluorescence Imaging of Tumor Spheroids and Dissected Patient Samples

Epifluorescence imaging was performed using an EVOS M7000 Imaging System equipped with an on-stage environmental incubator (Thermo Fisher Scientific, Waltham, MA). Images were acquired using 4×, 10×, 20×, and 40× objectives. Exposure time, LED intensity, and gain settings were held constant within each experimental set to allow quantitative comparison.

To generate lymphoma tumor spheroids, 5×10⁵ Raji-RFP cells were seeded into Nunclo Sphera U-bottom ultra-low attachment 96-well plates (Thermo Fisher Scientific, 174925). Plates were centrifuged at 1,000×g for 10 minutes to facilitate cell aggregation and incubated overnight at 37° C. to allow spheroid formation. On day 2, medium was replaced with high-glucose T cell culture medium supplemented with 200 units/mL IL-2. GFP-expressing CD19-282 CAR T cells were added at the indicated effector-to-target ratios. Brightfield and fluorescence images (GFP, RFP, and AGB-CyP detected in the Cy5.5 channel) were acquired sequentially.

For archived frozen tumor specimens, macrodissection was performed on a pre-chilled stainless-steel plate maintained over dry ice to preserve tissue integrity. Dissected fragments were washed three times with DPBS and allowed to recover at 37° C. in phenol red-free RPMI 1640 basal medium for baseline (0-hour) imaging. Following baseline acquisition, AGB-CyP (200 nM final concentration) was added directly to the culture medium. Tissue fragments were longitudinally imaged without intermediate washing for up to 7 hours under controlled temperature conditions. After the incubation period, tissues were washed three times with DPBS and cultured overnight in complete T cell culture medium. On day 2, tissues were washed again three times with DPBS and subjected to a final round of imaging to assess signal retention.

Raw image files were processed and analyzed using Fiji (ImageJ). For quantitative analysis, regions of interest (ROIs) were manually defined to encompass each tissue fragment based on brightfield and fluorescence signals. Mean fluorescence intensity within each ROI was calculated. All image processing parameters, including background correction and thresholding, were applied identically across samples within each experimental comparison.

### Statistical Analysis

All statistical analyses were performed using GraphPad Prism 10 software, and all values in the study are presented as the mean±standard deviation (SD). Unpaired t-tests were used to compare two groups. Statistical significance was defined as follows: **p<0.0022, ***p<0.001. The exact sample numbers and statistical analysis are indicated in each figure legend.

### Example 6: Assay for Functional TIL Detection in Fresh Tumor Fragments Using AGB-CyP

### Overview

The present example relates to an assay that is a non-destructive fluorescence imaging method designed to detect granzyme B (gzmB) release from functional cytotoxic T lymphocytes (CTLs) within intact tumor fragments. Fresh tumor specimens are fragmented, incubated with a fluorogenic probe (AGB-CyP, for example), and imaged longitudinally to quantify CTL cytotoxic activity. Following imaging, fragments remain viable and can proceed to TIL expansion protocols.

### Materials

Freshly resected tumor surgical resections
Phenol red-free RPMI-1640 basal medium with 1% Penicillin-Streptomycin.
Complete T cell culture medium (RPMI-1640+10% human serum+1% Penicillin-Streptomycin+6000 unit/mL IL-2).
DPBS (Dulbecco's phosphate-buffered saline)
AGB-CyP probe stock solution
24-well tissue culture plates
Autoclaved fine scalpels and forceps.
Fluorescence imaging system capable of NIR detection (Ex ~690 nm/Em ~715 nm)
37° C. incubator with 5% CO₂
Fiji (ImageJ) software for image analysis

### Step 1. Tumor Specimen Collection

Fresh tumor specimens are obtained immediately following surgical resection and transported to the laboratory in sterile RPMI 1640 medium. Specimens are processed as soon as possible, ideally within 1 hour of resection, to preserve immune cell viability and cytotoxic function.

### Step 2. Tumor Fragmentation

Tumor tissues are manually dissected using sterile scalpels and forceps into 0.5 cm³ fragments (cube side length ~0.8 cm, ~0.5 g each). Fragments containing obvious necrotic regions, large blood clots, or excessive adipose tissue are excluded. Each fragment is transferred into an individual well of a 24-well culture plate containing 500 μL phenol red-free RPMI 1640 basal medium containing 1% Penicillin-Streptomycin.

### Step 3. Washing

Tumor fragments are gently washed three times with DPBS to remove residual blood and debris.

### Step 4. Tissue Equilibration

Fragments are incubated at 37° C. with 5% CO₂ for 30 minutes to allow cell recovery prior to probe exposure.

### Step 5. Baseline Imaging

Baseline brightfield and fluorescence images are acquired prior to probe addition to record background autofluorescence of the tissue fragments.

### Step 6. Probe Addition

AGB-CyP is added directly to the culture medium to achieve a final concentration of ~200 nM. Fragments are incubated at 37° C. in dark without intermediate washing.

### Step 7. Longitudinal Fluorescence Imaging

Fluorescence images are acquired at defined time intervals (typically every 1 hour) for up to 6 hours using an epifluorescence imaging system equipped with near-infrared filters (Ex ~690 nm/Em ~715 nm). All imaging parameters, including exposure time and illumination intensity, are kept constant across samples within the same experiment.

### Step 8. Probe Removal

Following the imaging period, tumor fragments are washed three times with DPBS to remove residual AGB-CyP.

### Step 9. Downstream Culture and Imaging

After washing, tumor fragments are transferred into complete T cell culture medium to culture overnight. On the second day, tumor fragments are washed three times using DPBS and imaged again.

### Step 10. Image Processing and Quantification

Raw image files can be analyzed using Fiji (ImageJ). Regions of interest (ROIs) encompassing each tumor fragment are manually defined based on brightfield and fluorescence signals. Mean fluorescence intensity within each ROI is calculated following background subtraction. All image processing parameters, including background correction, thresholding, and scaling, are applied identically across samples within each experimental comparison.

### Step 11. Tumor Selection and Downstream Processing

Tumor fragments exhibiting dynamic fluorescence signal increase over time, indicative of active granzyme B release and cytotoxic T cell activity, are prioritized for downstream processing. Selected fragments are subjected to enzymatic dissociation and ex vivo expansion of tumor-infiltrating lymphocytes (TILs) following established TIL manufacturing protocols. Fragments displaying minimal or no fluorescence activation may be processed in parallel as comparative controls or excluded from further expansion depending on experimental design.

Informal Sequence Listing

| | | SEQ |
|---|---|---|
| | | ID |
| Description | SEQUENCE (N′→C′) | NO: |
| ahGzmB | AQVQLVQSGAEVKKPGASVKVSCKASGYTFTGYYMHWVRQ | 1 |
| s17 scFV | ASGQGLEWMGLVDPEDGETIYAEKFQGRVTITADTSTDTAYM | |
| | ELSSLRSEDTAVYYCARFYRNWGQGTLVTVSSGGGGSGGG | |
| | GSGGSALSSELTQDPAVSVALGQTVRITCQGDSLRSYYASWY | |
| | QQKPGQAPVLVIYGKNNRPSGIPDRFSGSSSGNTASLTITGA | |
| | QAEDEADYYCNSRDSSGGGHVFGGGTKLTVLG | |
| 2xG4S tag | GGGGSGGGGS | 2 |
| Sort tag | LPETG | 3 |
| pelB signal | MKYLLPTAAAGLLLLAAQPAMA | 4 |
| peptide | | |
| S3FLAG tag | DYKDHDGDYKDHDIDYKDDDDK | 5 |
| aGzmB | MKYLLPTAAAGLLLLAAQPAMADYKDHDGDYKDHDIDYKDDD | 6 |
| “AGB” | DKAQVQLVQSGAEVKKPGASVKVSCKASGYTFTGYYMHWV | |
| sequence | RQASGQGLEWMGLVDPEDGETIYAEKFQGRVTITADTSTDTA | |
| | YMELSSLRSEDTAVYYCARFYRNWGQGTLVTVSSGGGGSG | |
| | GGGSGGSALSSELTQDPAVSVALGQTVRITCQGDSLRSYYAS | |
| | WYQQKPGQAPVLVIYGKNNRPSGIPDRFSGSSSGNTASLTIT | |
| | GAQAEDEADYYCNSRDSSGGGHVFGGGTKLTVLGGGGSGG | |
| | GGSLPETGGHGGGGSMSDSEVNQEAKPEVKPEVKPETHINL | |
| | KVSDGSSEIFFKIKKTTPLRRLMEAFAKRQGKEMDSLRFLYDG | |
| | IRIQADQTPEDLDMEDNDIIEAHREQIGGHHHHHH | |
| LPET | LPET | 7 |
| peptide | | |
| IEPD | IEPD | 8 |
| Peptide | | |
| Compound | Ac-IEPD-OH | 9 |
| 8 | | |
| GGGC | GGGC | 10 |
| tetrapeptide | | |
| aGzmB | GYTFTGYYMH | 11 |
| CDRH1 | | |
| aGzmB | LVDPEDGETI | 12 |
| CDRH2 | | |
| aGzmB | FYRN | 13 |
| CDRH3 | | |
| aGzmB | QGDSLRSYYAS | 14 |
| CDRL1 | | |
| aGzmB | GKNNRPS | 15 |
| CDRL2 | | |
| aGzmB | NSRDSSGGGHV | 16 |
| CDRL3 | | |
| aGzmB VH | AQVQLVQSGAEVKKPGASVKVSCKASGYTFTGYYMHWVRQ | 17 |
| Domain | ASGQGLEWMGLVDPEDGETIYAEKFQGRVTITADTSTDTAYM | |
| (CDRHs | ELSSLRSEDTAVYYCARFYRNWGQGTLVTVSSGGGGSGGG | |
| underlined, | GSGGS | |
| in order of | | |
| 1, 2, 3) | | |
| aGzmB VL | ALSSELTQDPAVSVALGQTVRITCQGDSLRSYYASWYQQKPG | 18 |
| Domain | QAPVLVIYGKNNRPSGIPDRFSGSSSGNTASLTITGAQAEDEA | |
| (CDRLs | DYYCNSRDSSGGGHVFGGGTKLTVLG | |
| underlined, | | |
| in order of | | |
| 1, 2, 3) | | |

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1. A Granzyme B (GzmB)-targeting imaging probe according to Formula 1 below (N terminus to C terminus):
X-G₃-P Formula 1
wherein X comprises an anti-GzmB (aGzmB) antibody fragment;
wherein G is glycine (G); and
wherein P is a fluorochrome, caged fluorochrome, or radioisotope.

2. The imaging probe of claim 1, wherein the aGzmB antibody fragment is an scFV that comprises:
a CDRH1 polypeptide having at least 95% sequence identity to SEQ ID NO:11;
a CDRH2 polypeptide having at least 95% sequence identity to SEQ ID NO: 12;
a CDRH3 polypeptide having at least 95% sequence identity to SEQ ID NO:13;
a CDRL1 polypeptide having at least 95% sequence identity to SEQ ID NO: 14;
a CDRL2 polypeptide having at least 95% sequence identity to SEQ ID NO: 15; and
a CDRL3 polypeptide having at least 95% sequence identity to SEQ ID NO: 16.

3. The imaging probe of claim 1, wherein the aGzmB antibody fragment is an scFV that comprises:
a VH polypeptide having at least 90% sequence identity to SEQ ID NO: 17; and
a VL polypeptide having at least 90% sequence identity to SEQ ID NO: 18.

4. The imaging probe of claim 1, wherein the aGzmB antibody fragment comprises a polypeptide having at least 85% sequence identity to anti-human GzmB (ahGzmB) scFv S17 (SEQ ID NO:1).

5. The imaging probe of claim 1, wherein the aGzmB antibody fragment further comprises a 2×G4S tag (GGGGSGGGGS, SEQ ID NO:2), a Sort tag (LPET, SEQ ID NO: 7), or both, conjugated to the C-terminus of ahGzmB scFv S17 (SEQ ID NO:1).

6. The imaging probe of claim 1, wherein the aGzmB antibody fragment further comprises a pelB signal peptide (MKYLLPTAAAGLLLLAAQPAMA, SEQ ID NO:4), a S3FLAG tag (DYKDHDGDYKDHDIDYKDDDDK, SEQ ID NO: 5), or both, conjugated to the N-terminus of ahGzmB scFv S17 (SEQ ID NO:1).

7. The imaging probe of claim 1, wherein the anti-GzmB (aGzmB) antibody fragment comprises SEQ ID NO:6.

8. The imaging probe of claim 1, wherein the fluorochrome is a near-infrared (NIR) cyanine dye, a hemicyanine dye, a coumarin-based dye, a fluorescein-based dye, a rhodamine-based dye, a BODIPY-Based Dye, a BODIPY-Based NIR Dye, a Xanthene-Based dye, a Xanthene-Based NIR Dye, a cyanine-based dyes, a porphyrin dye, a phthalocyanine dye, a squaraine dye.

9. The imaging probe of claim 1, wherein the fluorochrome comprises Cy5.5, Cy7, IRDye800CW, a Silicon Rhodamine, a Janelia Fluor 646, a Janelia Fluor 669, a Janelia Fluor 774, Squaraine Dyes, ICG, or IR-800.

10. The imaging probe of claim 1, wherein the caged fluorochrome is a NIR caged cyanine or hemicyanine dye.

11. The imaging probe of claim 1, wherein the caged fluorochrome comprises CyP.

12. The imaging probe of claim 1, wherein the radioisotope comprises Zirconium-89 (⁸⁹Zr), ⁸⁹Zr chelated with desferrioxamine (DFO) or GGG-DFO, Copper-64 (⁶⁴Cu) and Fluorine-18 (¹⁸F), Technetium-99m, Iodine-131, Gallium-67, or Radium-223.

13. The imaging probe of claim 1, wherein “G₃-P” has the following structure (N terminus to C terminus):

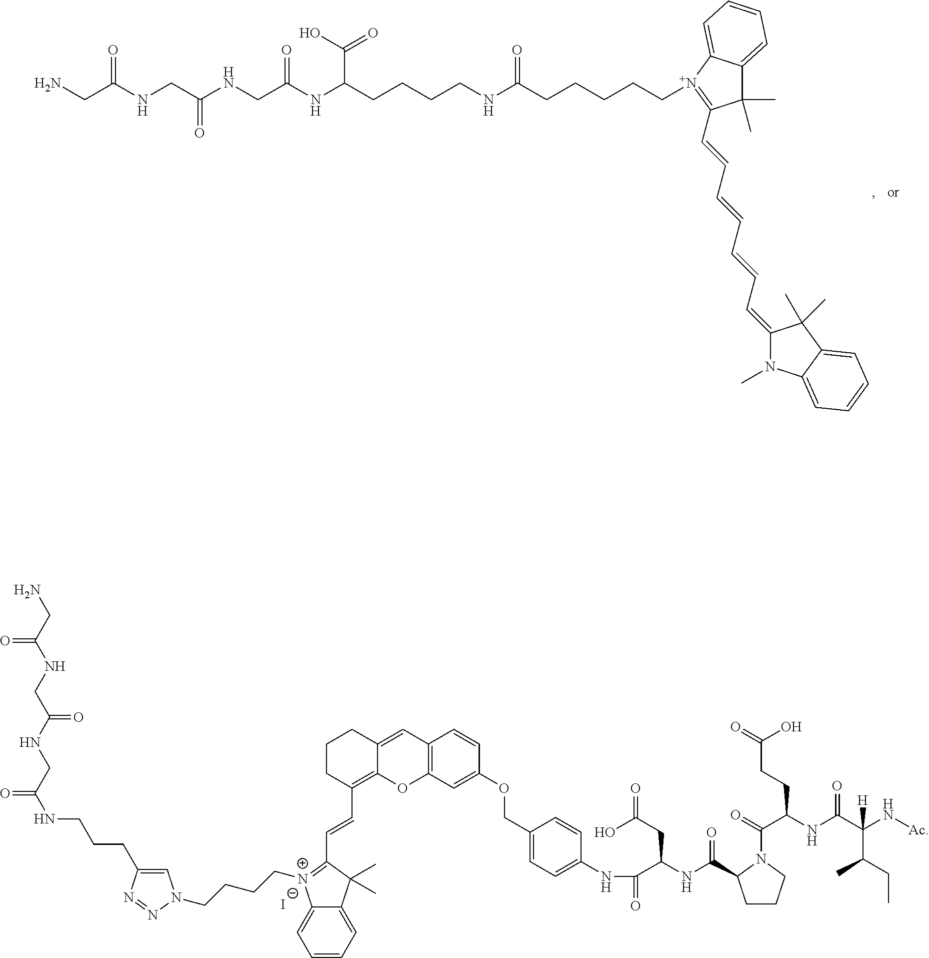 [SMILES: CN1/C(=C/C=C/C=C/C=C/C2=[N+](CCCCCC(=O)NCCCCC(NC(=O)CNC(=O)CNC(=O)CN)C(=O)O)c3ccccc3C2(C)C)C(C)(C)c2ccccc21 ; CC[C@@H](C)[C@@H](NC(C)=O)C(=O)N[C@H](CCC(=O)O)C(=O)N1CCC[C@H]1C(=O)N[C@H](CC(=O)O)C(=O)Nc1ccc(COc2ccc3c(c2)OC2=C(/C=C/C4=[N+](CCCCn5cc(CCCNC(=O)CNC(=O)CNC(=O)CN)nn5)c5ccccc5C4(C)C)CCCC2=C3)cc1 ; [I-]]

14. A method of using a Granzyme B (GzmB)-targeting imaging probe, comprising:
contacting a sample with an imaging probe of claim 1;
incubating the contacted sample at a temperature for a period of time; and
imaging the incubated sample to determine a probe level.

15. The method of claim 14, wherein the sample is a tumor resection from a tumor of a subject or a tumor or suspected tumor in a subject.

16. The method of claim 15, wherein the tumor is a metastatic melanoma, a sarcoma, a pancreatic tumor, an ovarian tumor, a colorectal tumor, a non-small cell lung cancer (NSCLC), and a triple-negative breast cancer tumor.

17. The method of claim 15, wherein the subject is undergoing or suspected to undergo tumor-infiltrating lymphocyte (TIL) therapy or has a metastatic or unresectable melanoma that has progressed on standard immune checkpoint inhibitors and BRAF-targeted therapies, a sarcoma, a pancreatic cancer, an ovarian cancer, a colorectal cancer, a non-small cell lung cancer, or a triple-negative breast cancer.

18. The method of claim 14, further comprising identifying samples for use in future TIL therapy for the subject based on a determined probe level above a threshold.

19. The method of claim 14 wherein the determined probe level is a measured fluorescence intensity.

20. A kit, comprising:
the imaging probe of claim 1; and
Instructions for use.


SEQUENCE LISTING (18 sequences)

SEQ ID NO: 1 - protein, 238 aa, synthetic construct
AQVQLVQSGAEVKKPGASVKVSCKASGYTFTGYYMHWVRQASGQGLEWMGLVDPEDGETIYAEKFQGRVTITADTSTDTAYMELSSLRSEDTAVYYCARFYRNWGQGTLVTVSSGGGGSGGGGSGGSALSSELTQDPAVSVALGQTVRITCQGDSLRSYYASWYQQKPGQAPVLVIYGKNNRPSGIPDRFSGSSSGNTASLTITGAQAEDEADYYCNSRDSSGGGHVFGGGTKLTVLG

SEQ ID NO: 2 - protein, 10 aa, synthetic construct
GGGGSGGGGS

SEQ ID NO: 3 - protein, 5 aa, synthetic construct
LPETG

SEQ ID NO: 4 - protein, 22 aa, synthetic construct
MKYLLPTAAAGLLLLAAQPAMA

SEQ ID NO: 5 - protein, 22 aa, synthetic construct
DYKDHDGDYKDHDIDYKDDDDK

SEQ ID NO: 6 - protein, 407 aa, synthetic construct
MKYLLPTAAAGLLLLAAQPAMADYKDHDGDYKDHDIDYKDDDDKAQVQLVQSGAEVKKPGASVKVSCKASGYTFTGYYMHWVRQASGQGLEWMGLVDPEDGETIYAEKFQGRVTITADTSTDTAYMELSSLRSEDTAVYYCARFYRNWGQGTLVTVSSGGGGSGGGGSGGSALSSELTQDPAVSVALGQTVRITCQGDSLRSYYASWYQQKPGQAPVLVIYGKNNRPSGIPDRFSGSSSGNTASLTITGAQAEDEADYYCNSRDSSGGGHVFGGGTKLTVLGGGGSGGGGSLPETGGHGGGGSMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGHHHHHH

SEQ ID NO: 7 - protein, 4 aa, synthetic construct
LPET

SEQ ID NO: 8 - protein, 4 aa, synthetic construct
IEPD

SEQ ID NO: 9 - protein, 4 aa, synthetic construct
IEPD

SEQ ID NO: 10 - protein, 4 aa, synthetic construct
GGGC

SEQ ID NO: 11 - protein, 10 aa, synthetic construct
GYTFTGYYMH

SEQ ID NO: 12 - protein, 10 aa, synthetic construct
LVDPEDGETI

SEQ ID NO: 13 - protein, 4 aa, synthetic construct
FYRN

SEQ ID NO: 14 - protein, 11 aa, synthetic construct
QGDSLRSYYAS

SEQ ID NO: 15 - protein, 7 aa, synthetic construct
GKNNRPS

SEQ ID NO: 16 - protein, 11 aa, synthetic construct
NSRDSSGGGHV

SEQ ID NO: 17 - protein, 127 aa, synthetic construct
AQVQLVQSGAEVKKPGASVKVSCKASGYTFTGYYMHWVRQASGQGLEWMGLVDPEDGETIYAEKFQGRVTITADTSTDTAYMELSSLRSEDTAVYYCARFYRNWGQGTLVTVSSGGGGSGGGGSGGS

SEQ ID NO: 18 - protein, 111 aa, synthetic construct
ALSSELTQDPAVSVALGQTVRITCQGDSLRSYYASWYQQKPGQAPVLVIYGKNNRPSGIPDRFSGSSSGNTASLTITGAQAEDEADYYCNSRDSSGGGHVFGGGTKLTVLG